(19) United States
(12) Patent Application Publication
(10) Pub. No.: US 2024/0246969 A1
(43) Pub. Date: Jul. 25, 2024

(54) HETEROCYCLIC DERIVATIVES AS CAMKK2 INHIBITORS

(71) Applicant: Bristol-Myers Squibb Company, Princeton, NJ (US)

(72) Inventors: Young Chen, San Diego, CA (US); Michael Wallace, San Diego, CA (US); Zhe Nie, San Diego, CA (US); Laura D'Agostino, Sudbury, MA (US)

(21) Appl. No.: 18/560,090

(22) PCT Filed: May 10, 2022
(86) PCT No.: PCT/US2022/028512

§ 371 Date: Nov. 9, 2023

Related U.S. Application Data
(60) Provisional application No. 63/187,048, filed on May 11, 2021.

(51) Int. Cl. C07D 471/04; A61K 31/437; A61K 31/47; A61K 31/4985; A61K 31/5025; A61K 31/517; A61K 31/519; C07D 215/233; C07D 239/80; C07D 487/04

(52) U.S. Cl. CPC C07D 471/04; A61K 31/437; A61K 31/47; A61K 31/4985; A61K 31/5025; A61K 31/517; A61K 31/519; C07D 215/233; C07D 239/80; C07D 487/04

(57) ABSTRACT

The present invention relates generally to compositions and methods for treating cancer, e.g. by using the compounds of formula (I). Provided herein are substituted bicyclic heteroaryl derivative compounds and pharmaceutical compositions comprising said compounds. The subject compounds and compositions are useful for inhibition of Calcium/calmodulin-dependent protein kinase kinase 2. Furthermore, the subject compounds and compositions are useful for the treatment of cancer.

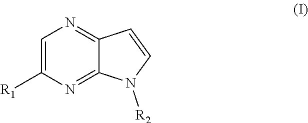

### TECHNICAL FILED

The present invention relates to compounds and methods for the treatment of cancer.

### BACKGROUND OF THE INVENTION

Tumor-associated myeloid cells play a pivotal role in the regulation of processes that control tumor growth and metastasis, and their accumulation in cancer tumors has been identified as an important negative prognostic factor. Prior art has shown that depletion of Calcium/calmodulin-dependent protein kinase kinase 2 (CaMKK2) activity is associated with the accumulation of macrophages expressing high levels of the major histocompatibility molecule class II molecule I-A (MHC II I-A), and CD8+ T-cells within the tumor microenvironment. See WO 2018/027223. Treatment with CaMKK2 inhibitors is also shown to block tumor growth and facilitate reprogramming of the microenvironment. See WO 2018/027223. In human breast cancer biopsies, CaMKK2 expression levels correlate with tumor grade, and in high-grade tumors, both tumor cells and tumor-associated macrophages express high levels of this enzyme. See WO 2018/027223. These findings implicate CaMKK2 as a macrophage specific checkpoint, and demonstrate that CaMKK2 inhibition would be an innovative therapeutic strategy for treating cancer through reprogramming the tumor microenvironment.

In addition to cancer treatment, CaMKK2 inhibition is shown to therapeutically promotes weight loss, which would have significant medical and social benefits. CaMKK2-null mice show a sustained reduction in feeding on a high-fat diet and have resultingly lower body weights, reduced adiposity, and improved glucose sensitivity relative to their WT littermates (Anderson, K. A. et al. Cell Metab. 2008, 7, 377). From a mechanistic standpoint, CaMKK2-null mice are acutely resistant to ghrelin-induced food intake, and eat less than their wile type (WT) counterparts upon refeeding after fasting, similar to NPY-depleted mice. These latter observations are corroborated by pharmacological inhibition of CaMKK2 using STO-609 delivered via i.c.v. administration (Anderson, K. A. et al. Cell Metab. 2008, 7, 377), or using 2,4-diaryl 7-azaindoles 4t delivered via oral administration (Price, D J et al. Bioorg. Med. Chem. Lett. 2018 Jun. 1; 28(10): 1958-1963).

Therefore, CaMKK2 is an attractive therapeutic target for the treatment of cancer and obesity. Several small molecule inhibitors of CaMKK2 activity are available, such as STO-609 and N28464-13-Al (referred to as GSKi), or derivatives thereof. STO-609 is disclosed, for example, in US Patent Publication No. 2013/0253035. STO-609 is a selective, cell-permeable inhibitor of CaMKK proteins. STO-609 has an approximately 5-fold higher affinity for CaMKK2 than CaMKK1 and is often used in vivo or in vitro to suppress the CaMKK2-AMPK pathway. STO-609 may be obtained from commercial suppliers (e.g., Torcis Biosciences). Other selective and targeted inhibitors of CaMKK2, such as GSKi, are in development and known to those of skill in the art. However, highly potent and selective CaMKK2 inhibitors are still in need for the development of effective and safe therapeutics.

### SUMMARY OF THE INVENTION

This invention provides potent and selective CaMKK2 inhibitors that are useful for cancer treatment. Provided herein are substituted bicyclic heteroaryl derivative compounds and pharmaceutical compositions comprising said compounds. The subject compounds and compositions are useful for inhibition of CaMKK2. Furthermore, the subject compounds and compositions are useful for the treatment of cancer, such as acute myeloid leukemia (AML), acute lymphoblastic leukemia (ALL), small cell lung cancer (SCLC), non-small cell lung cancer (NSCLC), neuroblastoma, small round blue cell tumors, glioblastoma, prostate cancer, breast cancer, bladder cancer, lung cancer and/or melanoma and the like. The substituted bicyclic heteroaryl derivative compounds described herein are based upon a central pyrrolopyrazine, pyrazolopyrimidine, pyrazolopyridine, pyrrolopyridazine, quinazoline or quinoline ring system, or the like. Said pyrrolopyrazine, pyrazolopyrimidine, pyrazolopyridine, pyrrolopyridazine, quinazoline or quinoline ring systems are further substituted with an aryl group and additional groups, such as halogen, carbonyl, aryl, alkyl, alkoxy, cycloalkyl, heteroaryl, or amine groups.

One embodiment provides a compound having the structure of Formula (I), or a pharmaceutically acceptable salt thereof,

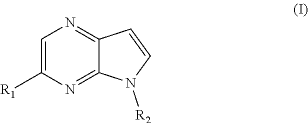

wherein,
R₁ is alkyl, cycloalkyl, optionally substituted aryl, optionally substituted heteroaryl, or amine; and
R₂ is optionally substituted aryl.

Another embodiment provides a compound of Formula (I), wherein R₁ is C3 cycloalkyl.

Another embodiment provides a compound of Formula (I), wherein R₁ is aryl optionally substituted by halogen, alkyl, or cycloalkyl. Another embodiment provides a compound of Formula (I), wherein R₁ is aryl optionally substituted by methyl. Another embodiment provides a compound of Formula (I), wherein R₁ is aryl optionally substituted by C3 cycloalkyl.

Another embodiment provides a compound of Formula (I), wherein R₁ is heteroaryl optionally substituted by halogen or alkyl. Another embodiment provides a compound of Formula (I), wherein R₁ is heteroaryl optionally substituted by methyl. Another embodiment provides a compound of Formula (I), wherein R₁ is bicyclic heteroaryl.

Another embodiment provides a compound of Formula (I), wherein R₂ is aryl optionally substituted by at least one substituent chosen from cycloalkyl, carbonyl, amine, —CN, heterocyclyl, heterocyclyloxy, heterocyclylalkyl or heteroaryl. Another embodiment provides a compound of Formula (I), wherein R₂ is aryl optionally substituted by cycloalkyl, wherein the cycloalkyl is C5-cycloalkyl. Another embodiment provides a compound of Formula (I), wherein R₂ is aryl optionally substituted by carbonyl, wherein the carbonyl is chosen from —C(═O)OH, —C(═O)NH—CN, or —C(═O)NH—SO₂—CF₃. Another embodiment provides a compound of Formula (I), wherein R₂ is aryl optionally substituted by amine, wherein the amine is chosen from —NH-alkyl, —NH-cycloalkyl, —NH— cycloalkylalkyl, or —NH-heterocyclyl. Another embodiment provides a compound of Formula (I), wherein R₂ is aryl optionally substituted by heterocyclyl, wherein the heterocyclyl is 5-6 membered heterocyclyl containing N. Another embodiment provides a compound of Formula (I), wherein R₂ is aryl optionally substituted by heteroaryl, wherein the heteroaryl is tetrazole.

One embodiment provides a compound having the structure of Formula (IIa), or a pharmaceutically acceptable salt thereof,

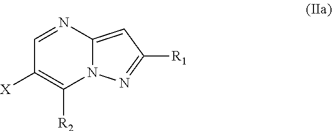

wherein,
R₁ is alkoxy, cycloalkyl, optionally substituted aryl, optionally substituted heteroaryl, or amine;
R₂ is optionally substituted aryl; and
X is hydrogen or halogen.

Another embodiment provides a compound of Formula (IIa), wherein R₁ is alkoxy chosen from methoxy or ethoxy. Another embodiment provides a compound of Formula (IIa), wherein R₁ is C3 cycloalkyl. Another embodiment provides a compound of Formula (IIa), wherein R₁ is aryl optionally substituted by halogen and alkoxy. Another embodiment provides a compound of Formula (IIa), wherein R₁ is aryl optionally substituted by methoxy. Another embodiment provides a compound of Formula (IIa), wherein R₁ is aryl optionally substituted by —O-cycloalkylalkyl. Another embodiment provides a compound of Formula (IIa), wherein R₁ is heteroaryl optionally substituted by methyl. Another embodiment provides a compound of Formula (IIa), wherein R₁ is pyrazole substituted by methyl.

Another embodiment provides a compound of Formula (IIa), wherein R₂ is aryl optionally substituted by at least one substituent chosen from carbonyl, alkoxy, or cycloalkyl. Another embodiment provides a compound of Formula (IIa), wherein R₂ is aryl optionally substituted by carbonyl, wherein the carbonyl is —C(═O)OH or —C(═O)— amine. Another embodiment provides a compound of Formula (IIa), wherein R₂ is aryl optionally substituted by alkoxy, wherein the alkoxy is methoxy. Another embodiment provides a compound of Formula (IIa), wherein R₂ is aryl optionally substituted by cycloalkyl, wherein the cycloalkyl is C5 cycloalkyl.

One embodiment provides a compound having the structure of Formula (IIb), or a pharmaceutically acceptable salt thereof,

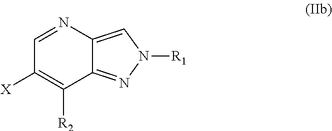

wherein,
R₁ is optionally substituted aryl;
R₂ is optionally substituted aryl; and
X is hydrogen or halogen.

Another embodiment provides a compound of Formula (IIb), wherein R₁ is aryl optionally substituted by at least one substituent chosen from halogen, alkyl, CF₃, alkoxy, —O—CF₃, or cycloalkyl. Another embodiment provides a compound of Formula (IIb), wherein R₁ is aryl optionally substituted by methyl. Another embodiment provides a compound of Formula (IIb), wherein R₁ is aryl optionally substituted by methoxy. Another embodiment provides a compound of Formula (IIb), wherein R₁ is aryl optionally substituted by C3-cycloalkyl.

Another embodiment provides a compound of Formula (IIb), wherein R₂ is aryl optionally substituted by at least one substituent chosen from carbonyl, alkoxy, amine, or cycloalkyl. Another embodiment provides a compound of Formula (IIb), wherein R₂ is aryl optionally substituted by carbonyl, wherein the carbonyl is chosen from —C(═O)OH or —C(═O)-amine. Another embodiment provides a compound of Formula (IIb), wherein R₂ is aryl optionally substituted by alkoxy, wherein the alkoxy is methoxy. Another embodiment provides a compound of Formula (IIb), wherein R₂ is aryl optionally substituted by amine, wherein the amine is —NH-cycloalkyl. Another embodiment provides a compound of Formula (IIb), wherein R₂ is aryl optionally substituted by cycloalkyl, wherein the cycloalkyl is C5 cycloalkyl.

One embodiment provides a compound having the structure of Formula (IIc), or a pharmaceutically acceptable salt thereof,

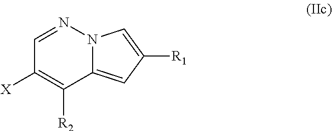

wherein,
R₁ is optionally substituted aryl;
R₂ is optionally substituted aryl; and
X is hydrogen or halogen.

Another embodiment provides a compound of Formula (IIc), wherein R₁ is aryl optionally substituted by at least one substituent chosen from halogen or alkyl. Another embodiment provides a compound of Formula (IIc), wherein R₁ is aryl optionally substituted by alkyl, wherein the alkyl is methyl.

Another embodiment provides a compound of Formula (IIc), wherein R₂ is aryl optionally substituted by at least one substituent chosen from carbonyl or cycloalkyl. Another embodiment provides a compound of Formula (IIc), wherein R₂ is aryl optionally substituted by carbonyl, wherein the carbonyl is —C(═O)OH. Another embodiment provides a compound of Formula (IIc), wherein R₂ is aryl optionally substituted by cycloalkyl, wherein the cycloalkyl is C5 cycloalkyl.

One embodiment provides a compound having the structure of Formula (III), or a pharmaceutically acceptable salt thereof,

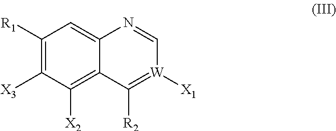

wherein
W is carbon or nitrogen;
R₁ is alkoxy, cyclylalkoxy, heterocyclyl, or heteroaryl;
R₂ is optionally substituted aryl; and
X₁, X₂, and X₃ is independently hydrogen or halogen.

Another embodiment provides a compound of Formula (III), wherein R₁ is ethoxy. Another embodiment provides a compound of Formula (III), wherein R₁ is C3 cyclylalkoxy. Another embodiment provides a compound of Formula (III), wherein R₁ is C5 heterocyclyl containing at least one nitrogen. Another embodiment provides a compound of Formula (III), wherein R₁ is C5 heteroaryl containing at least one nitrogen.

Another embodiment provides a compound of Formula (III), wherein R₂ is aryl optionally substituted by at least one substituent chosen from halogen, carbonyl, cycloalkyl, or heterocyclyl. Another embodiment provides a compound of Formula (III), wherein R₂ is aryl optionally substituted by carbonyl, wherein the carbonyl is —C(═O)OH. Another embodiment provides a compound of Formula (III), wherein R₂ is aryl optionally substituted by cycloalkyl, wherein the cycloalkyl is C3-C5 cycloalkyl. Another embodiment provides a compound of Formula (III), wherein R₂ is aryl optionally substituted by heterocyclyl, wherein the heterocyclyl is C3-C6 heterocyclyl containing at least one nitrogen.

One embodiment provides a pharmaceutical composition comprising a compound of Formula (I), or a pharmaceutically acceptable salt thereof, and a pharmaceutically acceptable excipient. One embodiment provides a pharmaceutical composition comprising a compound of Formula (IIa), or a pharmaceutically acceptable salt thereof, and a pharmaceutically acceptable excipient. One embodiment provides a pharmaceutical composition comprising a compound of Formula (IIb), or a pharmaceutically acceptable salt thereof, and a pharmaceutically acceptable excipient. One embodiment provides a pharmaceutical composition comprising a compound of Formula (IIc), or a pharmaceutically acceptable salt thereof, and a pharmaceutically acceptable excipient. One embodiment provides a pharmaceutical composition comprising a compound of Formula (III), or a pharmaceutically acceptable salt thereof, and a pharmaceutically acceptable excipient.

One embodiment provides a method of regulating gene transcription in a cell comprising inhibiting CaMKK2 activity by exposing the CaMKK2 enzyme to a compound of Formula (I). One embodiment provides a method of regulating gene transcription in a cell comprising inhibiting CaMKK2 activity by exposing the CaMKK2 enzyme to a compound of Formula (IIa). One embodiment provides a method of regulating gene transcription in a cell comprising inhibiting CaMKK2 activity by exposing the CaMKK2 enzyme to a compound of Formula (IIb). One embodiment provides a method of regulating gene transcription in a cell comprising inhibiting CaMKK2 activity by exposing the CaMKK2 enzyme to a compound of Formula (IIc). One embodiment provides a method of regulating gene transcription in a cell comprising inhibiting CaMKK2 activity by exposing the CaMKK2 enzyme to a compound of Formula (III).

One embodiment provides a method of treating cancer in a patient in need thereof, comprising administering to the patient a therapeutically effective amount of a compound of Formula (I), or a pharmaceutically acceptable salt thereof. One embodiment provides a method of treating cancer in a patient in need thereof, comprising administering to the patient a therapeutically effective amount of a compound of Formula (IIa), or a pharmaceutically acceptable salt thereof. One embodiment provides a method of treating cancer in a patient in need thereof, comprising administering to the patient a therapeutically effective amount of a compound of Formula (IIb), or a pharmaceutically acceptable salt thereof. One embodiment provides a method of treating cancer in a patient in need thereof, comprising administering to the patient a therapeutically effective amount of a compound of Formula (IIc), or a pharmaceutically acceptable salt thereof. One embodiment provides a method of treating cancer in a patient in need thereof, comprising administering to the patient a therapeutically effective amount of a compound of Formula (III), or a pharmaceutically acceptable salt thereof

### DETAILED DESCRIPTION OF THE INVENTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

All patents and other publications identified are incorporated herein by reference for the purpose of describing and disclosing, for example, the methodologies described in such publications that might be used in connection with the present invention, but are not to provide definitions of terms inconsistent with those presented herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on information available to the applicants and do not constitute any admission as to the correctness of the dates or contents of these documents.

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either.” Thus, unless context indicates otherwise, the word “or” means any one member of a particular list and also includes any combination of members of that list. Other than in the operating examples, or where otherwise indicated, all numbers expressing quantities of ingredients or reaction conditions used herein should be understood as modified in all instances by the term “about.”

### Definitions

As used in the specification and appended claims, unless specified to the contrary, the following terms have the meaning indicated below.

“Amino” refers to the —NH₂ radical.

“Cyano” refers to the CN radical.

“Nitro” refers to the NO2 radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH2 radical.

“Alkyl” refers to a straight or branched hydrocarbon chain radical consisting solely of carbon and hydrogen atoms, containing no unsaturation, having from one to fifteen carbon atoms (e.g., C1-C15 alkyl). In certain embodiments, an alkyl comprises one to thirteen carbon atoms (e.g., C1-C13 alkyl). In certain embodiments, an alkyl comprises one to eight carbon atoms (e.g., C1-C8 alkyl). In other embodiments, an alkyl comprises one to five carbon atoms (e.g., C1-C5 alkyl). In other embodiments, an alkyl comprises one to four carbon atoms (e.g., C1-C4 alkyl). In other embodiments, an alkyl comprises one to three carbon atoms (e.g., C1-C3 alkyl). In other embodiments, an alkyl comprises one to two carbon atoms (e.g., C1-C2 alkyl). In other embodiments, an alkyl comprises one carbon atom (e.g., C1 alkyl). In other embodiments, an alkyl comprises five to fifteen carbon atoms (e.g., C5-C15 alkyl). In other embodiments, an alkyl comprises five to eight carbon atoms (e.g., C5-C8 alkyl). In other embodiments, an alkyl comprises two to five carbon atoms (e.g., C2-C5 alkyl). In other embodiments, an alkyl comprises three to five carbon atoms (e.g., C3-C5 alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo,
trimethylsilanyl, —OR^{a}, —SR^{a}, —OC(O)—R^{a}, —N(R^{a})₂, —C(O)R^{a},
—C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)R^{a} (where t is 1 or 2), —S(O)ₜOR^{a} (where t is 1 or 2), —S(O)ₜR^{a} (where t is 1 or 2)
and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop 1 enyl (i.e., allyl), but 1 enyl, pent 1 enyl, penta 1,4 dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂,
—N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR, —OC(O)—R^{a}, —N(R^{a})₂,
—C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. In certain embodiments, an alkylene comprises one to eight carbon atoms (e.g., C1-C8 alkylene). In other embodiments, an alkylene comprises one to five carbon atoms (e.g., C1-C5 alkylene). In other embodiments, an alkylene comprises one to four carbon atoms (e.g., C1-C4 alkylene). In other embodiments, an alkylene comprises one to three carbon atoms (e.g., C1-C3 alkylene). In other embodiments, an alkylene comprises one to two carbon atoms (e.g., C1-C2 alkylene). In other embodiments, an alkylene comprises one carbon atom (e.g., C1 alkylene). In other embodiments, an alkylene comprises five to eight carbon atoms (e.g., C5-C8 alkylene). In other embodiments, an alkylene comprises two to five carbon atoms (e.g., C2-C5 alkylene). In other embodiments, an alkylene comprises three to five carbon atoms (e.g., C3-C5 alkylene). Unless stated otherwise specifically in the specification, an alkylene chain is optionally substituted by one or more of the following substituents: halo, cyano, nitro, oxo, thioxo, imino, oximo,
trimethylsilanyl, —OR^{a}, —SR, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted
heteroarylalkyl, —R^{b}—OR^{a}, —R^{b}—OC(O)—R^{a}, —R^{b}—OC(O)—OR^{a}, —R^{b}—OC(O)—N(R^{a})₂,
—R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula R^{c} aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —Rd aryl where Rd is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula Re aryl, where Re is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O R^{c} aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl may be saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7 dimethyl bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —OR^{a}, —SR^{a},
—OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each Rb is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c} carbocyclyl where R^{c} is an alkylene chain as defined above. The alkylene chain and the carbocyclyl radical is optionally substituted as defined above.

“Carbocyclylalkoxy” refers to a radical bonded through an oxygen atom of the formula —O—R^{c} carbocyclyl where R^{c} is an alkylene chain as defined above. The alkylene chain and the carbocyclyl radical is optionally substituted as defined above.

“Carbocyclylalkynyl” refers to a radical of the formula R^{c} carbocyclyl, where R^{c} is an alkynylene chain as defined above. The carbocyclyl part of the carbocyclylalkynyl radical is optionally substituted as described above for an carbocyclyl group. In some embodiments the carbocyclyl group is a cycloalkyl group. The alkynylene chain part of the carbocyclylalkynyl radical is optionally substituted as defined above for an alkynylene chain.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

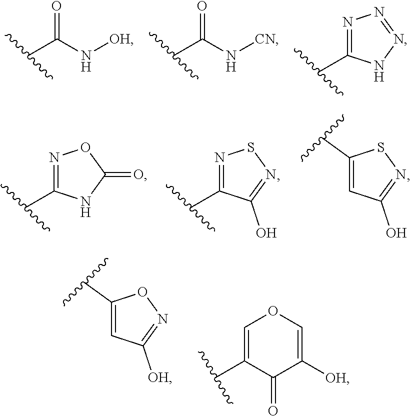

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2 trifluoroethyl, 1 fluoromethyl 2 fluoroethyl, and the like. The alkyl part of the fluoroalkyl radical may be optionally substituted as defined above for an alkyl group.

“Heterocyclyl” refers to a stable 3 to 18 membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1 oxo thiomorpholinyl, and 1,1 dioxo thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted

heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each Rb is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c} heterocyclyl where R^{c} is an alkylene chain as defined above. If the heterocyclyl is a nitrogen containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c} heterocyclyl where R^{c} is an alkylene chain as defined above. If the heterocyclyl is a nitrogen containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3 to 18 membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted
heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—OR^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c} heteroaryl, where R^{c} is an alkylene chain as defined above. If the heteroaryl is a nitrogen containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c} heteroaryl, where R^{c} is an alkylene chain as defined above. If the heteroaryl is a nitrogen containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein may contain one or more asymmetric centers and may thus give rise to enantiomers, diastereomers, and other stereoisomeric forms that may be defined, in terms of absolute stereochemistry, as (R) or (S). Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein may, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

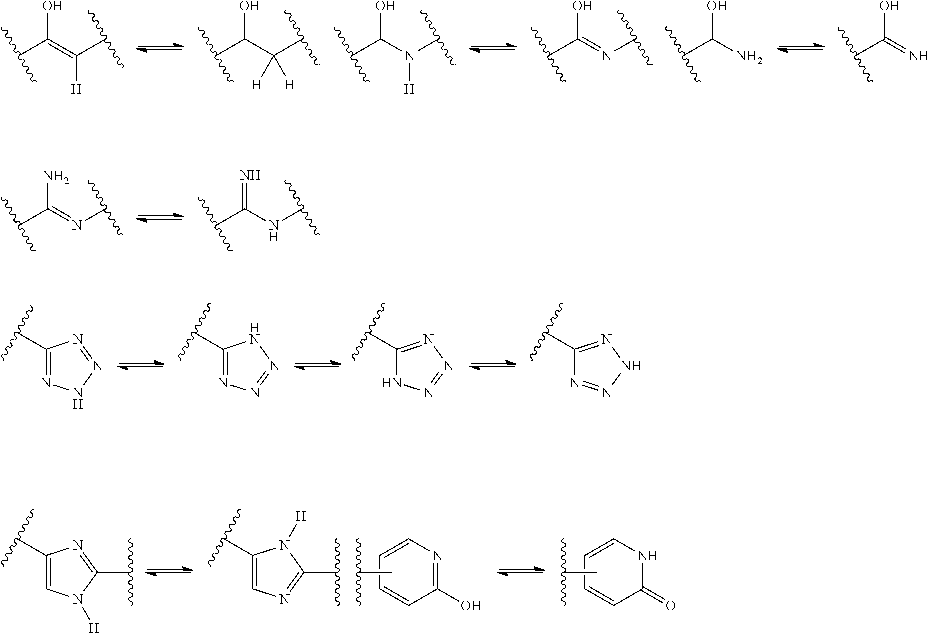

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts. A pharmaceutically acceptable salt of any one of the substituted heterocyclic derivative compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2 dimethylaminoethanol, 2 diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N ethylpiperidine, polyamine resins and the like. See Berge et al., supra.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7 9, 21 24 (Elsevier, Amsterdam).

A discussion of prodrugs is provided in Higuchi, T., et al., “Prodrugs as Novel Delivery Systems,” A.C.S. Symposium Series, Vol. 14, and in Bioreversible Carriers in Drug Design, ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by 13C- or 14C-enriched carbon are within the scope of the present disclosure.

Pharmaceutical compositions including any of the compositions described herein are also provided. The pharmaceutical compositions may include a pharmaceutical carrier, excipient, or diluent, which are nontoxic to the cell or subject being exposed thereto at the dosages and concentrations employed. Often a pharmaceutical diluent is in an aqueous pH buffered solution. Examples of pharmaceutical carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ brand surfactant, polyethylene glycol (PEG), and PLURONICS™ surfactant.

Methods of treating cancer in a subject are also provided. The methods may include administering any of the compositions or pharmaceutical compositions described herein to a subject in an amount effective to treat the cancer. As used herein, the “subject” may be any mammal, suitably a human, or domesticated animal such as a dog, cat, horse, cow, pig, or a mouse or rat. Exemplary cancers in accordance with the present invention include, without limitation, primary and metastatic breast, ovarian, lymphoma, myeloma, pancreatic, prostate, bladder, lung, osteosarcoma, pancreatic, gastric, esophageal, colon, skin cancers (basal and squamous carcinoma; melanoma), testicular, colorectal, urothelial, renal cell, hepatocellular, leukemia, and central nervous system cancers or pre-cancers.

Treating cancer includes, without limitation, reducing the number of cancer cells or the size of a tumor in the subject, reducing progress of a cancer to a more aggressive form (i.e. maintaining the cancer in a form that is susceptible to a therapeutic agent), reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with cancer or at risk of developing cancer or facing a cancer recurrence. Treatment includes improvement in the condition of the subject (e.g., in one or more symptoms), delay in the progression of the disease, delay in the onset of symptoms or slowing the progression of symptoms, etc.

An “effective amount” or a “therapeutically effective amount” as used herein means the amount of a composition that, when administered to a subject for treating a state, disorder or condition is sufficient to effect a treatment (as defined above). The therapeutically effective amount will vary depending on the compound, formulation or composition, the disease and its severity and the age, weight, physical condition and responsiveness of the subject to be treated.

It will be appreciated that the specific dosage of a CaMKK2 inhibitor administered in any given case will be adjusted in accordance with the composition or compositions being administered, the volume of the composition that can be effectively delivered to the site of administration, the disease to be treated or inhibited, the condition of the subject, and other relevant medical factors that may modify the activity of the compositions or the response of the subject, as is well known by those skilled in the art. For example, the specific dose of a CaMKK2 inhibitor for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the compositions described herein and of a known agent, such as by means of an appropriate conventional pharmacological protocol. The compositions can be given in a single dose schedule, or in a multiple dose schedule.

The maximal dosage of a CaMKK2 inhibitor for a subject is the highest dosage that does not cause undesirable or intolerable side effects. The number of variables in regard to an individual treatment regimen is large, and a considerable range of doses is expected. The route of administration will also impact the dosage requirements. It is anticipated that dosages of the compositions will treat cancer by, for example, by reducing tumor size or decreasing the rate of tumor growth by least 10%, 20%, 30%, 40%, 50%, 60%, 70%, 80%, 90%, 100% or more as compared to no treatment.

The effective dosage amounts of a CaMKK2 inhibitor herein refer to total amounts administered, that is, if more than one composition is administered, the effective dosage amounts of a CaMKK2 inhibitor corresponds to the total amount administered. The compositions can be administered as a single dose or as divided doses. For example, the composition may be administered two or more times separated by 4 hours, 6 hours, 8 hours, 12 hours, a day, two days, three days, four days, one week, two weeks, or by three or more weeks.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of’ and “consisting of’ those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

Mammalian CaMKK2 proteins are 66-68-kDa kinases including unique N- and C-terminal domains, a central Ser/Thr-directed kinase domain, and a regulatory domain composed of overlapping autoinhibitory and CaM-binding regions. CaMKK2 proteins are auto-inhibited by a sequence located immediately C-terminal to its catalytic domain, and Ca2+/CaM binding causes conformational changes that stimulate kinase activity. Once activated, CaMKK2 proteins can phosphorylate CaMKIV and CaMKI increasing their enzymatic activity. 5′ AMP-activated protein kinase α (AMPKα) is an additional substrate of CaMKK2 proteins, and silencing of CaMKK2 proteins in mammalian cells almost completely abolishes AMPK activation. Although CaMKK2 proteins can be detected in many areas of the brain, outside this organ the expression of CaMKK2 proteins is less clear. In the immune system, CaMKK2 proteins have been found exclusively in myeloid cells, including hematopoietic progenitors, peritoneal macrophages and bone marrow-derived macrophages. Genetic ablation of CaMKK2 proteins interferes with development and function of myeloid cells, and in turn has important effects on the inflammatory response.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

One embodiment provides a compound having the structure of Formula (I), or a pharmaceutically acceptable salt thereof,

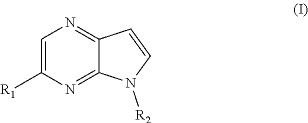

wherein,
R₁ is alkyl, cycloalkyl, optionally substituted aryl, optionally substituted heteroaryl, or amine; and
R₂ is optionally substituted aryl.

Another embodiment provides a compound of Formula (I), wherein R₁ is C3 cycloalkyl.

Another embodiment provides a compound of Formula (I), wherein R₁ is aryl optionally substituted by halogen, alkyl, or cycloalkyl. Another embodiment provides a compound of Formula (I), wherein R₁ is aryl optionally substituted by methyl. Another embodiment provides a compound of Formula (I), wherein R₁ is aryl optionally substituted by C3 cycloalkyl.

Another embodiment provides a compound of Formula (I), wherein R₁ is heteroaryl optionally substituted by halogen or alkyl. Another embodiment provides a compound of Formula (I), wherein R₁ is heteroaryl optionally substituted by methyl. Another embodiment provides a compound of Formula (I), wherein R₁ is bicyclic heteroaryl.

Another embodiment provides a compound of Formula (I), wherein R₂ is aryl optionally substituted by at least one substituent chosen from cycloalkyl, carbonyl, amine, —CN, heterocyclyl, heterocyclyloxy, heterocyclylalkyl or heteroaryl. Another embodiment provides a compound of Formula (I), wherein R₂ is aryl optionally substituted by cycloalkyl, wherein the cycloalkyl is C5-cycloalkyl. Another embodiment provides a compound of Formula (I), wherein R₂ is aryl optionally substituted by carbonyl, wherein the carbonyl is chosen from —C(═O)OH, —C(═O)NH—CN, or —C(═O)NH—SO₂—CF₃. Another embodiment provides a compound of Formula (I), wherein R₂ is aryl optionally substituted by amine, wherein the amine is chosen from —NH-alkyl, —NH-cycloalkyl, —NH— cycloalkylalkyl, or —NH-heterocyclyl. Another embodiment provides a compound of Formula (I), wherein R₂ is aryl optionally substituted by heterocyclyl, wherein the heterocyclyl is 5-6 membered heterocyclyl containing N. Another embodiment provides a compound of Formula (I), wherein R₂ is aryl optionally substituted by heteroaryl, wherein the heteroaryl is tetrazole.

One embodiment provides a compound having the structure of Formula (IIa), or a pharmaceutically acceptable salt thereof,

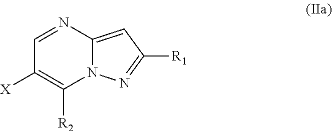

wherein,
R₁ is alkoxy, cycloalkyl, optionally substituted aryl, optionally substituted heteroaryl, or amine;
R₂ is optionally substituted aryl; and
X is hydrogen or halogen.

Another embodiment provides a compound of Formula (IIa), wherein R₁ is alkoxy chosen from methoxy or ethoxy. Another embodiment provides a compound of Formula (IIa), wherein R₁ is C3 cycloalkyl. Another embodiment provides a compound of Formula (IIa), wherein R₁ is aryl optionally substituted by halogen and alkoxy. Another embodiment provides a compound of Formula (IIa), wherein R₁ is aryl optionally substituted by methoxy. Another embodiment provides a compound of Formula (IIa), wherein R₁ is aryl optionally substituted by —O-cycloalkylalkyl. Another embodiment provides a compound of Formula (IIa), wherein R₁ is heteroaryl optionally substituted by methyl. Another embodiment provides a compound of Formula (IIa), wherein R₁ is pyrazole substituted by methyl.

Another embodiment provides a compound of Formula (IIa), wherein R₂ is aryl optionally substituted by at least one substituent chosen from carbonyl, alkoxy, or cycloalkyl. Another embodiment provides a compound of Formula (IIa), wherein R₂ is aryl optionally substituted by carbonyl, wherein the carbonyl is —C(═O)OH or —C(═O)-amine. Another embodiment provides a compound of Formula (IIa), wherein R₂ is aryl optionally substituted by alkoxy, wherein the alkoxy is methoxy. Another embodiment provides a compound of Formula (IIa), wherein R₂ is aryl optionally substituted by cycloalkyl, wherein the cycloalkyl is C5 cycloalkyl.

One embodiment provides a compound having the structure of Formula (IIb), or a pharmaceutically acceptable salt thereof,

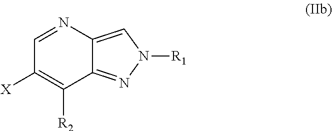

wherein,
R₁ is optionally substituted aryl;
R₂ is optionally substituted aryl; and
X is hydrogen or halogen.

Another embodiment provides a compound of Formula (IIb), wherein R₁ is aryl optionally substituted by at least one substituent chosen from halogen, alkyl, CF₃, alkoxy, —O—CF₃, or cycloalkyl. Another embodiment provides a compound of Formula (IIb), wherein R₁ is aryl optionally substituted by methyl. Another embodiment provides a compound of Formula (IIb), wherein R₁ is aryl optionally substituted by methoxy. Another embodiment provides a compound of Formula (IIb), wherein R₁ is aryl optionally substituted by C3-cycloalkyl.

Another embodiment provides a compound of Formula (IIb), wherein R₂ is aryl optionally substituted by at least one substituent chosen from carbonyl, alkoxy, amine, or cycloalkyl. Another embodiment provides a compound of Formula (IIb), wherein R₂ is aryl optionally substituted by carbonyl, wherein the carbonyl is chosen from —C(═O)OH or —C(═O)-amine. Another embodiment provides a compound of Formula (IIb), wherein R₂ is aryl optionally substituted by alkoxy, wherein the alkoxy is methoxy. Another embodiment provides a compound of Formula (IIb), wherein R₂ is aryl optionally substituted by amine, wherein the amine is —NH-cycloalkyl. Another embodiment provides a compound of Formula (IIb), wherein R₂ is aryl optionally substituted by cycloalkyl, wherein the cycloalkyl is C5 cycloalkyl.

One embodiment provides a compound having the structure of Formula (IIc), or a pharmaceutically acceptable salt thereof,

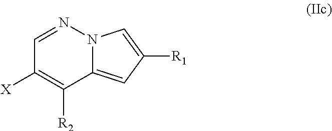

wherein,
R₁ is optionally substituted aryl;
R₂ is optionally substituted aryl; and
X is hydrogen or halogen.

Another embodiment provides a compound of Formula (IIc), wherein R₁ is aryl optionally substituted by at least one substituent chosen from halogen or alkyl. Another embodiment provides a compound of Formula (IIc), wherein R₁ is aryl optionally substituted by alkyl, wherein the alkyl is methyl.

Another embodiment provides a compound of Formula (IIc), wherein R₂ is aryl optionally substituted by at least one substituent chosen from carbonyl or cycloalkyl. Another embodiment provides a compound of Formula (IIc), wherein R₂ is aryl optionally substituted by carbonyl, wherein the carbonyl is —C(═O)OH. Another embodiment provides a compound of Formula (IIc), wherein R₂ is aryl optionally substituted by cycloalkyl, wherein the cycloalkyl is C5 cycloalkyl.

One embodiment provides a compound having the structure of Formula (III), or a pharmaceutically acceptable salt thereof,

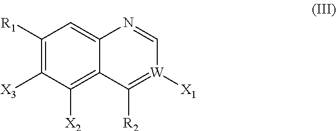

wherein
W is carbon or nitrogen;
R₁ is alkoxy, cyclylalkoxy, heterocyclyl, or heteroaryl;
R₂ is optionally substituted aryl; and
X₁, X₂, and X₃ is independently hydrogen or halogen.

Another embodiment provides a compound of Formula (III), wherein R₁ is ethoxy. Another embodiment provides a compound of Formula (III), wherein R₁ is C3 cyclylalkoxy. Another embodiment provides a compound of Formula (III), wherein R₁ is C5 heterocyclyl containing at least one nitrogen. Another embodiment provides a compound of Formula (III), wherein R₁ is C5 heteroaryl containing at least one nitrogen.

Another embodiment provides a compound of Formula (III), wherein R₂ is aryl optionally substituted by at least one substituent chosen from halogen, carbonyl, cycloalkyl, or heterocyclyl. Another embodiment provides a compound of Formula (III), wherein R₂ is aryl optionally substituted by carbonyl, wherein the carbonyl is —C(═O)OH. Another embodiment provides a compound of Formula (III), wherein R₂ is aryl optionally substituted by cycloalkyl, wherein the cycloalkyl is C3-C5 cycloalkyl. Another embodiment provides a compound of Formula (III), wherein R₂ is aryl optionally substituted by heterocyclyl, wherein the heterocyclyl is C3-C6 heterocyclyl containing at least one nitrogen.

One embodiment provides a pharmaceutical composition comprising a compound of Formula (I), or a pharmaceutically acceptable salt thereof, and a pharmaceutically acceptable excipient. One embodiment provides a pharmaceutical composition comprising a compound of Formula (IIa), or a pharmaceutically acceptable salt thereof, and a pharmaceutically acceptable excipient. One embodiment provides a pharmaceutical composition comprising a compound of Formula (IIb), or a pharmaceutically acceptable salt thereof, and a pharmaceutically acceptable excipient. One embodiment provides a pharmaceutical composition comprising a compound of Formula (IIc), or a pharmaceutically acceptable salt thereof, and a pharmaceutically acceptable excipient. One embodiment provides a pharmaceutical composition comprising a compound of Formula (III), or a pharmaceutically acceptable salt thereof, and a pharmaceutically acceptable excipient.

One embodiment provides a method of regulating gene transcription in a cell comprising inhibiting CaMKK2 activity by exposing the CaMKK2 enzyme to a compound of Formula (I). One embodiment provides a method of regulating gene transcription in a cell comprising inhibiting CaMKK2 activity by exposing the CaMKK2 enzyme to a compound of Formula (IIa). One embodiment provides a method of regulating gene transcription in a cell comprising inhibiting CaMKK2 activity by exposing the CaMKK2 enzyme to a compound of Formula (IIb). One embodiment provides a method of regulating gene transcription in a cell comprising inhibiting CaMKK2 activity by exposing the CaMKK2 enzyme to a compound of Formula (IIc). One embodiment provides a method of regulating gene transcription in a cell comprising inhibiting CaMKK2 activity by exposing the CaMKK2 enzyme to a compound of Formula (III).

One embodiment provides a method of treating cancer in a patient in need thereof, comprising administering to the patient a therapeutically effective amount of a compound of Formula (I), or a pharmaceutically acceptable salt thereof. One embodiment provides a method of treating cancer in a patient in need thereof, comprising administering to the patient a therapeutically effective amount of a compound of Formula (IIa), or a pharmaceutically acceptable salt thereof. One embodiment provides a method of treating cancer in a patient in need thereof, comprising administering to the patient a therapeutically effective amount of a compound of Formula (IIb), or a pharmaceutically acceptable salt thereof. One embodiment provides a method of treating cancer in a patient in need thereof, comprising administering to the patient a therapeutically effective amount of a compound of Formula (IIc), or a pharmaceutically acceptable salt thereof. One embodiment provides a method of treating cancer in a patient in need thereof, comprising administering to the patient a therapeutically effective amount of a compound of Formula (III), or a pharmaceutically acceptable salt thereof.

In some embodiments, the substituted pyrrolopyrazine derivative compound disclosed herein has the structure provided in Table 1.

TABLE 1

| Chemical | | |
|---|---|---|
| Synthesis | | |
| Example | Structure | Name |
| 1 | 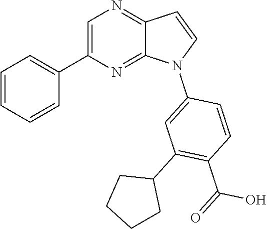 [SMILES: O=C(O)c1ccc(-n2ccc3ncc(-c4ccccc4)nc32)cc1C1CCCC1] | 2-Cyclopentyl-4-(3- phenylpyrrolo[2,3-b]pyrazin-5- yl)benzoic acid |
| 2 | 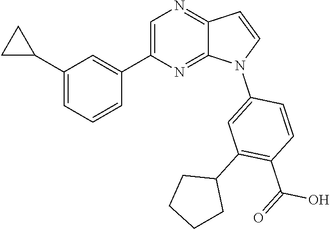 [SMILES: O=C(O)c1ccc(-n2ccc3ncc(-c4cccc(C5CC5)c4)nc32)cc1C1CCCC1] | 2-Cyclopentyl-4-[3-(3- cyclopropylphenyl)pyrrolo[2,3- b]pyrazin-5-yl]benzoic acid |
| 3 | 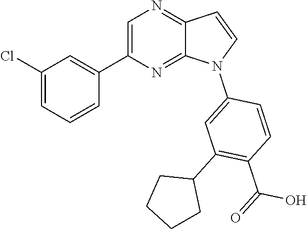 [SMILES: O=C(O)c1ccc(-n2ccc3ncc(-c4cccc(Cl)c4)nc32)cc1C1CCCC1] | 4-[3-(3- Chlorophenyl)pyrrolo[2,3- b]pyrazin-5-yl]-2-cyclopentyl- benzoic acid |
| 4 | 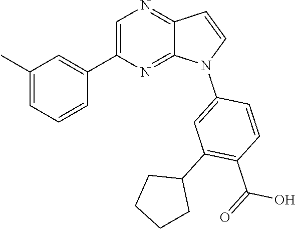 [SMILES: Cc1cccc(-c2cnc3ccn(-c4ccc(C(=O)O)c(C5CCCC5)c4)c3n2)c1] | 2-Cyclopentyl-4-(3-(m-tolyl)- 5H-pyrrolo[2,3-b]pyrazin-5- yl)benzoic acid |
| 5 | 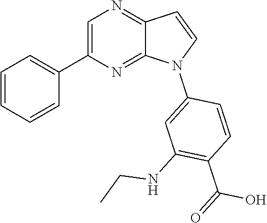 [SMILES: CCNc1cc(-n2ccc3ncc(-c4ccccc4)nc32)ccc1C(=O)O] | 2-(Ethylamino)-4-(3- phenylpyrrolo[2,3-b]pyrazin-5- yl)benzoic acid |
| 6 | 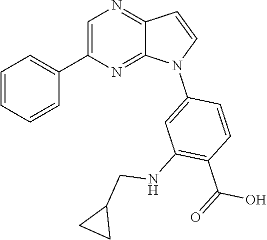 [SMILES: O=C(O)c1ccc(-n2ccc3ncc(-c4ccccc4)nc32)cc1NCC1CC1] | 2-((Cyclopropylmethyl)amino)- 4-(3-phenyl-5H-pyrrolo[2,3- b]pyrazin-5-yl)benzoic acid |
| 7 | 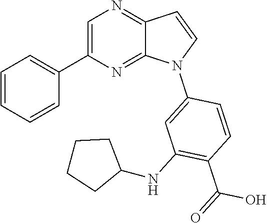 [SMILES: O=C(O)c1ccc(-n2ccc3ncc(-c4ccccc4)nc32)cc1NC1CCCC1] | 2-(Cyclopentylamino)-4-(3- phenyl-5H-pyrrolo[2,3- b]pyrazin-5-yl)benzoic acid |
| 8 | 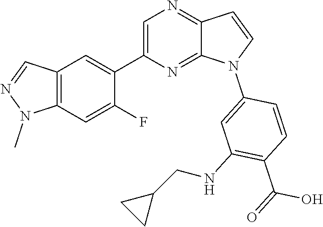 [SMILES: Cn1ncc2cc(-c3cnc4ccn(-c5ccc(C(=O)O)c(NCC6CC6)c5)c4n3)c(F)cc21] | 2-((Cyclopropylmethyl)amino)- 4-(3-(6-fluoro-1-methyl-1H- indazol-5-yl)-5H-pyrrolo[2,3- b]pyrazin-5-yl)benzoic acid |
| 9 | 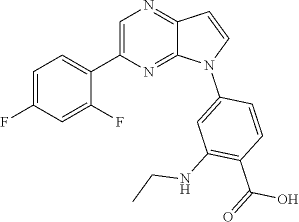 [SMILES: CCNc1cc(-n2ccc3ncc(-c4ccc(F)cc4F)nc32)ccc1C(=O)O] | 4-(3-(2,4-Difluorophenyl)-5H- pyrrolo[2,3-b]pyrazin-5-yl)-2- (ethylamino)benzoic acid |
| 10 | 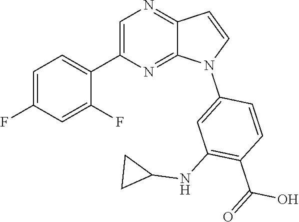 [SMILES: O=C(O)c1ccc(-n2ccc3ncc(-c4ccc(F)cc4F)nc32)cc1NC1CC1] | 2-(Cyclopropylamino)-4-(3-(2,4- difluorophenyl)-5H-pyrrolo[2,3- b]pyrazin-5-yl)benzoic acid |
| 11 | 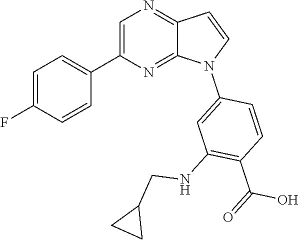 [SMILES: O=C(O)c1ccc(-n2ccc3ncc(-c4ccc(F)cc4)nc32)cc1NCC1CC1] | 2-((Cyclopropylmethyl)amino)- 4-(3-(4-fluorophenyl)-5H- pyrrolo[2,3-b]pyrazin-5- yl)benzoic acid |
| 12 | 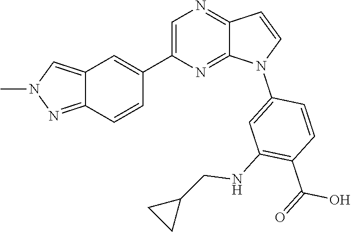 [SMILES: Cn1cc2cc(-c3cnc4ccn(-c5ccc(C(=O)O)c(NCC6CC6)c5)c4n3)ccc2n1] | 2-(Cyclopropylmethylamino)-4- (3-(2-methyl-2H-indazol-5-yl)- 5H-pyrrolo[2,3-b]pyrazin-5- yl)benzoic acid |
| 13 | 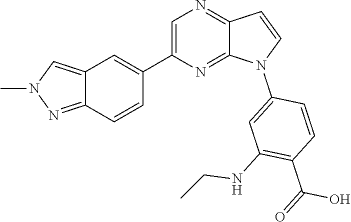 [SMILES: CCNc1cc(-n2ccc3ncc(-c4ccc5nn(C)cc5c4)nc32)ccc1C(=O)O] | 2-(Ethylamino)-4-(3-(2-methyl- 2H-indazol-5-yl)-5H- pyrrolo[2,3-b]pyrazin-5- yl)benzoic acid |
| 14 | 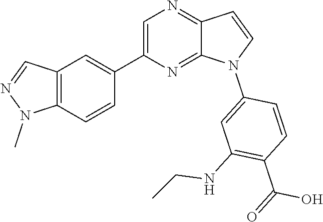 [SMILES: CCNc1cc(-n2ccc3ncc(-c4ccc5c(cnn5C)c4)nc32)ccc1C(=O)O] | 2-(Ethylamino)-4-(3-(1-methyl- 1H-indazol-5-yl)-5H- pyrrolo[2,3-b]pyrazin-5- yl)benzoic acid |
| 15 | 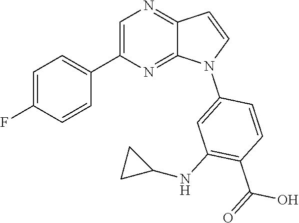 [SMILES: O=C(O)c1ccc(-n2ccc3ncc(-c4ccc(F)cc4)nc32)cc1NC1CC1] | 2-(Cyclopropylamino)-4-(3-(4- fluorophenyl)-5H-pyrrolo[2,3- b]pyrazin-5-yl)benzoic acid |
| 16 | 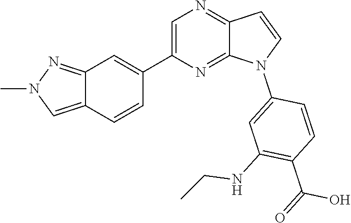 [SMILES: CCNc1cc(-n2ccc3ncc(-c4ccc5cn(C)nc5c4)nc32)ccc1C(=O)O] | 2-(Ethylamino)-4-(3-(2-methyl- 2H-indazol-6-yl)-5H- pyrrolo[2,3-b]pyrazin-5- yl)benzoic acid |
| 17 | 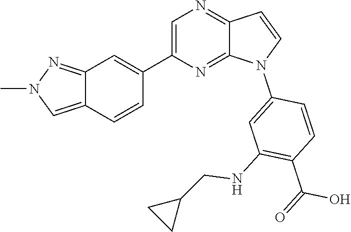 [SMILES: Cn1cc2ccc(-c3cnc4ccn(-c5ccc(C(=O)O)c(NCC6CC6)c5)c4n3)cc2n1] | 2-((Cyclopropylmethyl)amino)- 4-(3-(2-methyl-2H-indazol-6- yl)-5H-pyrrolo[2,3-b]pyrazin-5- yl)benzoic acid |
| 18 | 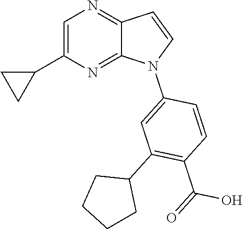 [SMILES: O=C(O)c1ccc(-n2ccc3ncc(C4CC4)nc32)cc1C1CCCC1] | 2-Cyclopentyl-4-(3-cyclopropyl- 5H-pyrrolo[2,3-b]pyrazin-5- yl)benzoic acid |
| 19 | 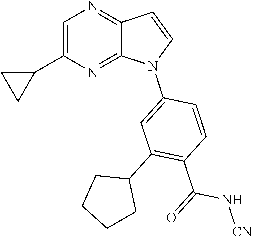 [SMILES: N#CNC(=O)c1ccc(-n2ccc3ncc(C4CC4)nc32)cc1C1CCCC1] | N-Cyano-2-cyclopentyl-4-(3- cyclopropyl-5H-pyrrolo[2,3- b]pyrazin-5-yl)benzamide |
| 20 | 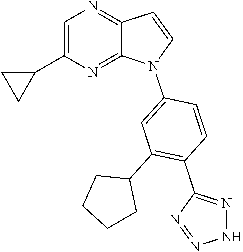 [SMILES: c1cc(-c2nn[nH]n2)c(C2CCCC2)cc1-n1ccc2ncc(C3CC3)nc21] | 5-(3-cyclopentyl-4-(2H-tetrazol- 5-yl)phenyl)-3-cyclopropyl-5H- pyrrolo[2,3-b]pyrazine |
| 21 | 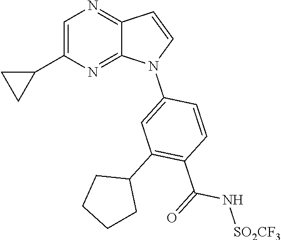 | 2-Cyclopentyl-4-(3-cyclopropyl- 5H-pyrrolo[2,3-b]pyrazin-5-yl)- N- ((trifluoromethyl)sulfonyl) benzamide |
| 22 | 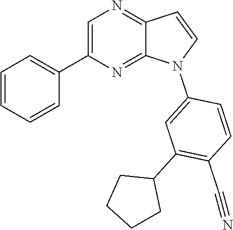 [SMILES: N#Cc1ccc(-n2ccc3ncc(-c4ccccc4)nc32)cc1C1CCCC1] | 2-Cyclopentyl-4-(3-phenyl-5H- pyrrolo[2,3-b]pyrazin-5- yl)benzonitrile |
| 23 | 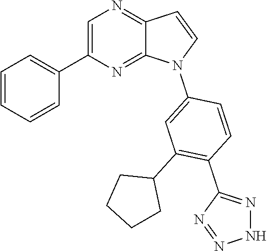 [SMILES: c1ccc(-c2cnc3ccn(-c4ccc(-c5nn[nH]n5)c(C5CCCC5)c4)c3n2)cc1] | 5-(3-Cyclopentyl-4-(2H-tetrazol- 5-yl)phenyl)-3-phenyl-5H- pyrrolo[2,3-b]pyrazine |
| 24 | 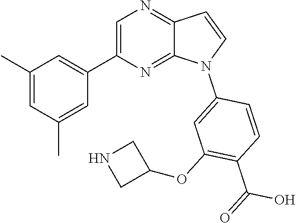 [SMILES: Cc1cc(C)cc(-c2cnc3ccn(-c4ccc(C(=O)O)c(OC5CNC5)c4)c3n2)c1] | 2-(azetidin-3-yloxy)-4-(3- (3,5-dimethylphenyl)-5H- pyrrolo[2,3-b]pyrazin-5- yl)benzoic acid |
| 25 | 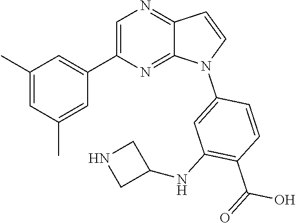 [SMILES: Cc1cc(C)cc(-c2cnc3ccn(-c4ccc(C(=O)O)c(NC5CNC5)c4)c3n2)c1] | 2-(azetidin-3-ylamino)-4-(3- (3,5-dimethylphenyl)-5H- pyrrolo[2,3-b]pyrazin-5- yl)benzoic acid |
| 26 | 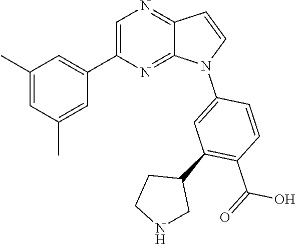 | (R) and (S)-4-(3-(3,5- dimethylphenyl)-5H- pyrrolo[2,3-b]pyrazin-5-yl)- 2-(pyrrolidin-3-yl) benzoic acid |
| | 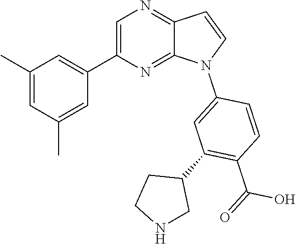 | |
| 27 | 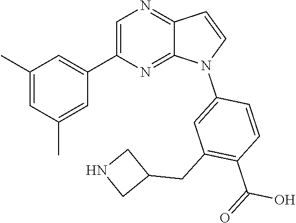 [SMILES: Cc1cc(C)cc(-c2cnc3ccn(-c4ccc(C(=O)O)c(CC5CNC5)c4)c3n2)c1] | 2-(azetidin-3-ylmethyl)-4-(3- (3,5-dimethylphenyl)-5H- pyrrolo[2,3-b] pyrazin-5-yl) benzoic acid |
| 28 | 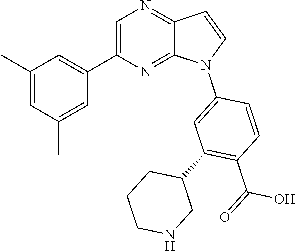 | (R) and (S)-4-(3-(3,5- dimethylphenyl)-5H- pyrrolo[2,3-b]pyrazin-5-yl)-2- (piperidin-3-yl) benzoic acid |
| | 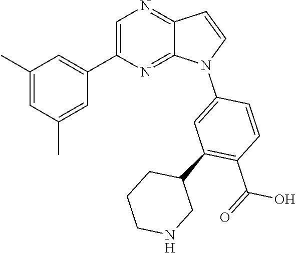 | |
| 29 | 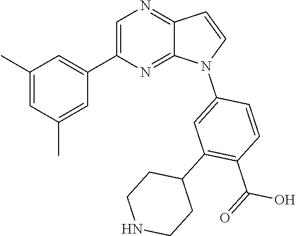 [SMILES: Cc1cc(C)cc(-c2cnc3ccn(-c4ccc(C(=O)O)c(C5CCNCC5)c4)c3n2)c1] | 4-(3-(3,5-dimethylphenyl)- 5H-pyrrolo[2,3-b]pyrazin-5- yl)-2-(piperidin-4-yl)benzoic acid |
| 30 | 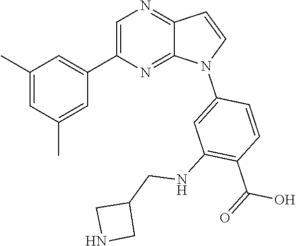 [SMILES: Cc1cc(C)cc(-c2cnc3ccn(-c4ccc(C(=O)O)c(NCC5CNC5)c4)c3n2)c1] | 2-((azetidin-3- ylmethyl)amino)-4-(3-(3,5- dimethylphenyl)-5H- pyrrolo[2,3-b]pyrazin-5- yl)benzoic acid |
| 31 | 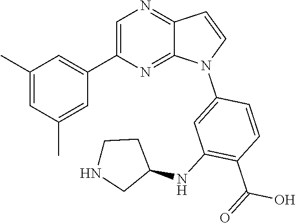 [SMILES: Cc1cc(C)cc(-c2cnc3ccn(-c4ccc(C(=O)O)c(N[C@@H]5CCNC5)c4)c3n2)c1] | (R)-4-(3-(3,5- dimethylphenyl)-5H- pyrrolo[2,3-b]pyrazin-5-yl)-2- (pyrrolidin-3-ylamino)benzoic acid |
| 32 | 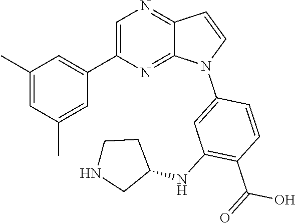 [SMILES: Cc1cc(C)cc(-c2cnc3ccn(-c4ccc(C(=O)O)c(N[C@H]5CCNC5)c4)c3n2)c1] | (S)-4-(3-(3,5- dimethylphenyl)-5H- pyrrolo[2,3-b]pyrazin-5-yl)-2- (pyrrolidin-3-ylamino)benzoic acid |
| 33 | 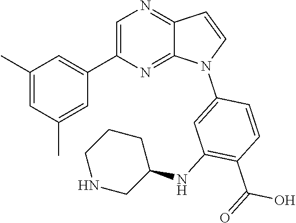 [SMILES: Cc1cc(C)cc(-c2cnc3ccn(-c4ccc(C(=O)O)c(N[C@@H]5CCCNC5)c4)c3n2)c1] | (R)-4-(3-(3,5- dimethylphenyl)-5H- pyrrolo[2,3-b]pyrazin-5-yl)-2- (piperidin-3-ylamino)benzoic acid |
| 34 | 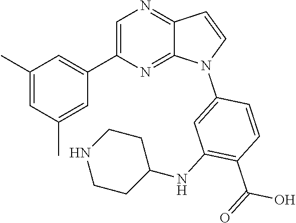 [SMILES: Cc1cc(C)cc(-c2cnc3ccn(-c4ccc(C(=O)O)c(NC5CCNCC5)c4)c3n2)c1] | 4-(3-(3,5-dimethylphenyl)- 5H-pyrrolo[2,3-b]pyrazin-5- yl)-2-(piperidin-4- ylamino)benzoic acid |
| 35 | 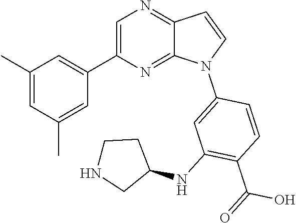 [SMILES: Cc1cc(C)cc(-c2cnc3ccn(-c4ccc(C(=O)O)c(N[C@@H]5CCNC5)c4)c3n2)c1] | (R)-4-(3-(3,5- dimethylphenyl)-5H- pyrrolo[2,3-b]pyrazin-5-yl)-2- (pyrrolidin-3-yloxy)benzoic acid |

In some embodiments, the substituted pyrazolopyrimidine derivative compound disclosed herein has the structure provided in Table 2.

TABLE 2

| Chemical | | |
|---|---|---|
| Synthesis | | |
| Example | Structure | Name |
| 2.1 | 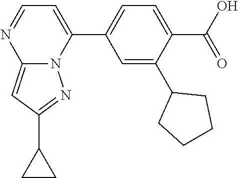 [SMILES: O=C(O)c1ccc(-c2ccnc3cc(C4CC4)nn23)cc1C1CCCC1] | 2-cyclopentyl-4-(2- cyclopropylpyrazolo[1,5-a]pyrimidin-7- yl)benzoic acid |
| 2.2 | 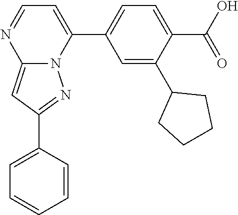 [SMILES: O=C(O)c1ccc(-c2ccnc3cc(-c4ccccc4)nn23)cc1C1CCCC1] | 2-cyclopentyl-4-(2-phenylpyrazolo[1,5- a]pyrimidin-7-yl)benzoic acid |
| 2.3 | 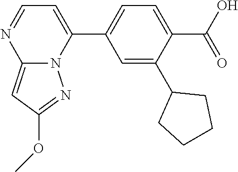 [SMILES: COc1cc2nccc(-c3ccc(C(=O)O)c(C4CCCC4)c3)n2n1] | 2-cyclopentyl-4-(2-methoxypyrazolo[1,5- a]pyrimidin-7-yl)benzoic acid |
| 2.4 | 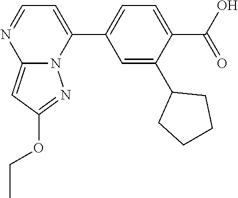 [SMILES: CCOc1cc2nccc(-c3ccc(C(=O)O)c(C4CCCC4)c3)n2n1] | 2-cyclopentyl-4-(2-ethoxypyrazolo[1,5- a]pyrimidin-7-yl)benzoic acid |
| 2.5 | 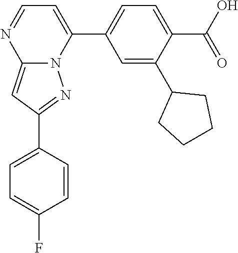 [SMILES: O=C(O)c1ccc(-c2ccnc3cc(-c4ccc(F)cc4)nn23)cc1C1CCCC1] | 2-cyclopentyl-4-(2-(4- fluorophenyl)pyrazolo[1,5-a]pyrimidin-7- yl)benzoic acid |
| 2.6 | 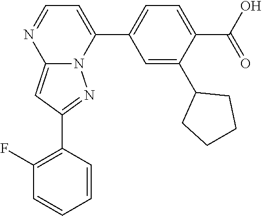 [SMILES: O=C(O)c1ccc(-c2ccnc3cc(-c4ccccc4F)nn23)cc1C1CCCC1] | 2-cyclopenty 1-4-(2-(2- fluorophenyl)pyrazolo[1,5-a]pyrimidin-7- yl)benzoic acid |
| 2.7 | 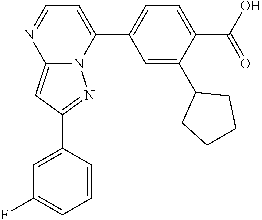 [SMILES: O=C(O)c1ccc(-c2ccnc3cc(-c4cccc(F)c4)nn23)cc1C1CCCC1] | 2-cyclopentyl-4-(2-(3- fluorophenyl)pyrazolo[1,5-a]pyrimidin-7- yl)benzoic acid |
| 2.8 | 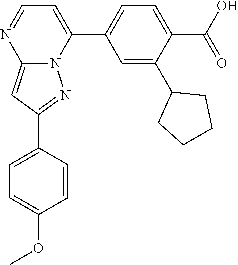 [SMILES: COc1ccc(-c2cc3nccc(-c4ccc(C(=O)O)c(C5CCCC5)c4)n3n2)cc1] | 2-cyclopentyl-4-(2-(4- methoxyphenyl)pyrazolo[1,5-a]pyrimidin- 7-yl)benzoic acid |
| 2.9 | 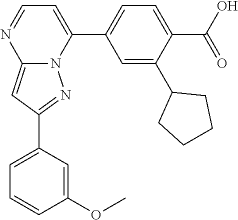 [SMILES: COc1cccc(-c2cc3nccc(-c4ccc(C(=O)O)c(C5CCCC5)c4)n3n2)c1] | 2-cyclopentyl-4-(2-(3- methoxyphenyl)pyrazolo[1,5-a]pyrimidin- 7-yl)benzoic acid |
| 2.10 | 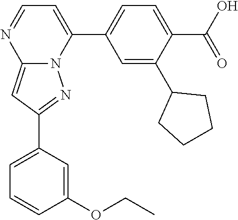 [SMILES: CCOc1cccc(-c2cc3nccc(-c4ccc(C(=O)O)c(C5CCCC5)c4)n3n2)c1] | 2-cyclopentyl-4-(2-(3- ethoxyphenyl)pyrazolo[1,5-a]pyrimidin-7- yl)benzoic acid |
| 2.11 | 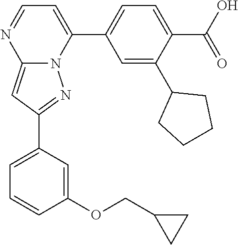 [SMILES: O=C(O)c1ccc(-c2ccnc3cc(-c4cccc(OCC5CC5)c4)nn23)cc1C1CCCC1] | 2-cyclopentyl-4-(2-(3- (cyclopropylmethoxy)phenyl)pyrazolo[1,5- a]pyrimidin-7-yl)benzoic acid |
| 2.12 | 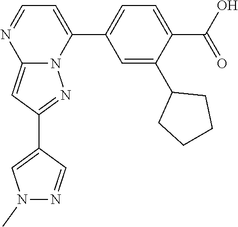 [SMILES: Cn1cc(-c2cc3nccc(-c4ccc(C(=O)O)c(C5CCCC5)c4)n3n2)cn1] | 2-cyclopentyl-4-(2-(1-methyl-1H-pyrazol- 4-yl)pyrazolo[1,5-a]pyrimidin-7- yl)benzoic acid |
| 2.13 | 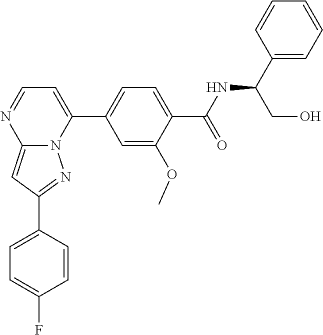 [SMILES: COc1cc(-c2ccnc3cc(-c4ccc(F)cc4)nn23)ccc1C(=O)N[C@H](CO)c1ccccc1] | (S)-4-(2-(4-fluorophenyl)pyrazolo[1,5- a]pyrimidin-7-yl)-N-(2-hydroxy-1- phenylethyl)-2-methoxybenzamide |

In some embodiments, the substituted pyrazolopyridine derivative compound disclosed herein has the structure provided in Table 3.

TABLE 3

| Chemical | | |
|---|---|---|
| Synthesis | | |
| Example | Structure | Name |
| 3.1 | 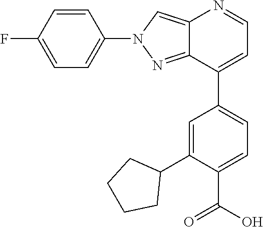 [SMILES: O=C(O)c1ccc(-c2ccnc3cn(-c4ccc(F)cc4)nc23)cc1C1CCCC1] | 2-Cyclopentyl-4-[2-(4- fluorophenyl)pyrazolo[4,3-b]pyridin-7- yl]benzoic acid |
| 3.2 | 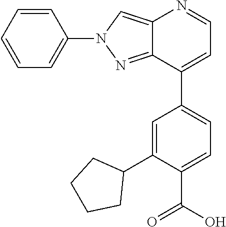 [SMILES: O=C(O)c1ccc(-c2ccnc3cn(-c4ccccc4)nc23)cc1C1CCCC1] | 2-Cyclopentyl-4-(2-phenyl-2H- pyrazolo[4,3-b]pyridin-7-yl)benzoic acid |
| 3.3 | 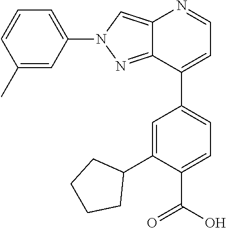 [SMILES: Cc1cccc(-n2cc3nccc(-c4ccc(C(=O)O)c(C5CCCC5)c4)c3n2)c1] | 2-Cyclopentyl-4-(2-(m-tolyl)-2H- pyrazolo[4,3-b]pyridin-7-yl)benzoic acid |
| 3.4 | 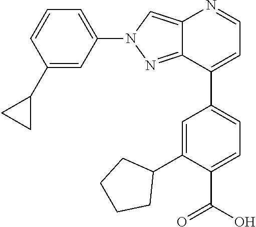 [SMILES: O=C(O)c1ccc(-c2ccnc3cn(-c4cccc(C5CC5)c4)nc23)cc1C1CCCC1] | 2-Cyclopentyl-4-(2-(3-cyclopropylphenyl)- 2H-pyrazolo[4,3-b]pyridin-7-yl)benzoic acid |
| 3.5 | 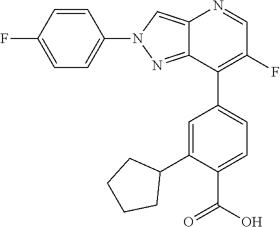 [SMILES: O=C(O)c1ccc(-c2c(F)cnc3cn(-c4ccc(F)cc4)nc23)cc1C1CCCC1] | 2-Cyclopentyl-4-(6-fluoro-2-(4- fluorophenyl)-2H-pyrazolo[4,3-b]pyridin- 7-yl)benzoic acid |
| 3.6 | 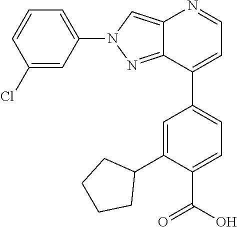 [SMILES: O=C(O)c1ccc(-c2ccnc3cn(-c4cccc(Cl)c4)nc23)cc1C1CCCC1] | 4-(2-(3-Chlorophenyl)-2H-pyrazolo[4,3- b]pyridin-7-yl)-2-cyclopentylbenzoic acid |
| 3.7 | 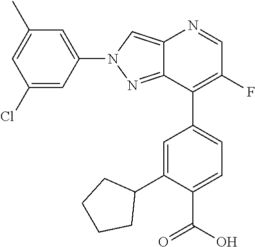 [SMILES: Cc1cc(Cl)cc(-n2cc3ncc(F)c(-c4ccc(C(=O)O)c(C5CCCC5)c4)c3n2)c1] | 4-(2-(3-Chloro-5-methylphenyl)-6-fluoro- 2H-pyrazolo[4,3-b]pyridin-7-yl)-2- cyclopentylbenzoic acid |
| 3.8 | 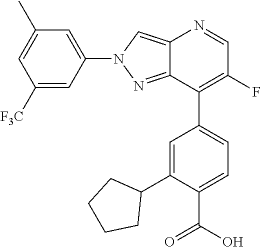 [SMILES: Cc1cc(-n2cc3ncc(F)c(-c4ccc(C(=O)O)c(C5CCCC5)c4)c3n2)cc(C(F)(F)F)c1] | 2-Cyclopentyl-4-(6-fluoro-2-(3-methyl-5- (trifluoromethyl)phenyl)-2H-pyrazolo[4,3- b]pyridin-7-yl)benzoic acid |
| 3.9 | 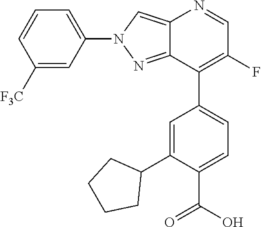 [SMILES: O=C(O)c1ccc(-c2c(F)cnc3cn(-c4cccc(C(F)(F)F)c4)nc23)cc1C1CCCC1] | 2-Cyclopentyl-4-(6-fluoro-2-(3- (trifluoromethyl)phenyl)-2H-pyrazolo[4,3- b]pyridin-7-yl)benzoic acid |
| 3.10 | 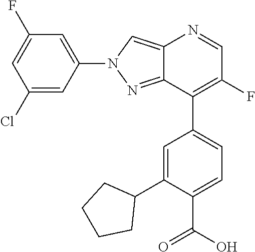 [SMILES: O=C(O)c1ccc(-c2c(F)cnc3cn(-c4cc(F)cc(Cl)c4)nc23)cc1C1CCCC1] | 4-(2-(3-Chloro-5-fluorophenyl)-6-fluoro- 2H-pyrazolo[4,3-b]pyridin-7-yl)-2- cyclopentylbenzoic acid |
| 3.11 | 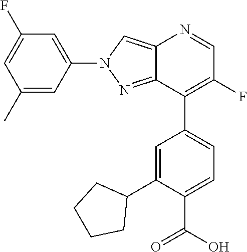 [SMILES: Cc1cc(F)cc(-n2cc3ncc(F)c(-c4ccc(C(=O)O)c(C5CCCC5)c4)c3n2)c1] | 2-Cyclopentyl-4-(6-fluoro-2-(3-fluoro-5- methylphenyl)-2H-pyrazolo[4,3-b]pyridin- 7-yl)benzoic acid |
| 3.12 | 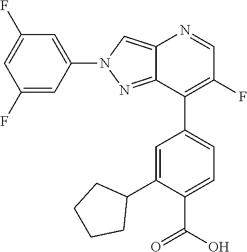 [SMILES: O=C(O)c1ccc(-c2c(F)cnc3cn(-c4cc(F)cc(F)c4)nc23)cc1C1CCCC1] | 2-Cyclopentyl-4-(2-(3,5-difluorophenyl)-6- fluoro-2H-pyrazolo[4,3-b]pyridin-7- yl)benzoic acid |
| 3.13 | 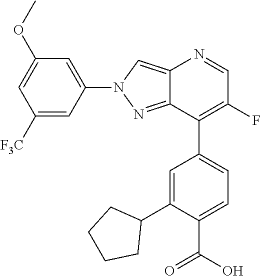 [SMILES: COc1cc(-n2cc3ncc(F)c(-c4ccc(C(=O)O)c(C5CCCC5)c4)c3n2)cc(C(F)(F)F)c1] | 2-Cyclopentyl-4-(6-fluoro-2-(3-methoxy- 5-(trifluoromethyl)phenyl)-2H- pyrazolo[4,3-b]pyridin-7-yl)benzoic acid |
| 3.14 | 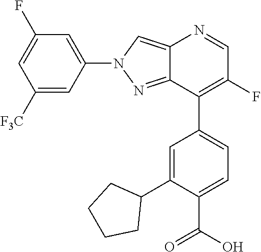 [SMILES: O=C(O)c1ccc(-c2c(F)cnc3cn(-c4cc(F)cc(C(F)(F)F)c4)nc23)cc1C1CCCC1] | 2-Cyclopentyl-4-(6-fluoro-2-(3-fluoro-5- (trifluoromethyl)phenyl)-2H-pyrazolo[4,3- b]pyridin-7-yl)benzoic acid |
| 3.15 | 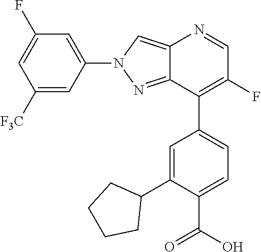 [SMILES: O=C(O)c1ccc(-c2c(F)cnc3cn(-c4cc(F)cc(C(F)(F)F)c4)nc23)cc1C1CCCC1] | 4-(2-(3-Chloro-5-(trifluoromethyl)phenyl)- 6-fluoro-2H-pyrazolo[4,3-b]pyridin-7-yl)- 2-cyclopentylbenzoic |
| 3.16 | 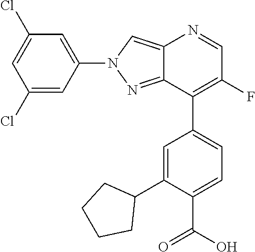 [SMILES: O=C(O)c1ccc(-c2c(F)cnc3cn(-c4cc(Cl)cc(Cl)c4)nc23)cc1C1CCCC1] | 2-Cyclopentyl-4-(2-(3,5-dichlorophenyl)- 6-fluoro-2H-pyrazolo[4,3-b]pyridin-7- yl)benzoic acid |
| 3.17 | 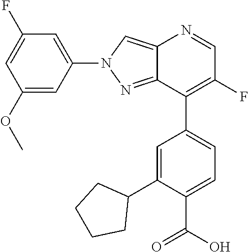 [SMILES: COc1cc(F)cc(-n2cc3ncc(F)c(-c4ccc(C(=O)O)c(C5CCCC5)c4)c3n2)c1] | 2-Cyclopentyl-4-(6-fluoro-2-(3-fluoro-5- methoxyphenyl)-2H-pyrazolo[4,3- b]pyridin-7-yl)benzoic acid |
| 3.18 | 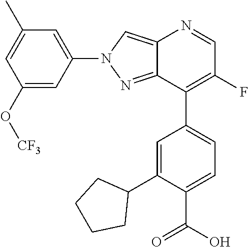 [SMILES: Cc1cc(OC(F)(F)F)cc(-n2cc3ncc(F)c(-c4ccc(C(=O)O)c(C5CCCC5)c4)c3n2)c1] | 2-Cyclopentyl-4-(6-fluoro-2-(3-methyl-5- (trifluoromethoxy)phenyl)-2H- pyrazolo[4,3-b]pyridin-7-yl)benzoic acid |
| 3.19 | 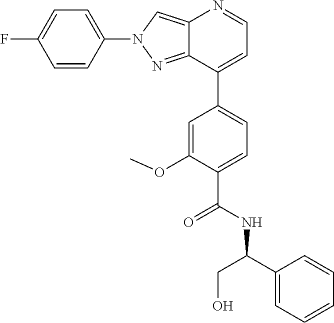 [SMILES: COc1cc(-c2ccnc3cn(-c4ccc(F)cc4)nc23)ccc1C(=O)N[C@H](CO)c1ccccc1] | (S)-4-(2-(4-Fluorophenyl)-2H- pyrazolo[4,3-b]pyridin-7-yl)-N-(2- hydroxy-1-phenylethyl)-2- methoxy benzamide |
| 3.20 | 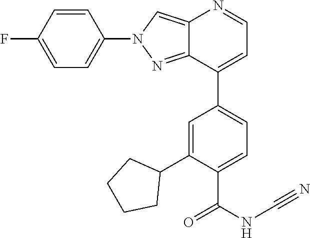 [SMILES: N#CNC(=O)c1ccc(-c2ccnc3cn(-c4ccc(F)cc4)nc23)cc1C1CCCC1] | N-Cyano-2-cyclopentyl-4-(2-(4- fluorophenyl)-2H-pyrazolo[4,3-b]pyridin- 7-yl)benzamide |
| 3.21 | 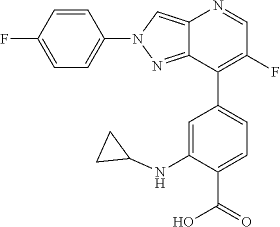 [SMILES: O=C(O)c1ccc(-c2c(F)cnc3cn(-c4ccc(F)cc4)nc23)cc1NC1CC1] | 2-(Cyclopropylamino)-4-(6-fluoro-2-(4- fluorophenyl)-2H-pyrazolo[4,3-b]pyridin- 7-yl)benzoic acid |

In some embodiments, the substituted pyrrolopyridazine derivative compound disclosed herein has the structure provided in Table 4.

TABLE 4

| Chemical | | |
|---|---|---|
| Synthesis | | |
| Example | Structure | Name |
| 4.1 | 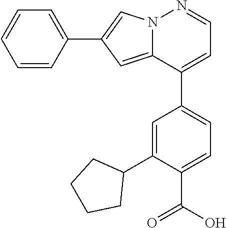 [SMILES: O=C(O)c1ccc(-c2ccnn3cc(-c4ccccc4)cc23)cc1C1CCCC1] | 2-Cyclopentyl-4-(6-phenylpyrrolo[1,2- b]pyridazin-4-yl)benzoic acid |
| 4.2 | 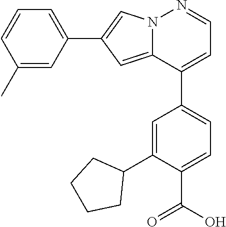 [SMILES: Cc1cccc(-c2cc3c(-c4ccc(C(=O)O)c(C5CCCC5)c4)ccnn3c2)c1] | 2-Cyclopentyl-4-[6-(m-tolyl)pyrrolo[1,2- b]pyridazin-4-yl]benzoic acid |
| 4.3 | 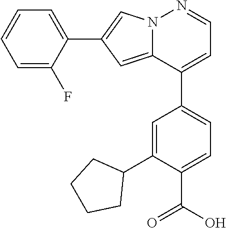 [SMILES: O=C(O)c1ccc(-c2ccnn3cc(-c4ccccc4F)cc23)cc1C1CCCC1] | 2-Cyclopentyl-4-[6-(2- fluorophenyl)pyrrolo[1,2-b]pyridazin-4- yl]benzoic acid |

In some embodiments, the substituted quinazoline or quinoline derivative compound disclosed herein has the structure provided in Table 5.

TABLE 5

| Chemical | | |
|---|---|---|
| Synthesis | | |
| Example | Structure | Name |
| 5.1 | 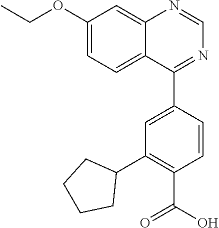 [SMILES: CCOc1ccc2c(-c3ccc(C(=O)O)c(C4CCCC4)c3)ncnc2c1] | 2-cyclopentyl-4-(7-ethoxy quinazolin-4- yl)benzoic acid |
| 5.2 | 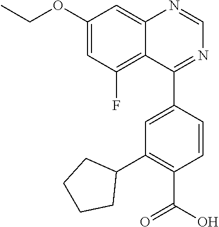 [SMILES: CCOc1cc(F)c2c(-c3ccc(C(=O)O)c(C4CCCC4)c3)ncnc2c1] | 2-cyclopentyl-4-(7-ethoxy-5- fluoroquinazolin-4-yl)benzoic acid |
| 5.3 | 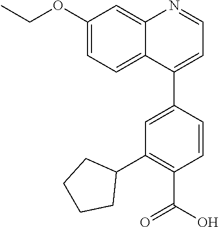 [SMILES: CCOc1ccc2c(-c3ccc(C(=O)O)c(C4CCCC4)c3)ccnc2c1] | 2-cyclopentyl-4-(7-ethoxy quinolin-4- yl)benzoic acid |
| 5.4 | 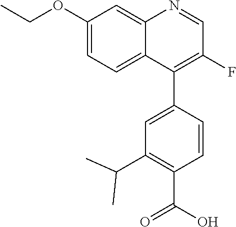 [SMILES: CCOc1ccc2c(-c3ccc(C(=O)O)c(C(C)C)c3)c(F)cnc2c1] | 4-(7-ethoxy-3-fluoroquinolin-4-yl)-2- isopropylbenzoic acid |
| 5.5 | 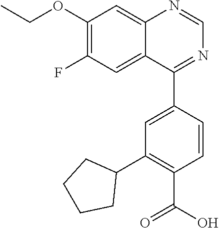 [SMILES: CCOc1cc2ncnc(-c3ccc(C(=O)O)c(C4CCCC4)c3)c2cc1F] | 2-cyclopentyl-4-(7-ethoxy-6- fluoroquinazolin-4-yl)benzoic acid |
| 5.6 | 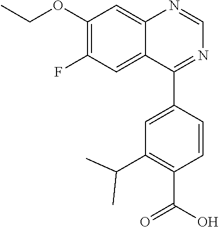 [SMILES: CCOc1cc2ncnc(-c3ccc(C(=O)O)c(C(C)C)c3)c2cc1F] | 4-(7-ethoxy-6-fluoroquinazolin-4-yl)-2- isopropylbenzoic acid |
| 5.7 | 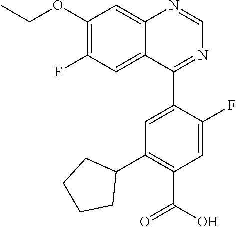 [SMILES: CCOc1cc2ncnc(-c3cc(C4CCCC4)c(C(=O)O)cc3F)c2cc1F] | 2-cyclopentyl-4-(7-ethoxy-6- fluoroquinazolin-4-yl)-5-fluorobenzoic acid |
| 5.8 | 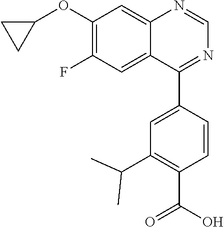 [SMILES: CC(C)c1cc(-c2ncnc3cc(OC4CC4)c(F)cc23)ccc1C(=O)O] | 4-(7-cyclopropoxy-6-fluoroquinazolin-4- yl)-2-isopropylbenzoic acid |
| 5.9 | 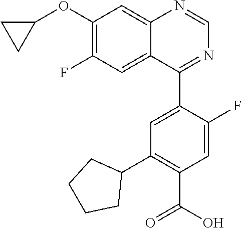 [SMILES: O=C(O)c1cc(F)c(-c2ncnc3cc(OC4CC4)c(F)cc23)cc1C1CCCC1] | 2-cyclopentyl-4-(7-cyclopropoxy-6- fluoroquinazolin-4-yl)-5-fluorobenzoic acid |
| 5.10 | 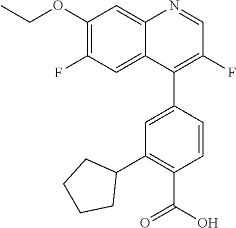 [SMILES: CCOc1cc2ncc(F)c(-c3ccc(C(=O)O)c(C4CCCC4)c3)c2cc1F] | 2-cyclopentyl-4-(7-ethoxy-3,6- difluoroquinolin-4-yl)benzoic acid |
| 5.11 | 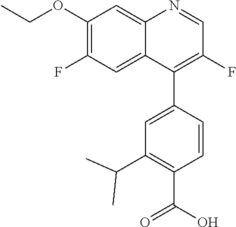 [SMILES: CCOc1cc2ncc(F)c(-c3ccc(C(=O)O)c(C(C)C)c3)c2cc1F] | 4-(7-ethoxy-3,6-difluoroquinolin-4-yl)-2- isopropylbenzoic acid |
| 5.12 | 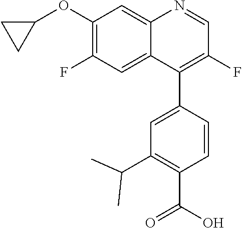 [SMILES: CC(C)c1cc(-c2c(F)cnc3cc(OC4CC4)c(F)cc23)ccc1C(=O)O] | 4-(7-cyclopropoxy-3,6-difluoroquinolin-4- yl)-2-isopropylbenzoic acid |

### EXAMPLE

The following examples are meant only to be illustrative and are not meant as limitations on the scope of the invention or of the appended claims.

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted. Spectra are given in ppm (δ) and coupling constants (J) are reported in Hertz. For ¹H NMR spectra, the solvent peak was used as the reference peak.

### Example 1: 2-Cyclopentyl-4-(3-phenylpyrrolo[2,3-b]pyrazin-5-yl)benzoic Acid

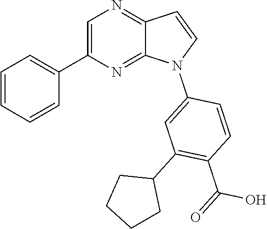 [SMILES: O=C(O)c1ccc(-n2ccc3ncc(-c4ccccc4)nc32)cc1C1CCCC1]

Step A. To a stirred solution of 3-chloro-5H-pyrrolo[2,3-b]pyrazine (3 g, 19.5 mmol) in DMF (30 mL) and water (6 mL) was added phenylboronic acid (3.57 g, 29.3 mmol), Pd(dppf)Cl₂ (300 mg, 0.41 mmol) and Cs₂CO₃ (14.1 g, 39.1 mmol). The resulting mixture purged with N₂ and allowed to stir at 100° C. for 3 h. Upon completion, the solvent was concentrated in vacuo and the residue taken up in water and extracted with ethyl acetate (3×). The combined organic layers were successively washed with brine, dried over anhydrous sodium sulfate, filtered and concentrated in vacuo. The residue was purified by a silica gel column chromatography (PE/A=4:1 as eluent) to obtain 3-phenyl-5H-pyrrolo[2,3-b]pyrazine as a yellow solid (3.6 g, 94.3%). [M+H] Calc'd for C₁₂H₉N₃, 196. Found 196.

Step B. To a stirred solution of 3-phenyl-5H-pyrrolo[2,3-b]pyrazine (400 mg, 2.1 mmol) in 1,4-dioxane (15 mL) was added methyl 4-bromo-2-cyclopentyl-benzoate (696 mg, 2.5 mmol), CuI (78 mg, 0.41 mmol), dimethylcyclohexane-1,2-diamine (116 mg, 0.82 mmol) and K₃PO₄ (868 mg, 4.1 mmol). The reaction was purged with N₂ and stirred at 110° C. for 2 h. Upon completion the reaction mixture was filtered and concentrated in vacuo. The crude product was purified by silica gel chromatography (10-100% gradient of ethyl acetate in petroleum ether) to obtain methyl 2-cyclopentyl-4-(3-phenylpyrrolo[2,3-b]pyrazin-5-yl)benzoate (600 mg, 73.7%). [M+H] Calc'd for C₂₅H₂₃N₃O₂, 398. Found 398.

Step C. To a stirred solution of methyl 2-cyclopentyl-4-(3-phenylpyrrolo[2,3-b]pyrazin-5-yl)benzoate (200 mg, 0.50 mmol) in methanol (3 mL), THF (3 mL) and water (1.2 mL) was added NaOH (200 mg, 5 mmol). The reaction was warmed to 50° C. overnight. Upon completion, the reaction mixture was concentrated in vacuo and taken up in water. The pH was adjusted to ˜3, and the precipitate filtered. The solid was further purified by Prep-HPLC to afford the title compound (50.4 mg, 26%) as a white solid. [M+H] Calc'd for C₂₄H₂₁N₃O₂, 384. Found 384. ¹H NMR (400 MHz, DMSO-d₆) δ 9.18 (s, 1H), 8.47 (d, J=3.9 Hz, 1H), 8.27 (d, J=2.2 Hz, 1H), 8.25-8.19 (m, 2H), 7.79 (d, J=8.4 Hz, 1H), 7.73 (dd, J=8.4, 2.2 Hz, 1H), 7.52 (dd, J=8.3, 6.4 Hz, 2H), 7.51-7.42 (m, 1H), 6.96 (d, J=3.8 Hz, 1H), 4.01-3.93 (m, 1H), 2.17-2.06 (m, 2H), 1.87-1.78 (m, 2H), 1.78-1.60 (m, 4H). Prep-HPLC conditions [Column: XBridge Prep C18 OBD Column, 5 um, 19*150 mm; Mobile Phase A: Water (10 mmol/L, NH₄HCO₃), Mobile Phase B: ACN; Flow rate: 25 mL/min; Gradient: 10% B to 60% B in 7.5 min; 254/210 nm; Rt: 4.9; 6.02 min]

### Example 2: 2-Cyclopentyl-4-[3-(3-cyclopropylphenyl)pyrrolo[2,3-b]pyrazin-5-yl]benzoic Acid

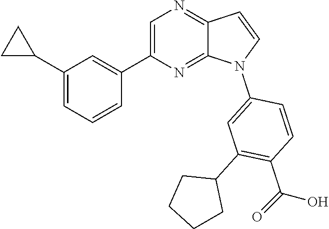 [SMILES: O=C(O)c1ccc(-n2ccc3ncc(-c4cccc(C5CC5)c4)nc32)cc1C1CCCC1]

The title compound was prepared in 34.3% overall yield as an off-white solid according to the preparation of Example 1 using 2-(3-cyclopropylphenyl)-4,4,5,5-tetramethyl-1,3,2-dioxaborolane in STEP A. [M+H] Calc'd for C₂₇H₂₅N₃O₂, 424. Found 424. ¹H NMR (400 MHz, DMSO-d₆) δ 9.18 (s, 1H), 8.45 (d, J=3.9 Hz, 1H), 8.23 (d, J=2.2 Hz, 1H), 8.01-7.92 (m, 2H), 7.75 (d, J=8.4 Hz, 1H), 7.69 (dd, J=8.2, 2.1 Hz, 1H), 7.40 (t, J=7.7 Hz, 1H), 7.14 (d, J=8.1 Hz, 1H), 6.96 (d, J=3.9 Hz, 1H), 3.92 (d, J=8.6 Hz, 1H), 2.13-2.10 (m, 2H), 2.10-1.97 (m, 1H), 1.82 (s, 2H), 1.68 (s, 4H), 1.05-0.96 (m, 2H), 0.83-0.74 (m, 2H).

### Example 3: 4-[3-(3-Chlorophenyl)pyrrolo[2,3-b]pyrazin-5-yl]-2-cyclopentyl-benzoic Acid

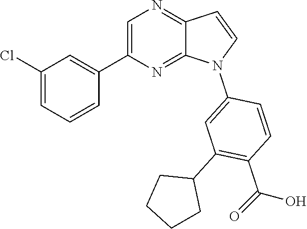 [SMILES: O=C(O)c1ccc(-n2ccc3ncc(-c4cccc(Cl)c4)nc32)cc1C1CCCC1]

The title compound was prepared in 4% overall yield as a light yellow solid according to the preparation of Example 1 using 2-(3-chlorophenyl)-4,4,5,5-tetramethyl-1,3,2-dioxaborolane in STEP A. [M+H] Calc'd for C₂₄H₂₀ClN₃O₂, 418. Found 418. ¹H NMR (400 MHz, DMSO-d₆) δ 9.25 (s, 1H), 8.51 (d, J=3.9 Hz, 1H), 8.28 (s, 2H), 8.23-8.17 (m, 1H), 7.81 (s, 1H), 7.70 (s, 1H), 7.61-7.50 (m, 2H), 7.00 (d, J=3.9 Hz, 1H), 4.06-3.91 (m, 1H), 2.17-2.09 (m, 2H), 1.90-1.79 (m, 2H), 1.78-1.63 (m, 4H).

### Example 4: 2-Cyclopentyl-4-(3-(m-tolyl)-5H-pyrrolo[2,3-b]pyrazin-5-yl)benzoic Acid

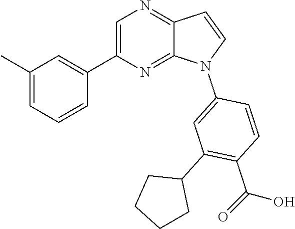 [SMILES: Cc1cccc(-c2cnc3ccn(-c4ccc(C(=O)O)c(C5CCCC5)c4)c3n2)c1]

The title compound was prepared in 12.3% overall yield as an off-white solid according to the preparation of Example 1 using 2-(3-chlorophenyl)-4,4,5,5-tetramethyl-1,3,2-dioxaborolane in STEP A. [M+H] Calc'd for C₂₅H₂₃N₃O₂, 398. Found 398. ¹H NMR (400 MHz, DMSO-d₆) δ 9.19 (s, 1H), 8.50 (d, J=3.9 Hz, 1H), 8.41-8.36 (m, 1H), 8.07 (s, 1H), 8.02 (d, J=7.8 Hz, 1H), 7.88 (d, J=8.4 Hz, 1H), 7.80-7.73 (m, 1H), 7.42 (t, J=7.7 Hz, 1H), 7.30 (d, J=7.6 Hz, 1H), 6.99 (d, J=3.8 Hz, 1H), 3.99-3.89 (m, 1H), 2.42 (s, 3H), 2.17-2.09 (m, 2H), 1.88-1.80 (m, 2H), 1.70-1.64 (m, 4H).

### Example 5: 2-(Ethylamino)-4-(3-phenylpyrrolo[2,3-b]pyrazin-5-yl)benzoic Acid

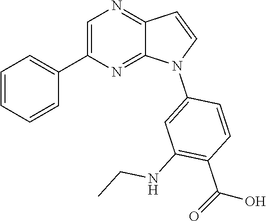 [SMILES: CCNc1cc(-n2ccc3ncc(-c4ccccc4)nc32)ccc1C(=O)O]

Step A. To a stirred solution of 3-chloro-5H-pyrrolo[2,3-b]pyrazine (3 g, 19.5 mmol) in DMF (30 mL) and water (6 mL) was added phenylboronic acid (3.57 g, 29.3 mmol), Pd(dppf)Cl₂ (300 mg, 0.41 mmol) and Cs₂CO₃ (14.1 g, 39.1 mmol). The resulting mixture purged with N₂ and allowed to stir at 100° C. for 3 h. Upon completion, the solvent was concentrated in vacuo and the residue taken up in water and extracted with ethyl acetate (3×). The combined organic layers were successively washed with brine, dried over anhydrous sodium sulfate, filtered, and concentrated in vacuo. The residue was purified by a silica gel column chromatography (PE/A=4:1 as eluent) to obtain 3-phenyl-5H-pyrrolo[2,3-b]pyrazine as a yellow solid (3.6 g, 94% yield). [M+H] Calc'd for C₁₂H₉N₃, 196. Found 196.

STEP B. To a stirred solution of 3-phenyl-5H-pyrrolo[2,3-b]pyrazine (400 mg, 2.1 mmol) in DMF (15 mL) was added methyl 2-fluoro-4-iodo-benzoate (689 mg, 2.46 mmol), CuI (80 mg, 0.41 mmol), N,N-dimethylcyclohexane-1,2-diamine (120 mg, 0.82 mmol) and K₃PO₄ (870 mg, 4.1 mmol). The resulting mixture was purged with N₂ and irradiated in the microwave at 110° C. for 2 h. Upon completion, the reaction was filtered and concentrated in vacuo. The residue was taken up in water and extracted with EA. The organic layers were successively dried over anhydrous sodium sulfate, filtered, and concentrated in vacuo. The residue purified by silica gel column chromatography (PE/EA=6:1 as eluent) to give methyl 2-fluoro-4-(3-phenylpyrrolo[2,3-b]pyrazin-5-yl)benzoate (400 mg, 45% yield) as a yellow solid. [M+H] Calc'd for C₂₀H₁₄FN₃O₂, 348. Found 348.

STEP C. To a stirred solution of 2-fluoro-4-(3-phenylpyrrolo[2,3-b]pyrazin-5-yl)benzoate (350 mg, 1.01 mmol) in DMA (5 mL) was added ethylamine (90 mg, 2.02 mmol) and DIEA (130 mg, 1.01 mmol). The reaction was stirred at 100° C. for 2 h. Upon completion, the reaction was filtered and concentrated in vacuo. The residue was taken up in water and extracted with ethyl acetate (3×). The organic layers were successively dried over anhydrous sodium sulfate, filtered, and concentrated in vacuo. The residue was purified by silica gel column chromatography (PE/EA=5:1 as eluent) to give methyl 2-(ethylamino)-4-(3-phenylpyrrolo[2,3-b]pyrazin-5-yl)benzoate (110 mg, 26% yield) as a yellow solid. [M+H] Calc'd for C₂₀H₁₈N₄O₂, 373. Found 373.

STEP D. To a stirred solution of methyl 2-(ethylamino)-4-(3-phenylpyrrolo[2,3-b]pyrazin-5-yl)benzoate (100 mg, 0.27 mmol) in THF (2 mL) and methanol (2 mL) was added NaOH (54 mg, 1.34 mmol) in water (1 mL). The reaction was stirred at 50° C. for 2 h. Upon completion the solvent were partially removed in vacuo and the pH was adjusted (˜4) with sat. citric acid. The precipitate were collected and dissolved in DMSO and purified by Prep-HPLC to afford 2-(ethylamino)-4-(3-phenylpyrrolo[2,3-b]pyrazin-5-yl)benzoic acid (48.4 mg, 49.4%) as a light yellow solid. [M+H] Calc'd for C₂₁H₁₈N₄O₂, 359. Found 359. ¹H NMR (300 MHz, DMSO-d₆) δ 9.18 (s, 1H), 8.51 (d, 1H), 8.28-8.20 (m, 2H), 7.99 (d, 1H), 7.68 (s, 1H), 7.57-7.45 (m, 3H), 7.20 (m, 1H), 6.97 (d, 1H), 3.39 (q, 2H), 1.32 (t, 3H). Prep-HPLC conditions [Column: XBridge Shield RP18 OBD Column 19*250 mm, 10 um; Mobile Phase A: Water (10 mmol/L NH₄HCO₃), Mobile Phase B: ACN; Flow rate: 25 mL/min; Gradient: 40% B to 50% B in 8 min; 254/210 nm; Rt: 7.5 min/]

### Example 6: 2-((Cyclopropylmethyl)amino)-4-(3-phenyl-5H-pyrrolo[2,3-b]pyrazin-5-yl)benzoic Acid

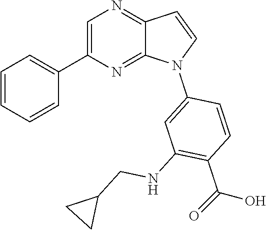 [SMILES: O=C(O)c1ccc(-n2ccc3ncc(-c4ccccc4)nc32)cc1NCC1CC1]

The title compound was prepared in 7% overall yield as a white solid according to the preparation of Example 5 using cyclopropylmethanamine in STEP C. [M+H] Calc'd for C₂₃H₂₀N₄O₂, 385. Found 385. S ¹H NMR (300 MHz, DMSO-d₆) δ 9.20 (s, 1H), 8.53 (d, J=3.9 Hz, 1H), 8.22 (d, J=7.2 Hz, 2H), 7.99 (d, J=8.6 Hz, 1H), 7.77 (s, 1H), 7.54 (d, J=7.6 Hz, 1H), 7.00 (d, J=4.1 Hz, 1H), 3.23 (d, J=6.8 Hz, 2H), 1.40 (s, 3H), 0.54 (s, 2H), 0.31 (d, J=5.2 Hz, 2H).

### Example 7: 2-(Cyclopentylamino)-4-(3-phenyl-5H-pyrrolo[2,3-b]pyrazin-5-yl)benzoic Acid

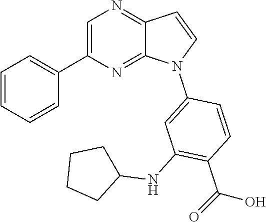 [SMILES: O=C(O)c1ccc(-n2ccc3ncc(-c4ccccc4)nc32)cc1NC1CCCC1]

The title compound was prepared in 17% overall yield as a white solid according to the preparation of Example 5 using cyclopentylamine in STEP C. [M+H] Calc'd for C₂₄H₂₂N₄O₂, 399. Found 399. ¹H NMR (300 MHz, DMSO-d₆) δ 9.18 (s, 1H), 8.49 (d, J=4.0 Hz, 1H), 8.22 (d, J=7.4 Hz, 2H), 7.98 (d, J=8.3 Hz, 1H), 7.72 (s, 1H), 7.60-7.46 (m, 3H), 7.12 (d, J=8.1 Hz, 1H), 6.99 (d, J=3.8 Hz, 1H), 4.03 (m, 1H), 2.10 (m, 2H), 1.73 (m, 2H), 1.58 (m, 4H).

### Example 8: 2-((Cyclopropylmethyl)amino)-4-(3-(6-fluoro-1-methyl-1H-indazol-5-yl)-5H-pyrrolo[2,3-b]pyrazin-5-yl)benzoic Acid

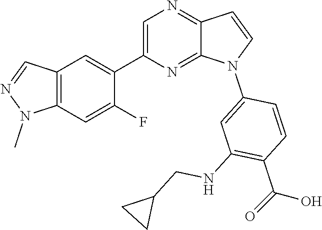 [SMILES: Cn1ncc2cc(-c3cnc4ccn(-c5ccc(C(=O)O)c(NCC6CC6)c5)c4n3)c(F)cc21]

The title compound was prepared in 17.5% overall yield as a yellow solid according to the preparation of Example 5 using 6-fluoro-1-methyl-5-(4,4,5,5-tetramethyl-1,3,2-dioxaborolan-2-yl)-1H-indazole in STEP A and cyclopropylmethanamine in STEP C. [M+H] Calc'd for C₂₅H₂₁FN₆O₂, 457. Found 457. ¹H NMR (DMSO-d₆, 400 MHz) δ 8.94 (d, 1H), 8.52 (d, 1H), 8.33 (d, 1H), 8.17 (s, 1H), 7.96 (d, 1H), 7.80-7.70 (m, 2H), 7.16 (d, 1H), 7.00 (d, 1H), 4.08 (s, 3H), 3.17 (d, 2H), 1.29-1.10 (m, 1H), 0.45-0.36 (m, 2H), 0.25-0.16 (m, 2H)

### Example 9: 4-(3-(2,4-Difluorophenyl)-5H-pyrrolo[2,3-b]pyrazin-5-yl)-2-(ethylamino)benzoic Acid

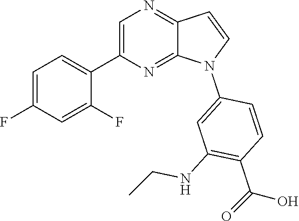 [SMILES: CCNc1cc(-n2ccc3ncc(-c4ccc(F)cc4F)nc32)ccc1C(=O)O]

The title compound was prepared in 4.2% overall yield as an off-white solid according to the preparation of Example 5 using (2,4-difluorophenyl)boronic acid in STEP A. [M+H] Calc'd for C₂₁H₁₆F₂N₄O₂, 395. Found 395. ¹H NMR (400 MHz, DMSO-d₆) δ 8.93 (d, J=2.5 Hz, 1H), 8.57 (d, J=3.9 Hz, 1H), 8.08 (td, J=8.9, 6.6 Hz, 1H), 7.96 (d, J=8.7 Hz, 1H), 7.62 (d, J=2.1 Hz, 1H), 7.48 (ddd, J=11.6, 9.2, 2.6 Hz, 1H), 7.32 (td, J=8.4, 2.6 Hz, 1H), 7.16 (dd, J=8.7, 2.1 Hz, 1H), 7.02 (d, J=3.9 Hz, 1H), 3.34 (d, J=7.1 Hz, 2H), 1.27 (t, J=7.1 Hz, 3H).

### Example 10: 2-(Cyclopropylamino)-4-(3-(2,4-difluorophenyl)-5H-pyrrolo[2,3-b]pyrazin-5-yl)benzoic Acid

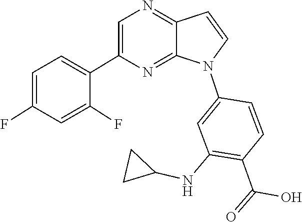 [SMILES: O=C(O)c1ccc(-n2ccc3ncc(-c4ccc(F)cc4F)nc32)cc1NC1CC1]

The title compound was prepared in 16.6% overall yield as an off-white solid according to the preparation of Example 5 using (2,4-difluorophenyl)boronic acid in STEP A and cyclopropanamine in STEP C. [M+H] Calc'd for C₂₁H₁₆F₂N₄O₂, 407. Found 407. ¹H NMR (300 MHz, DMSO-d₆) δ 12.81 (s, 1H), 8.92 (d, J=2.6 Hz, 1H), 8.52 (d, J=3.9 Hz, 1H), 8.16 (s, 1H), 8.07 (td, J=8.9, 6.7 Hz, 1H), 7.98-7.89 (m, 2H), 7.45 (ddd, J=11.7, 9.3, 2.6 Hz, 1H), 7.34-7.21 (m, 1H), 7.15 (dd, J=8.7, 2.2 Hz, 1H), 7.01 (d, J=3.9 Hz, 1H), 2.56 (dt, J=6.6, 3.2 Hz, 1H), 0.74 (td, J=6.7, 4.6 Hz, 2H), 0.57-0.46 (m, 2H).

### Example 11: 2-((Cyclopropylmethyl)amino)-4-(3-(4-fluorophenyl)-5H-pyrrolo[2,3-b]pyrazin-5-yl)benzoic Acid

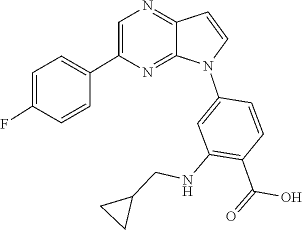 [SMILES: O=C(O)c1ccc(-n2ccc3ncc(-c4ccc(F)cc4)nc32)cc1NCC1CC1]

The title compound was prepared in 5% overall yield as a yellow solid according to the preparation of Example 5 using 4-fluoro-phenylboronic acid in STEP A and cyclopropylmethanamine in STEP C. [M+H] Calc'd for C₂₃H₁₉FN₄O₂, 403. Found 403. ¹H NMR (400 MHz, DMSO-d₆) δ 9.17 (s, 1H), 8.51 (d, J=3.9 Hz, 1H), 8.30-8.22 (m, 2H), 7.96-8.03 (d, J=8.7 Hz, 1H), 7.66 (d, J=2.2 Hz, 1H), 7.37 (dd, J=9.9, 7.7 Hz, 2H), 7.20 (dd, J=8.7, 2.1 Hz, 1H), 6.98 (d, J=3.9 Hz, 1H), 3.20 (d, J=6.9 Hz, 2H), 1.22 (q, J=5.5 Hz, 1H), 0.58-0.49 (m, 2H), 0.33-0.25 (m, 2H).

### Example 12: 2-(Cyclopropylmethylamino)-4-(3-(2-methyl-2H-indazol-5-yl)-5H-pyrrolo[2,3-b]pyrazin-5-yl)benzoic Acid

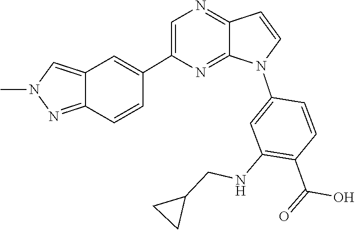 [SMILES: Cn1cc2cc(-c3cnc4ccn(-c5ccc(C(=O)O)c(NCC6CC6)c5)c4n3)ccc2n1]

The title compound was prepared in 36% overall yield as a yellow solid according to the preparation of Example 5 using (2-methyl-2H-indazol-5-yl)boronic acid in STEP A and cyclopropylmethanamine in STEP C. [M+H] Calc'd for C₂₅H₂₂N₆O₂, 439. Found 439. 1H NMR (300 MHz, DMSO-d₆) δ 9.22 (s, 1H), 8.58 (d, J=1.4 Hz, 1H), 8.51-8.42 (m, 2H), 8.30 (s, 1H), 8.13 (dd, J=9.1, 1.7 Hz, 1H), 8.00 (d, J=8.6 Hz, 1H), 7.76-7.67 (m, 2H), 7.17 (dd, J=8.7, 2.1 Hz, 1H), 6.96 (d, J=3.8 Hz, 1H), 4.22 (s, 3H), 3.23 (d, J=6.9 Hz, 2H), 1.25 (td, J=7.4, 3.6 Hz, OH), 0.59-0.45 (m, 2H), 0.35-0.24 (m, 2H).

### Example 13: 2-(Ethylamino)-4-(3-(2-methyl-2H-indazol-5-yl)-5H-pyrrolo[2,3-b]pyrazin-5-yl)benzoic Acid

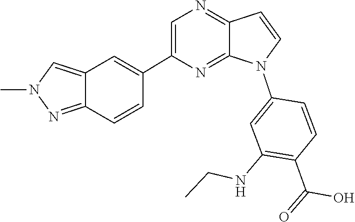 [SMILES: CCNc1cc(-n2ccc3ncc(-c4ccc5nn(C)cc5c4)nc32)ccc1C(=O)O]

The title compound was prepared in 39% overall yield as a yellow solid according to the preparation of Example 5 using (2-methyl-2H-indazol-5-yl)boronic acid in STEP A. [M+H] Calc'd for C₂₃H₂₀N₆O₂, 413. Found 413. ¹H NMR (300 MHz, DMSO-d₆) δ 9.22 (s, 1H), 8.58 (d, J=1.6 Hz, 1H), 8.52-8.42 (m, 2H), 8.14 (dd, J=9.1, 1.7 Hz, 1H), 8.00 (d, J=8.6 Hz, 1H), 7.78-7.67 (m, 2H), 7.16 (dd, J=8.7, 2.1 Hz, 1H), 6.96 (d, J=3.9 Hz, 1H), 4.22 (s, 3H), 3.39 (q, J=7.1 Hz, 2H), 1.33 (t, J=7.1 Hz, 3H).

### Example 14: 2-(Ethylamino)-4-(3-(1-methyl-1H-indazol-5-yl)-5H-pyrrolo[2,3-b]pyrazin-5-yl)benzoic Acid

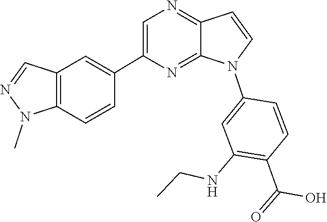 [SMILES: CCNc1cc(-n2ccc3ncc(-c4ccc5c(cnn5C)c4)nc32)ccc1C(=O)O]

The title compound was prepared in 9% overall yield as a light yellow solid according to the preparation of Example 5 using (1-methyl-1H-indazol-5-yl)boronic acid in STEP A. [M+H] Calc'd for C₂₃H₂₀N₆O₂, 413. Found 413. ¹H NMR (400 MHz, DMSO-d₆) δ 9.23 (s, 1H), 8.62 (s, 1H), 8.44 (d, J=3.9 Hz, 1H), 8.29 (dd, J=8.8, 1.7 Hz, 1H), 8.16 (s, 1H), 8.02 (d, J=8.4 Hz, 1H), 7.80 (d, J=8.9 Hz, 1H), 7.63 (s, 1H), 7.12 (dd, J=8.6, 2.1 Hz, 1H), 6.95 (d, J=3.9 Hz, 1H), 4.10 (s, 3H), 3.37 (q, J=7.1 Hz, 2H), 1.33 (t, J=7.1 Hz, 3H).

### Example 15: 2-(Cyclopropylamino)-4-(3-(4-fluorophenyl)-5H-pyrrolo[2,3-b]pyrazin-5-yl)benzoic Acid

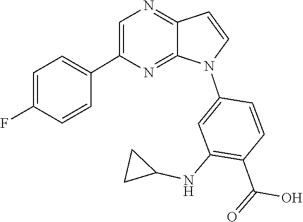 [SMILES: O=C(O)c1ccc(-n2ccc3ncc(-c4ccc(F)cc4)nc32)cc1NC1CC1]

The title compound was prepared in 4% overall yield as a white solid according to the preparation of Example 5 using (4-fluoro-phenylboronic) acid in STEP A and cyclopropaneamine in STEP C. [M+H] Calc'd for C₂₂H₁₇FN₄O₂, 389. Found 389. ¹H NMR (400 MHz, DMSO-d₆) δ 9.19 (s, 1H), 8.47 (d, J=4.0 Hz, 1H), 8.34-8.24 (m, 3H), 7.98 (d, J=8.7 Hz, 2H), 7.37 (t, J=8.8 Hz, 2H), 7.19 (dd, J=8.6, 2.2 Hz, 1H), 6.99 (d, J=3.8 Hz, 1H), 2.62 (tt, J=6.9, 3.6 Hz, 1H), 0.80 (h, J=4.6 Hz, 2H), 0.62-0.54 (m, 2H).

### Example 16: 2-(Ethylamino)-4-(3-(2-methyl-2H-indazol-6-yl)-5H-pyrrolo[2,3-b]pyrazin-5-yl)benzoic Acid

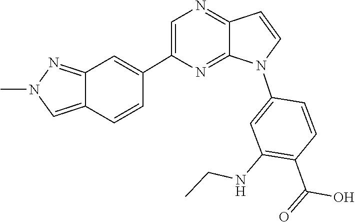 [SMILES: CCNc1cc(-n2ccc3ncc(-c4ccc5cn(C)nc5c4)nc32)ccc1C(=O)O]

The title compound was prepared in 10% overall yield as a light yellow solid according to the preparation of Example 5 using (2-methyl-2H-indazol-6-yl)boronic acid in STEP A. [M+H] Calc'd for C₂₃H₂₀N₆O₂, 413. Found 413. ¹H NMR (400 MHz, DMSO-d₆) δ 9.27 (s, 1H), 8.54-8.45 (m, 2H), 8.40 (s, 1H), 7.99 (d, J=8.7 Hz, 1H), 7.94 (dd, J=8.8, 1.5 Hz, 1H), 7.85 (dd, J=8.7, 0.9 Hz, 1H), 7.80 (d, J=2.1 Hz, 1H), 7.19 (dd, J=8.7, 2.1 Hz, 1H), 6.99 (d, J=3.9 Hz, 1H), 4.22 (s, 3H), 3.42 (q, J=7.1 Hz, 2H), 1.34 (t, J=7.1 Hz, 3H).

### Example 17: 2-((Cyclopropylmethyl)amino)-4-(3-(2-methyl-2H-indazol-6-yl)-5H-pyrrolo[2,3-b]pyrazin-5-yl)benzoic Acid

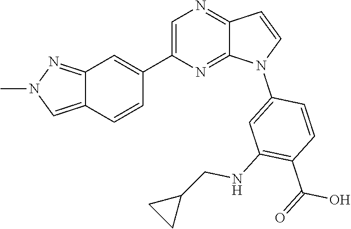 [SMILES: Cn1cc2ccc(-c3cnc4ccn(-c5ccc(C(=O)O)c(NCC6CC6)c5)c4n3)cc2n1]

The title compound was prepared in 8% overall yield as a light yellow solid according to the preparation of Example 5 using 6-fluoro-1-methyl-5-(4,4,5,5-tetramethyl-1,3,2-dioxaborolan-2-yl)-1H-indazole in STEP A and cyclopropylmethanamine in STEP C. [M+H] Calc'd for C₂₅H₂₂N₆O₂, 439. Found 439. ¹H NMR (400 MHz, DMSO-d₆) δ 9.27 (s, 1H), 8.52-8.43 (m, 2H), 8.40 (s, 1H), 8.00 (d, J=8.6 Hz, 1H), 7.92 (dd, J=8.8, 1.5 Hz, 1H), 7.83 (d, J=8.8 Hz, 1H), 7.76 (d, J=2.1 Hz, 1H), 7.18 (dd, J=8.6, 2.1 Hz, 1H), 6.98 (d, J=3.9 Hz, 1H), 4.22 (s, 3H), 3.24 (d, J=6.8 Hz, 2H), 1.31-1.21 (m, 1H), 0.58-0.46 (m, 2H), 0.39-0.29 (m, 2H).

### Example 18: 2-Cyclopentyl-4-(3-cyclopropyl-5H-pyrrolo[2,3-b]pyrazin-5-yl)benzoic Acid

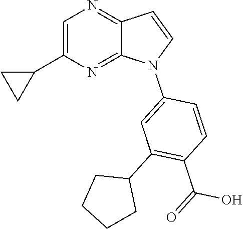 [SMILES: O=C(O)c1ccc(-n2ccc3ncc(C4CC4)nc32)cc1C1CCCC1]

STEP A. To a stirred solution of 3-chloro-5H-pyrrolo[2,3-b]pyrazine (5.00 g, 32.6 mmol) in MeCN (100 mL) was added (Boc)₂O (10.5 g, 48.8 mmol) and DMAP (795 mg, 6.51 mmol), and the mixture was allowed to stir at r.t. for 2 h. Upon completion the reaction was concentrated, and the residue was passed through a pad of silica gel to obtain tert-butyl 3-chloropyrrolo[2,3-b]pyrazine-5-carboxylate (7.4 g, 89.6%) as an off-white solid. [M+H] Calc.'d for C₁₁H₁₂ClN₃O₂, 254. Found 254.

STEP B. To a solution of tert-butyl 3-chloropyrrolo[2,3-b]pyrazine-5-carboxylate (7.0 g, 27.6 mmol) in toluene (10 mL) and water (1 mL) were added RuPhos Pd G3 (4.6 g, 5.5 mmol) and Cs₂CO₃ (27 g, 82.8 mmol). The reaction was purged with N₂ and stirred at 120° C. for 1 h. Upon completion the reaction mixture was filtered and concentrated in vacuo. The residue was passed through a pad of silica gel to obtain tert-butyl 3-cyclopropylpyrrolo[2,3-b]pyrazine-5-carboxylate (6.2 g, 87%) as an off-white solid. [M+H] Calc.'d for C₁₄H₁₇N₃O₂, 260. Found 260.

STEP C. To a stirred solution of tert-butyl 3-cyclopropylpyrrolo[2,3-b]pyrazine-5-carboxylate (6.50 g, 25.1 mmol) in DCM (20 mL) was added TFA (20 mL, 261 mmol) at 0° C. The reaction was warmed to r.t. and stirred for 2 h. Upon completion the reaction was concentrated in vacuo. The residue was purified by flash column chromatography to obtain 3-cyclopropyl-5H-pyrrolo[2,3-b]pyrazine (3.2 g, 80%) as a white solid. [M+H] Calc.'d for C₉H₉N₃, 160. Found 160.

STEP D. To a solution of 3-cyclopropyl-5H-pyrrolo[2,3-b]pyrazine (500 mg, 3.14 mmol) in 1,4-dioxane (30 mL) was added methyl 4-bromo-2-cyclopentyl-benzoate (1.07 mg, 3.77 mmol), CuI (119 mg, 0.63 mmol), dimethylcyclohexane-1,2-diamine (178 mg, 1.26 mmol) and K₃PO₄ (1.33 mg, 6.3 mmol). The reaction mixture was purged with N₂ and stirred at 110° C. for 2 h. Upon completion the reaction was filtered and concentrated in vacuo. The residue was passed through a pad of silica gel to obtain methyl 2-cyclopentyl-4-(3-cyclopropylpyrrolo[2,3-b]pyrazin-5-yl)benzoate (630 mg, 56%) as a light semi-solid. [M+H] Calc.'d for C₂₂H₂₃N₃O₂, 362. Found 362.

STEP E. To a stirred solution of methyl 2-cyclopentyl-4-(3-cyclopropylpyrrolo[2,3-b]pyrazin-5-yl)benzoate (120 mg, 0.33 mmol) in methanol (1.5 mL), THF (1.5 mL) and water (0.6 mL) was added NaOH (120 mg, 3 mmol). The reaction was stirred at 50° C. for 2 h. Upon completion the reaction mixture was concentrated in vacuo. The residue was taken up in water and pH adjusted to 3-4, filtered. The precipitate was collected and further purified by Prep HPLC to afford the title compound (38.2 mg, 33% yield) as a white solid. [M+H] Calc.'d for C₂₁H₂₁N₃O₂, 348. Found 348. ¹H NMR (400 MHz, DMSO-d₆) δ 8.55 (s, 1H), 8.26 (d, J=3.9 Hz, 1H), 8.11 (d, J=2.2 Hz, 1H), 7.75 (d, J=8.3 Hz, 1H), 7.57 (m, J=8.4, 2.3 Hz, 1H), 6.84 (d, J=3.9 Hz, 1H), 4.01 (d, J=12.6 Hz, 1H), 2.31 (m, J=8.1, 4.1 Hz, 1H), 2.08 (s, 2H), 1.81 (s, 2H), 1.63 (q, J=7.3 Hz, 4H), 1.11-0.96 (m, 4H). [Column: XBridge Shield RP18 OBD Column 19*250 mm, 10 um; Mobile Phase A: Water (10 mmol/L NH₄HCO₃), Mobile Phase B: ACN; Flow rate: 25 mL/min; Gradient: 5% B to 60% B in 7.5 min; 254/210 nm; Rt: 6.2 min]

### Example 19: N-Cyano-2-cyclopentyl-4-(3-cyclopropyl-5H-pyrrolo[2,3-b]pyrazin-5-yl)benzamide

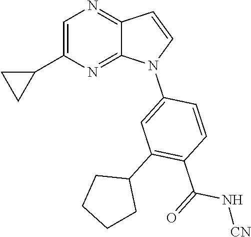 [SMILES: N#CNC(=O)c1ccc(-n2ccc3ncc(C4CC4)nc32)cc1C1CCCC1]

To a stirred solution of 2-cyclopentyl-4-(3-cyclopropylpyrrolo[2,3-b]pyrazin-5-yl)benzoic acid (70 mg, 0.20 mmol) in DCM (2 mL) was added DCC (62 mg, 0.30 mmol), DMAP (37 mg, 0.30 mmol) and cyanamide (25 mg, 0.60 mmol) at 0° C. The reaction was allowed to at 45° C. for 3 h. Water was added to the reaction and extracted with DCM (3×). The organic layers were dried over sodium sulfate and concentrated in vacuo. The residue was passed through a pad of silica gel and further purified by Prep-HPLC to furnish the title compound (32.4 mg, 43% yield) as a yellow solid. [M+H] Calc.'d for C₂₂H₂₁N₅O, 372. Found 372. ¹H NMR (400 MHz, DMSO-d₆) δ 12.30 (s, 1H), 8.58 (s, 1H), 8.35 (d, J=3.9 Hz, 1H), 8.29 (d, J=2.2 Hz, 1H), 7.79 (m, J=8.4, 2.2 Hz, 1H), 7.69 (d, J=8.4 Hz, 1H), 6.89 (d, J=3.9 Hz, 1H), 3.42 (t, J=8.3 Hz, 1H), 2.34 (m, J=8.0, 4.7 Hz, 1H), 2.09 (m, J=10.9, 7.6, 3.9 Hz, 2H), 1.87-1.81 (m, 2H), 1.69 (s, 4H), 1.09 (m, J=8.0, 5.7, 2.9 Hz, 2H), 1.01 (m, J=4.5, 2.6 Hz, 2H). [Column: Sunfire Prep C18 OBD Column, 10 um, 19*250 mm; Mobile Phase A: Water (0.05% TFA), Mobile Phase B: ACN; Flow rate: 25 mL/min; Gradient: 20% B to 60% B in 8 min; 254/210 nm; Rt: 7.65 min]

### Example 20: 5-(3-cyclopentyl-4-(2H-tetrazol-5-yl)phenyl)-3-cyclopropyl-5H-pyrrolo[2,3-b]pyrazine

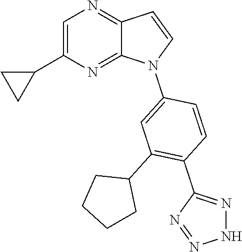 [SMILES: c1cc(-c2nn[nH]n2)c(C2CCCC2)cc1-n1ccc2ncc(C3CC3)nc21]

STEP A. To a solution of 2-cyclopentyl-4-(3-cyclopropylpyrrolo[2,3-b]pyrazin-5-yl)benzoic acid (250 mg, 0.72 mmol), NH₄Cl (114 mg, 2.11 mmol) and HATU (410 mg, 1.08 mmol) in DMF (5 mL) was added DIEA (0.61 mL, 3.48 mmol) dropwise at 0° C. The resulting solution was stirred at r.t. for 2 h. Upon completion water was added, and the mixture was extracted with DCM (3×). The organic layers were washed with brine, dried over anhydrous sodium sulfate, filtered, and concentrated in vacuo. The residue was purified by Prep-TLC (DCM/MeOH, 10:1) to afford 2-cyclopentyl-4-(3-cyclopropylpyrrolo[2,3-b]pyrazin-5-yl)benzamide (220 mg, 88% yield) as an off-white solid. [M+H] Calc.'d for C₂₁H₂₂N₄O, 347. Found 347.

STEP B. A solution of 2-cyclopentyl-4-(3-cyclopropylpyrrolo[2,3-b]pyrazin-5-yl)benzamide (269 mg, 0.78 mmol) in POCl₃ (10 mL, 107 mmol) was stirred at 65° C. for 2 h. The resulting solution was concentrated in vacuo. The residue was taken up in water, and the pH adjusted to 8-9 with saturated Na₂CO₃. The resulting mixture was extracted with DCM (3×). The combined organic layers were dried over anhydrous sodium sulfate, filtered, and concentrated in vacuo. The residue was purified by Prep-TLC (PE/EA=1:1) to obtain 2-cyclopentyl-4-(3-cyclopropylpyrrolo[2,3-b]pyrazin-5-yl)benzonitrile (200 mg, 78% yield) as an off-white solid. [M+H] Calc.'d for C₂₁H₂₀N₄, 329. Found 329.

STEP C. A 20 mL sealed tube charged with 2-cyclopentyl-4-(3-cyclopropylpyrrolo[2,3-b]pyrazin-5-yl)benzonitrile (180 mg, 0.55 mmol), NaN₃ (160 mg, 2.46 mmol) and TEA·HCl (300 mg, 2.17 mmol) in NMP (4 mL) was stirred at 150° C. for 8 h. The resulting mixture was suspended in brine and extracted with THF (3×). The organic layers were dried over anhydrous sodium sulfate, filtered, and concentrated in vacuo. The residue was purified by Prep-HPLC and pure fractions were concentrated in vacuo to furnish the title compound (49.9 mg, 24.1% yield) as a light-yellow solid. [M+H] Calc.'d for C₂₁H₂₁N₇, 372. Found 372. ¹H NMR (400 MHz, DMSO-d₆) δ 8.59 (s, 1H), 8.37 (m, 2H), 7.84 (m, 1H), 7.73 (d, 1H), 6.90 (d, 1H), 3.54-3.48 (m, 1H), 2.39-2.32 (m, 1H), 2.08-2.01 (m, 2H), 1.87-1.78 (m, 2H), 1.71-1.62 (m, 4H), 1.15-1.01 (m, 4H). Prep-HPLC [Column: Sunfire Prep C18 OBD Column, 10 um, 19*250 mm; Mobile Phase A: Water (0.05% TFA), Mobile Phase B: ACN; Flow rate: 25 mL/min; Gradient: 20% B to 50% B in 7 min; 254/210 nm; Rt: 6.95 min].

### Example 21: 2-Cyclopentyl-4-(3-cyclopropyl-5H-pyrrolo[2,3-b]pyrazin-5-yl)-N-((trifluoromethyl)sulfonyl)benzamide

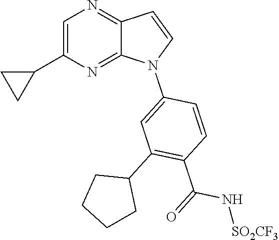

To a stirred solution of 2-cyclopentyl-4-(3-cyclopropylpyrrolo[2,3-b]pyrazin-5-yl)benzoic acid (60 mg, 0.17 mmol) in DCM (2 mL) was added DCC (53 mg, 0.26 mmol) at r.t., and the reaction was stirred for 30 min. DMAP (32 mg, 0.26 mmol) and trifluoromethanesulfonamide (77.2 mg, 0.52 mmol) were added at 0° C., and the mixture was stirred at 45° C. for 3 h. Water was added, and the content extracted with DCM (3×). The combined organic layers were dried over sodium sulfate and concentrated in vacuo. The crude product was passed through a pad of silica gel and further purified by Prep-HPLC to afford the title compound (32.5 mg, 39% yield) as a white solid. [M+H] Calc.'d for C₂₂H₂₁F₃N₄O₃S, 479. Found 479. ¹H NMR (300 MHz, MeOD) δ 8.43 (s, 1H), 8.07 (m, J=8.2, 3.1 Hz, 2H), 7.65 (d, J=8.4 Hz, 1H), 7.49 (m, J=8.3, 2.2 Hz, 1H), 6.78 (d, J=3.9 Hz, 1H), 3.88-3.77 (m, 1H), 2.37-2.25 (m, 1H), 2.22 (s, 2H), 1.89 (s, 2H), 1.74 (m, J=6.8, 3.8 Hz, 1H), 1.15-1.05 (m, 4H). Prep-HPLC conditions [Column: XBridge Prep C18 OBD Column, 5 um, 19*150 mm; Mobile Phase A: Water (10 mmoL/L NH₄HCO₃), Mobile Phase B: ACN; Flow rate: 25 mL/min; Gradient: 10% B to 70% B in 7 min; 254/210 nm; Rt: 6.95 min]

### Example 22: 2-Cyclopentyl-4-(3-phenyl-5H-pyrrolo[2,3-b]pyrazin-5-yl)benzonitrile

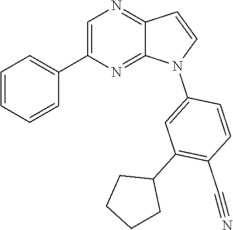 [SMILES: N#Cc1ccc(-n2ccc3ncc(-c4ccccc4)nc32)cc1C1CCCC1]

STEP A. A mixture containing 2-cyclopentyl-4-(3-phenylpyrrolo[2,3-b]pyrazin-5-yl)benzoic acid (280 mg, 0.73 mmol), HATU (595 mg, 1.56 mmol), NH₄Cl (127 mg, 2.35 mmol), and DIEA (0.68 mL, 3.91 mmol) in DMF (5 mL) was stirred under N₂ at r.t. for 3 h. Upon completion water (50 mL) was added, and the mixture was extracted with ethyl acetate (3×). The combined organic layers were successively washed with brine, dried over anhydrous sodium sulfate, and concentrated in vacuo. The crude product was purified by Prep-TLC (EA/PE=2:1) to obtain the target product (120 mg, 43% yield) as a yellow solid. [M+H] Calc.'d for C₂₄H₂₂N₄O, 383. Found 383.

STEP B. A mixture containing 2-cyclopentyl-4-(3-phenylpyrrolo[2,3-b]pyrazine-5-yl)benzamide (180 mg, 0.47 mmol) in POCl₃ (5 mL, 53.64 mmol) was stirred at 65° C. for 2 h. Upon completion the reaction was concentrated in vacuo. The residue was suspended in brine (3 mL) and the pH adjusted to ˜8 with sat. Na₂CO₃. To the mixture was added THF, and the organic layers were successively washed with brine, dried over sodium sulfate, filtered and concentrated in vacuo. The residue was purified by Prep-TLC to afford the title compound (27.5 mg, 16% yield) as an off white solid. [M+H] Calc.'d for C₂₄H₂₀N₄, 365. Found 365.

### Example 23: 5-(3-Cyclopentyl-4-(2H-tetrazol-5-yl)phenyl)-3-phenyl-5H-pyrrolo[2,3-b]pyrazine

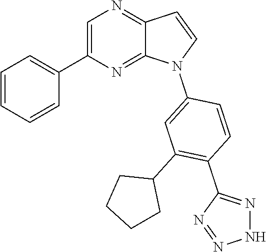 [SMILES: c1ccc(-c2cnc3ccn(-c4ccc(-c5nn[nH]n5)c(C5CCCC5)c4)c3n2)cc1]

To a stirred solution of 2-cyclopentyl-4-(3-phenylpyrrolo[2,3-b]pyrazin-5-yl)benzonitrile (65 mg, 0.18 mmol) in NMP (4 mL) was added N,N-diethylethanamine hydrochloride (123 mg, 0.89 mmol) and NaN₃ (58 mg, 0.89 mmol). The mixture was stirred at 150° C. for 5 h. Upon completion the reaction mixture was diluted with THF, and the combined organic layers were successively washed with brine, dried over sodium sulfate, filtered, and concentrated in vacuo. The residue was purified by Prep-TLC and then further purified by Prep-HPLC to afford the title compound (33.5 mg, 72.7% yield) as a light yellow solid. [M+H] Calc.'d for C₂₄H₂₁N₇, 408. Found 408. ¹H NMR (400 MHz, DMSO-d₆) δ 9.22 (s, 1H), 8.59 (d, J=3.9 Hz, 1H), 8.51 (d, J=2.3 Hz, 1H), 8.28-8.20 (m, 2H), 8.00 (dd, J=8.4, 2.3 Hz, 1H), 7.79 (d, J=8.4 Hz, 1H), 7.55 (dd, J=8.2, 6.4 Hz, 2H), 7.53-7.45 (m, 1H), 7.03 (d, J=3.8 Hz, 1H), 3.59-3.51 (m, 1H), 2.07 (m, J=13.6, 7.8, 5.2, 2.6 Hz, 2H), 1.91-1.61 (m, 4H). Prep-HPLC conditions [Column: Xselect CSH OBD Column 30*150 mm 5 um n; Mobile Phase A: Water (0.05% TFA), Mobile Phase B: ACN; Flow rate: 60 mL/min; Gradient: 80% B to 100% B in 8 min; 254/210 nm; Rt: 7.65 min].

### Example 24. 2-(azetidin-3-yloxy)-4-(3-(3,5-dimethylphenyl)-5H-pyrrolo[2,3-b]pyrazin-5-yl)benzoic Acid

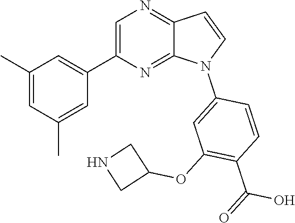 [SMILES: Cc1cc(C)cc(-c2cnc3ccn(-c4ccc(C(=O)O)c(OC5CNC5)c4)c3n2)c1]

STEP A. To a stirred solution of methyl 4-bromo-2-hydroxy-benzoate (2 g, 8.7 mmol) in THF (50 mL) was added tert-butyl 3-hydroxyazetidine-1-carboxylate (4.5 g, 26 mmol) and PPh₃ (6.8 g, 26 mmol) at room temperature under a nitrogen atmosphere. DIAD (7.0017 g, 34.626 mmol) was added dropwise to the mixture under N₂. The resulting mixture was stirred at 60° C. and stirred overnight. Upon completion the reaction mixture was diluted with water, extracted with ethyl acetate (3×), the combined organic layers were dried over sodium sulfate, and concentrated in vacuo. The crude product was purified by silica gel column chromatography to afford tert-butyl 3-(5-bromo-2-methoxycarbonyl-phenoxy)azetidine-1-carboxylate (3 g) as a pink oil. [M+H] Calc'd for C₁₅H₁₈BrNO₅, 372. Found, 372.

STEP B. To a 40 mL sealed tube was charged with 3-(3,5-dimethylphenyl)-5H-pyrrolo[2,3-b]pyrazine (200 mg, 0.9 mmol), tert-butyl 3-(5-bromo-2-methoxycarbonyl-phenoxy)azetidine-1-carboxylate (623 mg, 1.6 mmol), dimethylcyclohexane-1,2-diamine (51 mg, 0.36 mmol), CuI (34 mg, 0.18 mmol), and K₃PO₄ (380 mg, 1.8 mmol) in 1,4-dioxane (15 mL). The resulting mixture was bubbled with N₂ for 5 minutes. The reaction mixture was stirred at 100° C. overnight. Upon completion, the reaction mixture was diluted with water, extracted with ethyl acetate (3×), and the combined organic layers were successively washed with water, brine, dried over anhydrous sodium sulfate, filtered and concentrated in vacuo. The residue was purified by silica gel column chromatography to give tert-butyl 3-[5-[3-(3,5-dimethylphenyl)pyrrolo[2,3-b]pyrazin-5-yl]-2-methoxycarbonyl-phenoxy]azetidine-1-carboxylate (330 mg) as a yellow solid. [M+H] Calc'd for C₃₀H₃₂N₄O₅, 529. Found, 529.

STEP C. To a stirred solution of tert-butyl 3-[5-[3-(3,5-dimethylphenyl)pyrrolo[2,3-b]pyrazin-5-yl]-2-methoxycarbonyl-phenoxy]azetidine-1-carboxylate (330 mg, 0.62 mmol) in THF (10 mL) and methanol (10 mL) was added a solution of NaOH (125 mg, 3.1 mmol) in water (10 mL) at room temperature. The reaction was stirred at 50° C. for 3 h. Upon completion, the reaction mixture was concentrated and pH adjusted to ˜3-4 with citric acid. The resulting mixture was added brine and extracted with DCM. The combined organic layers were dried over sodium sulfate, concentrated in vacuo to give of 2-(1-tert-butoxycarbonylazetidin-3-yl)oxy-4-[3-(3,5-dimethylphenyl)pyrrolo[2,3-b]pyrazin-5-yl]benzoic acid (300 mg) as a yellow solid. [M+H] Calc'd for C₂₉H₃₀N₄O₅, 515. Found, 515.

STEP D. To a stirred solution of 2-(1-tert-butoxycarbonylazetidin-3-yl)oxy-4-[3-(3,5-dimethylphenyl)pyrrolo[2,3-b]pyrazin-5-yl]benzoic acid (290 mg, 0.56 mmol) in DCM (10 mL) was added TFA (10 mL) dropwise at 0° C. The resulting solution was stirred at room temperature for 4 h. Upon completion the mixture was concentrated in vacuo and the residue purified by Prep-HPLC to obtain title compound (46.9 mg, 20%) as a yellow solid. [M+H] Calc'd for C₂₄H₂₂N₄O₃, 415. Found, 415. ¹H NMR (400 MHz, Methanol-d4) δ 9.00 (s, 1H), 8.28 (d, J=3.9 Hz, 1H), 8.16 (d, J=8.5 Hz, 1H), 7.73 (d, J=6.0 Hz, 3H), 7.67 (d, J=2.0 Hz, 1H), 7.16 (s, 1H), 6.94 (d, J=3.9 Hz, 1H), 5.37-5.27 (m, 1H), 4.56 (dd, J=12.2, 6.4 Hz, 2H), 4.37 (dd, J=12.5, 4.4 Hz, 2H), 2.44 (s, 6H). Prep-HPLC conditions [Column: Xselect CSH OBD Column 30*150 mm 5 um n; Mobile Phase A: Water (0.05% TFA), Mobile Phase B: ACN; Flow rate: 25 mL/min; Gradient: 20% B to 50% B in 8 min; 254/210 nm; Rt: 7.65 min]

### Example 25. 2-(azetidin-3-ylamino)-4-(3-(3,5-dimethylphenyl)-5H-pyrrolo[2,3-b]pyrazin-5-yl)benzoic Acid

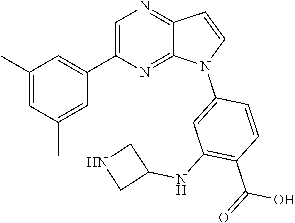 [SMILES: Cc1cc(C)cc(-c2cnc3ccn(-c4ccc(C(=O)O)c(NC5CNC5)c4)c3n2)c1]

STEP A. To a stirred solution of K₃PO₄ (1.14 g, 5.4 mmol) in DMF (15 mL) was added CuI (102 mg, 0.54 mmol), (1S,2S)—N1,N2-dimethylcyclohexane-1,2-diamine (153 mg, 1.1 mmol), 3-(3,5-dimethylphenyl)-5H-pyrrolo[2,3-b]pyrazine (600 mg, 2.7 mmol) and methyl 4-bromo-2-fluoro-benzoate (626 mg, 2.7 mmol) under N₂. The resulting mixture was stirred at 100° C. for 1 h. The reaction mixture was diluted with water and extracted with EA. The organic layers were successively dried over Na₂SO₄, filtered, and concentrated in vacuo. The residue was passed though silica gel to obtain methyl 4-[3-(3, 5-dimethylphenyl) pyrrolo[2,3-b]pyrazin-5-yl]-2-fluoro-benzoate (270 mg, 26.8% yield) as a light-yellow solid. MS: [M+H]=376.

STEP B. To a stirred solution of methyl 4-[3-(3, 5-dimethylphenyl) pyrrolo[2,3-b]pyrazin-5-yl]-2-fluoro-benzoate (60 mg, 0.16 mmol) in NMP (1 mL) were added DIEA (2 mL, 11.48 mmol) and tert-butyl 3-aminoazetidine-1-carboxylate (0.2 mL, 0.16 mmol). The reaction was stirred at 120° C. for 2 h. The reaction mixture was diluted with EA and washed with water. The reaction mixture was diluted with water and extracted with EA. The organic layers were successively dried over Na₂SO₄, filtered, and concentrated in vacuo. The obtained orange sticky solid was purified by Prep TLC to obtain tert-butyl 3-[5-[3-(3, 5-dimethylphenyl) pyrrolo[2,3-b]pyrazin-5-yl]-2-methoxycarbonyl-anilino]azetidine-1-carboxylate (70 mg, 84.328% yield) as a light-yellow solid. MS: [M+H]=528.

STEP C. To a stirred solution of tert-butyl 3-[5-[3-(3, 5-dimethylphenyl) pyrrolo[2,3-b]pyrazin-5-yl]-2-methoxycarbonyl-anilino]azetidine-1-carboxylate (100 mg, 0.19 mmol) in THF (2 mL) and MeOH (2 mL) was added a solution of NaOH (173 mg, 4.3 mmol) in Water (2 mL). The reaction was stirred at 50° C. for 1 h. The resulting mixture was concentrated to remove organic solvents and the residue was added 1 M HCl to adjust the pH˜4, and extracted with THF. The extracts were dried over Na₂SO₄, filtered and concentrated in vacuo. The residue was purified by Prep-TLC (DCM/MeOH, 10:1) to obtain 2-[(1-tert-butoxycarbonylazetidin-3-yl)amino]-4-[3-(3, 5-dimethylphenyl)pyrrolo[2,3-b]pyrazin-5-yl]benzoic acid (80 mg, 82% yield) as a light-yellow solid. MS: [M+H]=514.

### Example 26. (R) and (S)-4-(3-(3,5-dimethylphenyl)-5H-pyrrolo[2,3-b]pyrazin-5-yl)-2-(pyrrolidin-3-yl) benzoic Acid

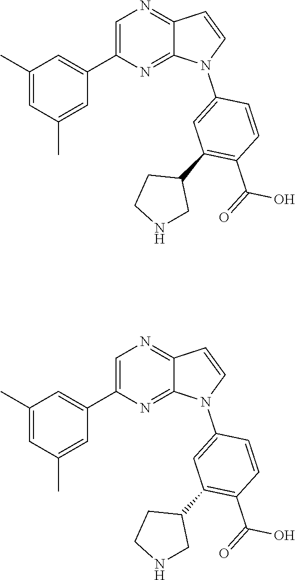

STEP A. To a stirred solution of tert-butyl 3-(4,4,5,5-tetramethyl-1,3,2-dioxaborolan-2-yl)-2,5-dihydropyrrole-1-carboxylate (500 mg, 1.7 mmol) in 1,4-Dioxane (10 mL) and Water (2 mL) were added methyl 4-bromo-2-iodo-benzoate (635.26 mg, 1.86 mmol) and Na₂CO₃ (538 mg, 5.1 mmol). Pd(dppf)Cl₂ (124 mg, 0.17 mmol) was added to the reaction mixture under nitrogen atmosphere. The reaction was warmed to 80° C. overnight. Upon completion, and the mixture was quenched with water, extracted with EA (3×). The organic layers were successively washed with brine, concentrated in vacuo and purified by flash column chromatography to afford tert-butyl 3-(5-bromo-2-methoxycarbonyl-phenyl)-2,5-dihydropyrrole-1-carboxylate (500 mg. 1.3 mmol) as a yellow oil. MS: [M+H]=384.

STEP B. To a stirred solution of tert-butyl 3-(5-bromo-2-methoxycarbonyl-phenyl)-2,5-dihydropyrrole-1-carboxylate (500 mg, 1.3 mmol) in Toluene (10 mL) was added PtO₂ (119 mg, 0.52 mmol) under a nitrogen atmosphere, the mixture was then vacuum and backfilled with H₂. The reaction was stirred at room temperature overnight. Upon completion, the mixture was filtered, concentrated in vacuo and purified by column chromatography (PE:EA=5:1) to afford tert-butyl 3-(5-bromo-2-methoxycarbonyl-phenyl)pyrrolidine-1-carboxylate (500 mg, 1.3 mmol) as an off-white oil. MS: [M+H]=386.

STEP C. To a stirred solution of 3-(3,5-dimethylphenyl)-5H-pyrrolo[2,3-b]pyrazine (300 mg, 1.34 mmol) in 1,4-Dioxane (8 mL) were added tert-butyl 3-(5-bromo-2-methoxycarbonyl-phenyl)pyrrolidine-1-carboxylate (516 mg, 1.3 mmol) and K₃PO₄ (855 mg, 4.0 mmol). CuI (51 mg, 0.27 mmol) and dimethylcyclohexane-1,2-diamine (76 mg, 0.54 mmol) were added to the reaction mixture under nitrogen atmosphere. The reaction was stirred at 100° C. overnight. Upon completion, the reaction mixture was concentrated in vacuo and purified by column chromatography to afford tert-butyl 3-[5-[3-(3,5-dimethylphenyl)pyrrolo[2,3-b]pyrazin-5-yl]-2-methoxycarbonyl-phenyl]pyrrolidine-1-carboxylate, as a yellow solid (522 mg). The solid was further purified by Prep-HPLC and separated by Anal-SFC. Optically pure fractions were combined and concentrated in vacuo to obtain Peak A (204 mg) and Peak B (169 mg) as solids. MS: [M+H]=527.

STEP D. To a stirred solution of tert-butyl 3-[5-[3-(3,5-dimethylphenyl)pyrrolo[2,3-b]-pyrazin-5-yl]-2-methoxycarbonyl-phenyl]pyrrolidine-1-carboxylate (peak A, 204 mg, 0.39 mmol) in DCM (15 mL) was added TFA (5 mL, 61 mmol). The reaction was stirred at room temperature for 2 h. LCMS showed the reaction completed, and the mixture was concentrated to afford crude product (165 mg, 100% yield). The crude product was taken up in a mixture of THF (5 mL), Methanol (5 mL) and Water (2.5 mL) and was added LiOH (60 mg, 2.5 mmol). The reaction was stirred at room temperature for 3 h. Upon completion, and the reaction mixture was concentrated in vacuo. The crude was purified by Prep-HPLC to obtain 4-[3-(3,5-dimethylphenyl)pyrrolo[2,3-b]pyrazin-5-yl]-2-pyrrolidin-3-yl-benzoic acid, as an off-white solid (16.4 mg, 10.2% yield). MS:[M+H] Calc'd for C₂₅H₂₄N₄O₂, 413. Found, 413. ¹H NMR (400 MHz, Methanol-d4) δ 8.81 (s, 1H), 8.29 (d, J=3.9 Hz, 1H), 8.16 (d, J=2.2 Hz, 1H), 8.11 (d, J=8.5 Hz, 1H), 7.86 (dd, J=8.3, 2.1 Hz, 1H), 7.55 (s, 2H), 7.09 (s, 1H), 6.91 (d, J=3.9 Hz, 1H), 4.52 (p, J=9.9, 9.2 Hz, 1H), 3.87 (dd, J=11.5, 8.0 Hz, 1H), 3.63 (ddd, J=11.6, 8.2, 3.2 Hz, 1H), 3.45 (td, J=11.3, 10.7, 7.1 Hz, 1H), 3.26-3.20 (m, 1H), 2.58 (d, J=10.0 Hz, 1H), 2.55 (s, 7H). Prep-HPLC conditions [Column: XBridge Prep OBD C18 Column 30×150 mm 5 um; Mobile Phase A: Water (10 MMOL/L NH₄HCO₃), Mobile Phase B: ACN; Flow rate: 60 mL/min; Gradient: 17% B to 34% B in 9 min; 254/210 nm; Rt: 9 min]

The title compound was prepared in 27% yield using tert-butyl 3-[5-[3-(3,5-dimethylphenyl)pyrrolo[2,3-b]pyrazin-5-yl]-2-methoxycarbonyl-phenyl]pyrrolidine-1-carboxylate (PEAK B) from STEP C. [M+H] Calc'd for C₂₅H₂₄N₄O₂, 413. Found, 413.

¹H NMR (400 MHz, Methanol-d4) δ 8.97 (s, 1H), 8.36 (d, J=2.2 Hz, 1H), 8.35 (d, J=3.9 Hz, 1H), 8.28 (d, J=8.5 Hz, 1H), 7.96 (dd, J=8.6, 2.2 Hz, 1H), 7.73 (s, 2H), 7.11 (s, 1H), 6.90 (d, J=3.9 Hz, 1H), 4.72-4.58 (m, 1H), 3.91 (dd, J=11.5, 8.0 Hz, 1H), 3.63 (ddd, J=12.0, 8.4, 3.6 Hz, 1H), 3.46 (td, J=11.1, 10.5, 7.3 Hz, 1H), 3.26 (d, J=10.7 Hz, 1H), 2.61-2.58 (m, 1H), 2.57 (d, J=9.6 Hz, 1H), 2.44 (s, 6H). Prep-HPLC conditions [Column: Select CSH OBD Column 30*150 mm 5 um n; Mobile Phase

A: Water (0.05% TFA), Mobile Phase B: ACN; Flow rate: 60 mL/min; Gradient: 20% B to 47% B in 8 min; 254/210 nm; Rt: 6.93 min]

### Example 27: 2-(azetidin-3-ylmethyl)-4-(3-(3,5-dimethylphenyl)-5H-pyrrolo[2,3-b]pyrazin-5-yl) benzoic Acid

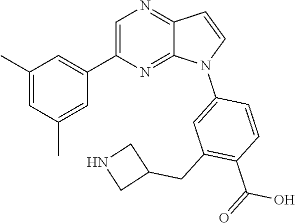 [SMILES: Cc1cc(C)cc(-c2cnc3ccn(-c4ccc(C(=O)O)c(CC5CNC5)c4)c3n2)c1]

STEP A. To a stirred mixture of Zn (414 mg, 6.3 mmol) in DMA (3 mL) was added a solution of BrCH2CH2Br (0.1 mL, 16.4 mmol) and TMSCl (0.2 mL, 16.4 mmol) in DMA (0.50 mL) at 40° C., and the resulting mixture was stirred for 30 min. A solution of tert-butyl 3-(iodomethyl) azetidine-1-carboxylate (900 mg, 3.03 mmol) in DMA (1.0 mL) was added to the reaction mixture and stirred at 40° C. for 30 min. The resulting mixture was transferred to a mixture of methyl 4-bromo-2-iodo-benzoate (500 mg, 1.47 mmol), Pd(dppf)Cl₂ (500 mg, 0.68 mmol) in THF (5 mL) with a syringe. The resulting solution was stirred at 60° C. for 2 h under N₂. Upon completion, the resulting mixture was concentrated in vacuo and the residue was purified by a silica gel column with PE/EA (20:1 to 10:1). Further Prep-HPLC purification to obtain tert-butyl 3-[(5-bromo-2-methoxycarbonyl-phenyl)methyl]azetidine-1-carboxylate (200 mg, 35.5% yield) as a light-yellow semi-solid. MS:[M+H]=384

STEP B. To a stirred mixture of (1R,2R)—N1,N2-dimethylcyclohexane-1,2-diamine (60 mg, 0.42 mmol) in 1,4-Dioxane (10 mL) was added K₃PO₄ (0.1 mL, 2.1 mmol), CuI (0.2 mL, 0.21 mmol), tert-butyl 3-[(5-bromo-2-methoxycarbonyl-phenyl)methyl]azetidine-1-carboxylate (270 mg, 0.70 mmol) and 3-(3,5-dimethylphenyl)-5H-pyrrolo[2,3-b]pyrazine (160 mg, 0.72 mmol) under N2. The resulting mixture was stirred at 105° C. for 2 hr under N2. Upon completion, the resulting mixture was concentrated in vacuo. The residue was diluted with THF, and successively washed with brine, 1 M HCl, and dried over anhydrous sodium sulfate, and concentrated in vacuo. The residue was purified by Prep-TLC (DCM/MeOH, 10:1) to obtain methyl 2-(azetidin-3-ylmethyl)-4-[3-(3,5-dimethylphenyl)pyrrolo[2,3-b]pyrazin-5-yl]-benzoate (200 mg, 66.8% yield) as an off-white semi-solid. MS:[M+H]=427.

STEP C. To a stirred mixture of methyl 2-(azetidin-3-ylmethyl)-4-[3-(3, 5-dimethylphenyl)-pyrrolo[2,3-b]pyrazin-5-yl]benzoate (190 mg, 0.45 mmol) in THF (2 mL), methanol (2 mL) and Water (1 mL) was added NaOH (150 mg, 3.75 mmol). The resulting mixture was stirred at 50° C. for 1 h. The resulting mixture was concentrated. The residue was added H₂O, adjusted to pH˜5 with 1 N HCl, and extracted with THF. The extracts were dried over anhydrous sodium sulfate, filtered and concentrated. The residue was purified by Prep-HPLC to obtain 2-(azetidin-3-ylmethyl)-4-[3-(3,5-dimethylphenyl) pyrrolo[2,3-b]pyrazin-5-yl]benzoic acid (23.2 mg, 12.5% yield) as an off-white solid. [M+H] Calc'd for C₂₅H₂₄N₄O₂, 413. Found, 413. ¹H NMR (400 MHz, CF₃COOD) δ 9.05 (s, 1H), 8.54-8.63 (m, 2H), 8.08 (d, J=8.6 Hz, 1H), 7.98 (s, 1H), 7.73 (s, 2H), 7.49 (s, 1H), 7.26-7.35 (m, 2H), 4.47 (m, 4H), 3.73-3.83 (m, 3H), 2.47 (s, 6H). Prep-HPLC conditions [(Column: XBridge Prep OBD C18 Column 30×150 mm 5 um; Mobile Phase A: Water (10 MMOL/L NH₄HCO₃), Mobile Phase B: ACN; Flow rate: 60 mL/min; Gradient: 35% B to 95% B in 7.5 min; 254/210 nm; Rt: 7.1 min).

### Example 28: (R) and (S)-4-(3-(3,5-dimethylphenyl)-5H-pyrrolo[2,3-b]pyrazin-5-yl)-2-(piperidin-3-yl) benzoic Acid

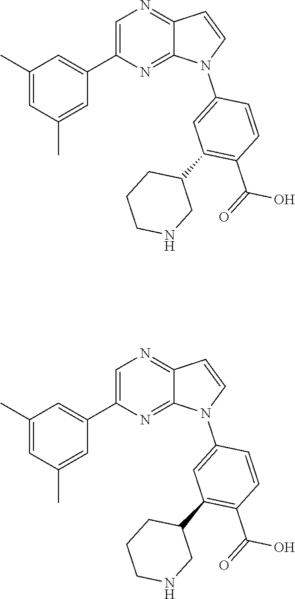

STEP A. To a stirred solution of methyl 2-bromo-4-nitro-benzoate (2.5 g, 9.61 mmol), tert-butyl-5-(4,4,5,5-tetramethyl-1,3,2-dioxaborolan-2-yl)-3,6-dihydro-2H-pyridine-1-carboxylate (3 g, 9.70 mmol) in 1,4-Dioxane (50 mL) and Water (10 mL) was added Na₂CO₃ (3.1 g, 28.8 mmol) and Pd(dppf)Cl₂ (0.66 g, 0.97 mmol). The resulting mixture was purged and backfilled with N₂ for 3 times. The resulting mixture was stirred at 80° C. for 3 h under N₂. Upon completion, the reaction mixture was diluted with EA and washed with water. The organic layers were dried over Na₂SO₄, filtered and concentrated in vacuo. The residue was purified by silica gel column chromatography (PE/EA=10:1) to obtain tert-butyl 5-(2-methoxycarbonyl-5-nitro-phenyl)-3,6-dihydro-2H-pyridine-1-carboxylate (3.2 g, 91.8% yield) as a light yellow solid. MS: [M+H]=363.

STEP B. To a well stirred solution of tert-butyl 5-(2-methoxycarbonyl-5-nitri-phenyl)-3,6-dihydro-2H-pyridine-1-carboxylate (3.1 g, 8.55 mmol) in MeOH (50 mL) was added Pd/C (3.1 g, 29 mmol) under a nitrogen atmosphere. The reaction mixture was purged and backfilled with H₂. The reaction was stirred at room temperature overnight. Upon completion, the mixture was filtered, the filtrate was concentrated in vacuo to furnish tert-butyl 5-(5-amino-2-methoxycarbonyl-phenyl)-3, 6-dihydro-2H-pyridine-1-carboxylate (2.5 g, 87.9% yield) as an colorless oil which was used in the next step without further purification. MS: [M+H]=333.

STEP C. To a well stirred solution of tert-butyl 5-(5-amino-2-methoxycarbonyl-phenyl)-3,6-dihydro-2H-pyridine-1-carboxylate (2.4 g, 7.22 mmol) in ethyl acetate (50 mL) was added PtO₂ (1.2 g, 5.2 mmol) and HOAc (2 mL, 7.22 mmol) at the room temperature under N₂. The reaction mixture was purged and backfilled with H₂ and allowed to stir at room temperature overnight. Upon completion, the reaction mixture was filtered, and the filtrate cake washed with EA. The combined filtrate was concentrated in vacuo to afford tert-butyl 3-(5-amino-2-methoxycarbonyl-phenyl) piperidine-1-carboxylate as off-white oil which was used without further purification. MS: [M+H]=335.

STEP D. To a stirred solution of tert-butyl 3-(5-amino-2-methoxycarbonyl-phenyl)piperidine-1-carboxylate (2 g, 5.83 mmol) in MeCN was added tert-butyl nitrite (970 mg, 9.46 mmol) and CuBr (1.3 g, 9.52 mmol) (30 mL) at 0° C. The resulting mixture was stirred at room temperature for 1.5 h. Then the reaction mixture was concentrated. The residue was added water and extracted with EA. The extracts were washed with brine, dried over Na₂SO₄, concentrated. The crude product was passed through silica gel column (PE/EA, 5:1 to 1:1) to obtain tert-butyl 3-(5-bromo-2-methoxycarbonyl-phenyl) piperidine-1-carboxylate (1.68 g, 72.3% yield) as a colorless oil. MS: [M+H]=398. The tert-butyl 3-(5-bromo-2-methoxycarbonyl-phenyl)piperidine-1-carboxylate (1.68 g, 4.2 mmol) was purified by Prep-SFC with the following conditions: Column: (R,R)-WHELK-O1-Kromasil (02), 5 cm*25 cm (5 um); Mobile Phase A: CO₂:75, Mobile Phase B: MeOH (0.1% DEA); Flow rate: 40 mL/min; 220 nm; RT1: 4.81; RT2: 6.38. Optically pure fractions were combined and evaporated to obtain Peak A (520 mg) and Peak B (600 mg) as colorless oil. MS: [M+H]=398. Peak A: ¹H NMR (400 MHz, Methanol-d₄) δ 7.70 (d, J=8.4 Hz, 2H), 7.62 (d, J=2.0 Hz, 1H), 7.50 (dd, J=8.4, 2.0 Hz, 1H), 4.19-4.07 (m, 2H), 3.86 (s, 3H), 3.49 (s, 1H), 2.87 (s, 2H), 1.98 (d, 1H), 1.78 (s, 2H), 1.72 (s, 2H), 1.65-1.52 (m, 1H), 1.49 (s, 9H). Peak B: ¹H NMR (400 MHz, Methanol-d₄) δ 7.70 (d, J=8.4 Hz, 1H), 7.68 (d, J=2.0 Hz, 1H), 7.49 (dd, J=8.4, 2.0 Hz, 1H), 4.14 (m, J=12.7, 4.0, 1.7 Hz, 2H), 3.89 (s, 3H), 3.47 (s, 1H), 2.84 (s, 2H), 1.96 (d, 1H), 1.78-1.75 (m, 2H), 1.61-1.52 (m, 1H), 1.46 (s, 9H).

STEP E. To a stirred solution of tert-butyl 3-(5-bromo-2-(methoxycarbonyl)phenyl)piperidine-1-carboxylate (150 mg, 0.38 mmol, Peak A) in DMA (5 mL) were added CuI (14 mg, 0.07 mmol), Cs₂CO₃ (245 mg, 0.75 mmol), 2-isobutyrylcyclohexan-1-one (25 mg, 0.15 mmol) and 3-(3,5-dimethylphenyl)-5H-pyrrolo[2,3-b] pyrazine (85 mg, 0.38 mmol) at room temperature under N₂. The resulting mixture was stirred at 110° C. for 3 h. Upon completion, the resulting mixture was concentrated in vacuo and the residue dissolved in DMSO and purified by reverse-phase chromatography. Pure fractions were concentrated in vacuo to obtain tert-butyl 3-(5-(3-(3,5-dimethylphenyl)-5H-pyrrolo[2,3-b]pyrazin-5-yl)-2-(methoxycarbonyl)phenyl)piperidine-1-carboxylate (120 mg, 58.9% yield) as a yellow solid. MS: [M+H]=541.

STEP F. To a stirred solution of tert-butyl 3-(5-(3-(3,5-dimethylphenyl)-5H-pyrrolo[2,3-b]pyrazin-5-yl)-2-(methoxycarbonyl)phenyl)piperidine-1-carboxylate (120 mg, 0.22 mmol) in water (2 mL), THF (5 mL) and MeOH (5 mL) was added NaOH (50 mg, 1.25 mmol). The resulting mixture was stirred at 50° C. for 1 h. Upon completion, the reaction mixture was concentrated in vacuo and water was added (1 mL). The pH of the solution was adjusted to ˜5 with sat. Citric acid, and extracted with THF. The organic layers were successively washed with brine, dried over anhydrous sulfate sodium, and concentrated in vacuo to obtain optically 2-(1-(tert-butoxycarbonyl)piperidin-3-yl)-4-(3-(3,5-dimethylphenyl)-5H-pyrrolo[2,3-b]pyrazin-5-yl)benzoic acid (110 mg, 94.1% yield) as a yellow solid. MS: [M+H]=527.

STEP G. To a stirred solution of 2-(1-(tert-butoxycarbonyl)piperidin-3-yl)-4-(3-(3,5-dimethylphenyl)-5H-pyrrolo[2,3-b]pyrazin-5-yl)benzoic acid (150 mg, 0.28 mmol) in DCM (2 mL) was added TFA (1 mL, 12.3 mmol) at room temperature and the resulting solution was stirred for 20 min. The resulting solution was concentrated and purified by Prep-HPLC to obtain the title compound (65.7 mg, 53.5% yield) as a light yellow solid. [M+H] Calc'd for C₂₆H₂₆N₄O₂, 427. Found, 427. ¹H NMR (400 MHz, Methanol-d₄) δ 8.97 (s, 1H), 8.30 (d, J=2.2 Hz, 1H), 8.26 (d, J=3.9 Hz, 1H), 8.25 (d, J=8.6 Hz, 1H), 7.93 (dd, J=8.6, 2.2 Hz, 1H), 7.75 (d, J=1.6 Hz, 2H), 7.16-7.11 (m, 1H), 6.90 (d, J=3.9 Hz, 1H), 4.25 (tt, J=12.0, 3.4 Hz, 1H), 3.68-3.59 (m, 1H), 3.46 (d, J=13.0 Hz, 1H), 3.14-2.99 (m, 2H), 2.40 (s, 6H), 2.24 (d, J=12.1 Hz, 1H), 2.14-2.07 (m, 2H), 2.08-1.90 (m, 1H). Prep-HPLC conditions [Column: Xselect CSH OBD Column 30*150 mm 5 um n; Mobile Phase A: Water (0.05% TFA), Mobile Phase B: ACN; Flow rate: 60 mL/min; Gradient: 30% B to 40% B in 7 min; 254/210 nm; Rt: 6.2 min.].

The title compound was prepared in 23% yield starting from STEP D using tert-butyl 3-(5-bromo-2-methoxycarbonyl-phenyl)piperidine-1-carboxylate (PEAK B). [M+H] Calc'd for C₂₆H₂₆N₄O₂, 427. Found, 427. ¹H NMR (400 MHz, Methanol-d₄) δ 8.97 (s, 1H), 8.30 (d, J=2.2 Hz, 1H), 8.26 (d, J=3.9 Hz, 1H), 8.25 (d, J=8.6 Hz, 1H), 7.93 (dd, J=8.6, 2.2 Hz, 1H), 7.75 (d, J=1.6 Hz, 2H), 7.16-7.11 (m, 1H), 6.90 (d, J=3.9 Hz, 1H), 4.25 (tt, J=12.0, 3.4 Hz, 1H), 3.68-3.59 (m, 1H), 3.46 (d, J=13.0 Hz, 1H), 3.14-2.99 (m, 2H), 2.40 (s, 6H), 2.24 (d, J=12.1 Hz, 1H), 2.14-2.07 (m, 2H), 2.08-1.90 (m, 1H). Prep-HPLC conditions [Column: Xselect CSH OBD Column 30*150 mm 5 um n; Mobile Phase A: Water (0.05% TFA), Mobile Phase B: ACN; Flow rate: 60 mL/min; Gradient: 30% B to 40% B in 7 min; 254/210 nm; Rt: 6.2 min.]

### Example 29. 4-(3-(3,5-dimethylphenyl)-5H-pyrrolo[2,3-b]pyrazin-5-yl)-2-(piperidin-4-yl)benzoic Acid

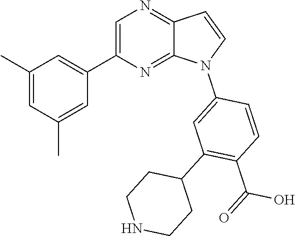 [SMILES: Cc1cc(C)cc(-c2cnc3ccn(-c4ccc(C(=O)O)c(C5CCNCC5)c4)c3n2)c1]

The title compound was prepared in 6.9% overall yield as a light-yellow solid according to the preparation for Example 27. MS: [M+H] Calc'd for C₂₆H₂₆N₄O₂, 427. Found, 427. ¹H NMR (400 MHz, MeOD) δ 9.04 (s, 1H), 8.26 (t, J=3.4 Hz, 2H), 8.20 (d, J=8.5 Hz, 1H), 7.85 (dd, J=8.5, 2.2 Hz, 1H), 7.82 (d, J=1.6 Hz, 2H), 7.12 (m, 1H), 6.92 (d, J=3.9 Hz, 1H), 4.00-4.15 (m, 1H), 3.50-3.57 (m, 2H), 3.18-3.24 (m, 2H), 2.41 (s, 6H), 2.28 (m, 2H), 2.01-2.13 (m, 2H). Prep-HPLC conditions [Column: Sunfire prep C18 column 30*150, Sum; Mobile Phase A: Water (0.05% TFA), Mobile Phase B: ACN; Flow rate: 60 mL/min; Gradient: 25% B to 45% B in 7 min; 254/210 nm; Rt: 5.37 min.]

### Example 30. 2-((azetidin-3-ylmethyl)amino)-4-(3-(3,5-dimethylphenyl)-5H-pyrrolo[2,3-b]pyrazin-5-yl)benzoic Acid

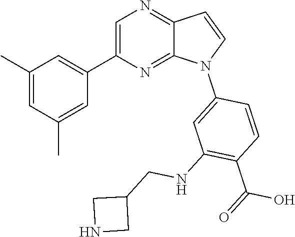 [SMILES: Cc1cc(C)cc(-c2cnc3ccn(-c4ccc(C(=O)O)c(NCC5CNC5)c4)c3n2)c1]

The title compound was prepared in 26.4% overall yield as a yellow solid according to the preparation for Example 25. [M+H] Calc'd for C₂₅H₂₅N₅O₂, 428. Found, 428. ¹H NMR (400 MHz, Methanol-d₄) δ 8.94 (s, 1H), 8.25 (d, J=3.9 Hz, 1H), 8.12 (d, J=8.7 Hz, 1H), 7.72-7.67 (m, 3H), 7.20 (dd, J=8.7, 2.1 Hz, 1H), 7.15 (d, J=1.8 Hz, 1H), 6.87 (d, J=3.9 Hz, 1H), 4.05 (dd, J=11.1, 8.9 Hz, 2H), 3.94 (dd, J=10.9, 7.6 Hz, 2H), 3.66 (d, J=6.6 Hz, 2H), 3.37-3.33 (m, 1H), 2.41 (s, 6H). Prep-HPLC conditions [Column: Xselect CSH OBD Column 30*150 mm 5 um; Mobile Phase A: Water (0.05% TFA), Mobile Phase B: ACN; Flow rate: 60 mL/min; Gradient: 30% B to 40% B in 7 min; 210/254 nm; Rt: 5.75 min]

### Example 31. (R)-4-(3-(3,5-dimethylphenyl)-5H-pyrrolo[2,3-b]pyrazin-5-yl)-2-(pyrrolidin-3-ylamino)benzoic Acid

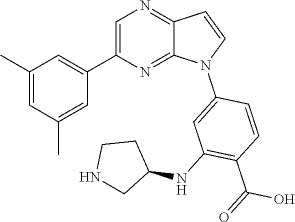 [SMILES: Cc1cc(C)cc(-c2cnc3ccn(-c4ccc(C(=O)O)c(N[C@@H]5CCNC5)c4)c3n2)c1]

The title compound was prepared in 16% overall yield as a yellow solid according to the preparation for Example 25. [M+H] Calc'd for C₂₅H₂₅N₅O₂, 428. Found, 428. ¹H NMR (300 MHz, Methanol-d4) δ 8.93 (s, 1H), 8.23 (d, J=3.9 Hz, 1H), 8.15 (d, J=8.7 Hz, 1H), 7.69 (s, 2H), 7.61 (d, J=1.9 Hz, 1H), 7.19 (dd, J=8.6, 2.0 Hz, 1H), 7.12 (s, 1H), 6.87 (d, J=3.8 Hz, 1H), 4.46 (s, 1H), 3.56-3.31 (m, 4H), 2.48 (s, 8H), 2.25 (s, 1H). Prep-HPLC conditions [Column: XBridge Shield RP18 OBD Column 19*250 mm, 10 um; Mobile Phase A: Water (10 MMOL/L NH4HCO3+0.1% NH3·H2O), Mobile Phase B: ACN; Flow rate: 25 mL/min; Gradient: 24% B to 50% B in 7 min; 254/210 nm; Rt: 6.80 min]

### Example 32. (S)-4-(3-(3,5-dimethylphenyl)-5H-pyrrolo[2,3-b]pyrazin-5-yl)-2-(pyrrolidin-3-ylamino)benzoic Acid

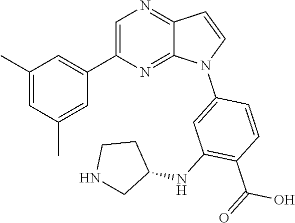 [SMILES: Cc1cc(C)cc(-c2cnc3ccn(-c4ccc(C(=O)O)c(N[C@H]5CCNC5)c4)c3n2)c1]

The title compound was prepared in 21.9% overall yield as a yellow solid according to the preparation for Example 25. [M+H] Calc'd for C₂₅H₂₅N₅O₂, 428. Found, 428. ¹H NMR (400 MHz, DMSO-d6) δ 8.17 (s, 1H), 7.46 (d, J=3.9 Hz, 1H), 7.36 (d, J=8.6 Hz, 1H), 6.93 (s, 2H), 6.84 (d, J=2.1 Hz, 1H), 6.44 (dd, J=8.6, 2.1 Hz, 1H), 6.34 (s, 1H), 6.10 (d, J=3.9 Hz, 1H), 3.69 (s, 1H), 2.73 (td, J=10.4, 9.0, 5.7 Hz, 2H), 2.64 (dd, J=12.6, 8.8 Hz, 2H), 1.65 (s, 7H), 1.62 (s, 1H). Prep-HPLC conditions [Column: Xselect CSH OBD Column 30*150 mm 5 um n; Mobile Phase A: Water (0.05% TFA), Mobile Phase B: ACN; Flow rate: 60 mL/min; Gradient: 25% B to 43% B in 7 min; 210/254 nm; Rt: 6.17 min]

### Example 33. (R)-4-(3-(3,5-dimethylphenyl)-5H-pyrrolo[2,3-b]pyrazin-5-yl)-2-(piperidin-3-ylamino)benzoic Acid

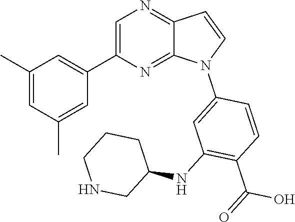 [SMILES: Cc1cc(C)cc(-c2cnc3ccn(-c4ccc(C(=O)O)c(N[C@@H]5CCCNC5)c4)c3n2)c1]

The title compound was prepared in 10.3% overall yield as a light yellow solid according to the preparation for Example 25. [M+H] Calc'd for C₂₆H₂₇N₅O₂, 442. Found, 442. ¹H NMR (400 MHz, Methanol-d4) δ 8.96 (s, 1H), 8.21 (d, J=3.9 Hz, 1H), 8.16 (d, J=8.6 Hz, 1H), 7.73 (s, 2H), 7.44 (d, J=2.1 Hz, 1H), 7.28-7.26 (m, 1H), 7.12 (s, 1H), 6.88 (d, J=3.9 Hz, 1H), 3.97-3.93 (m, 1H), 3.48-3.43 (m, 1H), 3.32-3.24 (m, 1H), 3.10-3.03 (m, 2H), 2.40 (s, 6H), 2.30 (t, 1H), 2.08-1.96 (m, 1H) 1.96-1.74 (m, 2H). Prep-HPLC conditions [Column: X select CSH OBD Column 30*150 mm 5 um n; Mobile Phase A: Water (0.05% TFA), Mobile Phase B: ACN; Flow rate: 60 mL/min; Gradient: 25% B to 46% B in 7 min; 210-254 nm; R t: 6.25 min]

### Example 34. 4-(3-(3,5-dimethylphenyl)-5H-pyrrolo[2,3-b]pyrazin-5-yl)-2-(piperidin-4-ylamino)benzoic Acid

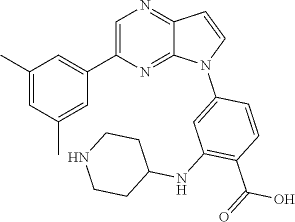 [SMILES: Cc1cc(C)cc(-c2cnc3ccn(-c4ccc(C(=O)O)c(NC5CCNCC5)c4)c3n2)c1]

The title compound was prepared in 51.5% overall yield as a light yellow solid according to the preparation for Example 25. [M+H] Calc'd for C₂₆H₂₇N₅O₂, 442. Found, 442. ¹H NMR (400 MHz, Methanol-d₄) δ 8.90 (s, 1H), 8.20 (d, J=3.9 Hz, 1H), 8.12 (d, J=8.6 Hz, 1H), 7.65 (dt, J=1.6, 0.7 Hz, 2H), 7.58 (d, J=2.1 Hz, 1H), 7.13 (tt, J=1.6, 0.8 Hz, 1H), 7.08 (dd, J=8.6, 2.1 Hz, 1H), 6.86 (d, J=3.9 Hz, 1H), 3.82 (td, J=9.3, 4.5 Hz, 1H), 3.25 (m, 2H), 2.98 (m, 2H), 2.45-2.36 (m, 8H), 1.80 (m, 2H). Prep-HPLC conditions [Column: Xselect CSH OBD Column 30*150 mm 5 um; Mobile Phase A: Water (0.05% TFA), Mobile Phase B: ACN; Flow rate: 60 mL/min; Gradient: 30% B to 38% B in 7 min; 210-254 nm; Rt: 5.82 min]

### Example 35. (R)-4-(3-(3,5-dimethylphenyl)-5H-pyrrolo[2,3-b]pyrazin-5-yl)-2-(pyrrolidin-3-yloxy)benzoic Acid

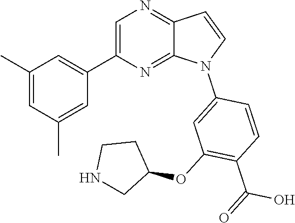 [SMILES: Cc1cc(C)cc(-c2cnc3ccn(-c4ccc(C(=O)O)c(O[C@@H]5CCNC5)c4)c3n2)c1]

The title compound was prepared in 34.1% overall yield as a yellow solid according to the preparation for Example 24. [M+H] Calc'd for C₂₅H₂₄N₄O₃, 429. Found 429. ¹H NMR (400 MHz, Methanol-d₄) δ 8.95 (s, 1H), 8.27 (s, 1H), 8.10 (d, J=7.1 Hz, 2H), 7.69 (s, 3H), 7.13 (s, 1H), 6.90 (s, 1H), 5.43 (s, 1H), 3.75 (d, J=12.7 Hz, 1H), 3.50 (s, 1H), 3.34 (d, J=13.2 Hz, 2H), 2.53 (s, 1H), 2.41 (q, J=3.4 Hz, 6H), 2.33 (s, 1H). Prep-HPLC conditions [Column: Sunfire prep C18 column 30*150, Sum; mobile Phase A: Water (0.05% TFA), Mobile Phase B: ACN; Flow rate: 60 mL/min; Gradient: 20% B to 44% B in 8 min; 254 nm; Rt: 6.70 min]

### Example 2.1: 2-Cyclopentyl-4-(2-cyclopropylpyrazolo[1,5-a]pyrimidin-7-yl)benzoic Acid

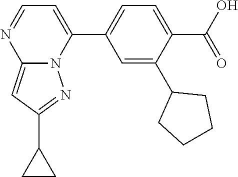 [SMILES: O=C(O)c1ccc(-c2ccnc3cc(C4CC4)nn23)cc1C1CCCC1]

### Preparation 2.1A: 4-Bromo-2-cyclopentylbenzoic Acid

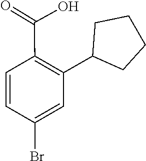 [SMILES: O=C(O)c1ccc(Br)cc1C1CCCC1]

To a stirred solution of 4-bromo-2-fluoro-benzoic acid (10 g, 45.66 mmol) in THF (100 mL) in a 500-mL 3-necked round-bottom flask was added bromo(cyclopentyl)magnesium (114 mL, 114.15 mmol) drop wise at 0° C. under a nitrogen atmosphere. The resulting mixture was warmed up to room temperature and stirred overnight. The mixture was quenched by water at 0° C. and concentrated under reduced pressure. The mixture was filtered and the filtrate was collected. The pH value of the solution was adjusted to 3 with HCl (1 mol/L). The mixture was filtered. The filter cake was collected and concentrated under vacuum to give 4-bromo-2-cyclopentyl-benzoic acid (7.25 g, 59%) as an off-white solid. [M−H] Calc'd for C₁₂H₁₃BO₂, 268. Found, 267/269.

### Preparation 2.1B: 2-Cyclopentyl-4-(4,4,5,5-tetramethyl-1,3,2-dioxaborolan-2-yl)benzoic Acid

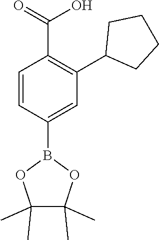 [SMILES: CC1(C)OB(c2ccc(C(=O)O)c(C3CCCC3)c2)OC1(C)C]

To a stirred solution of 4-bromo-2-cyclopentyl-benzoic acid (7.25 g, 26.94 mmol), 4,4,5,5-tetramethyl-2-(4,4,5,5-tetramethyl-1,3,2-dioxaborolan-2-yl)-1,3,2-dioxaborolane (10.26 g, 40.41 mmol) and KOAc (7.93 g, 80.82 mmol) in 1,4-dioxane (100 mL) was added Pd(dppf)Cl₂ (1.97 g, 2.90 mmol). The resulting mixture was stirred for overnight at 80° C. under a nitrogen atmosphere. LCMS showed the reaction was complete and the reaction mixture was concentrated under vacuum. The residue was diluted with ethyl acetate and filtered. The filtrate was collected and concentrated under vacuum. The obtained mixture was purified by flash column chromatography (PE/EA=9/1) to afford 2-cyclopentyl-4-(4,4,5,5-tetramethyl-1,3,2-dioxaborolan-2-yl)benzoic acid (7.8 g, 92%) as a yellow solid. ¹H NMR (400 MHz, DMSO-d₆) δ 12.99 (s, 1H), 7.66 (s, 1H), 7.58-7.59 (m, 2H), 3.62-3.57 (m, 1H), 1.97 (s, 2H), 1.76 (s, 2H), 1.60-1.48 (m, 4H), 1.27 (s, 12H). [M+H] Calc'd for C₁₈H₂₅BO₄, 317. Found, 317.

### Preparation 2.1C: 4-(2-Bromopyrazolo[1,5-a]pyrimidin-7-yl)-2-cyclopentylbenzoic Acid

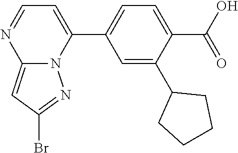 [SMILES: O=C(O)c1ccc(-c2ccnc3cc(Br)nn23)cc1C1CCCC1]

To a stirred solution of 2-bromo-7-chloro-pyrazolo[1,5-a]pyrimidine (2200 mg, 9.46 mmol) in 1,4-dioxane (50 mL) was added 2-cyclopentyl-4-(4,4,5,5-tetramethyl-1,3,2-dioxaborolan-2-yl)benzoic acid (4498 mg, 14.19 mmol, Preparation 2.1B), and saturated Na₂CO₃ solution (10 mL) under nitrogen. Pd(dppf)Cl₂ (252 mg, 0.34 mmol) was then added to above mixture under N₂. The resulting mixture was stirred at 80° C. for 2 hours. LCMS showed completion of the reaction. The reaction mixture was diluted with ethyl acetate (3×200 mL) and washed with water; the combined organic layers were dried over Na₂SO₄, concentrated. The obtained residue was purified by silica gel to afford 4-(2-bromopyrazolo[1,5-a]pyrimidin-7-yl)-2-cyclopentyl-benzoic acid (2 g, 55%) as a yellow solid. [M+H] Calc'd for C₁₈H₁₆BrN₃O₂, 387. Found, 386/388.

To a stirred solution of 4-(2-bromopyrazolo[1,5-a]pyrimidin-7-yl)-2-cyclopentyl-benzoic acid (150 mg, 0.39 mmol, Preparation 2.1C) in 1,4-dioxane (10 mL) was added cyclopropylboronic acid (50 mg, 0.58 mmol), saturated Na₂CO₃ solution (2 mL) and Pd(dppf)Cl₂ (57 mg, 0.08 mmol) under nitrogen. The resulting mixture was stirred at 80° C. for 2 hours and purified by reverse phase column to give a crude product. Further HPLC purification afforded 2-cyclopentyl-4-(2-cyclopropylpyrazolo[1,5-a]pyrimidin-7-yl)benzoic acid (12 mg, 9%) as a yellow solid. ¹H NMR (300 MHz, DMSO-d₆) δ 8.52 (d, J=4.4 Hz, 1H), 8.25 (d, J=1.8 Hz, 1H), 7.88 (dd, J=8.1, 1.7 Hz, 1H), 7.70 (d, J=8.1 Hz, 1H), 6.59 (s, 1H), 3.78 (t, J=8.2 Hz, 1H), 2.20-1.95 (m, 2H), 1.81 (s, 2H), 1.74-1.52 (m, 5H), 1.13-0.98 (m, 2H), 0.98-0.83 (m, 2H). [M+H] Calc'd for C₂₁H₂₁N₃O₂, 348. Found, 348.

### Example 2.2: 2-Cyclopentyl-4-(2-phenylpyrazolo[1,5-a]pyrimidin-7-yl)benzoic Acid

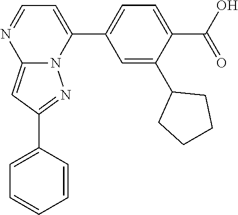 [SMILES: O=C(O)c1ccc(-c2ccnc3cc(-c4ccccc4)nn23)cc1C1CCCC1]

To a stirred solution of 4-(2-bromopyrazolo[1,5-a]pyrimidin-7-yl)-2-cyclopentyl-benzoic acid (150 mg, 0.39 mmol, Preparation 2.1C) in 1,4-dioxane (10 mL) was added phenylboronic acid (71 mg, 0.58 mmol), saturated Na₂CO₃ solution (2 mL), and Pd(dppf)Cl₂ (57 mg, 0.08 mmol) under nitrogen. The resulting mixture was stirred at 80° C. for 2 hours and purified by reverse phase column to give a crude product. Further HPLC purification afforded 2-cyclopentyl-4-(2-phenylpyrazolo[1,5-a]pyrimidin-7-yl)benzoic acid (33 mg, 22%) as a yellow solid. ¹H NMR (400 MHz, DMSO-d₆) δ 8.59 (d, J=4.4 Hz, 1H), 8.35 (d, J=1.8 Hz, 1H), 8.10-8.02 (m, 2H), 7.91 (dd, J=8.0, 1.8 Hz, 1H), 7.61 (d, J=8.0 Hz, 1H), 7.49 (dd, J=8.2, 6.5 Hz, 2H), 7.46-7.38 (m, 1H), 7.34 (s, 1H), 7.29 (d, J=4.4 Hz, 1H), 3.82 (t, J=8.5 Hz, 1H), 2.14-2.03 (m, 2H), 1.80 (d, J=12.3 Hz, 2H), 1.67 (qd, J=9.8, 9.1, 4.8 Hz, 4H). [M+H] Calc'd for C₂₄H₂₁N₃O₂, 384. Found, 384.

### Example 2.3: 2-Cyclopentyl-4-(2-methoxypyrazolo[1,5-a]pyrimidin-7-yl)benzoic Acid

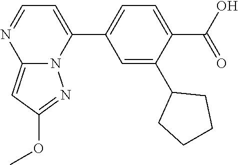 [SMILES: COc1cc2nccc(-c3ccc(C(=O)O)c(C4CCCC4)c3)n2n1]

To a stirred solution of 4-(2-bromopyrazolo[1,5-a]pyrimidin-7-yl)-2-cyclopentyl-benzoic acid (150 mg, 0.39 mmol) in 1,4-dioxane (5 mL) and MeOH (5 mL) was added KOH (66 mg, 1.16 mmol), Pd₂(dba)₃ (81 mg, 0.08 mmol) and t-Bu-Brettphos (36 mg, 0.08 mmol) under nitrogen. The resulting mixture was stirred at 100° C. for 4 hours and purified by reverse phase column to give a crude product. Further HPLC purification afforded 2-cyclopentyl-4-(2-methoxypyrazolo[1,5-a]pyrimidin-7-yl)benzoic acid (17 mg, 13%) as a yellow solid. ¹H NMR (400 MHz, DMSO-d₆) δ 8.50 (d, J=4.5 Hz, 1H), 8.22 (d, J=1.8 Hz, 1H), 7.86 (dd, J=8.1, 1.8 Hz, 1H), 7.67 (d, J=8.1 Hz, 1H), 7.15 (d, J=4.5 Hz, 1H), 6.23 (s, 1H), 3.95 (s, 3H), 3.76 (t, J=8.3 Hz, 1H), 2.05 (s, 2H), 1.83-1.76 (m, 2H), 1.69-1.58 (m, 1H), 1.64 (s, 4H). [M+H] Calc'd for C₁₉H₁₉N₃O₃, 338. Found, 338.

### Example 2.4: 2-Cyclopentyl-4-(2-ethoxypyrazolo[1,5-a]pyrimidin-7-yl)benzoic Acid

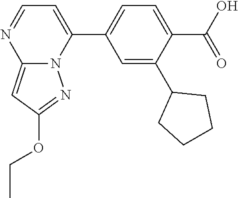 [SMILES: CCOc1cc2nccc(-c3ccc(C(=O)O)c(C4CCCC4)c3)n2n1]

The title compound was prepared in 24% yield according to the general procedure for the preparation of Example 2.3 using ethanol. ¹H NMR (300 MHz, DMSO-d₆) δ 8.49 (d, J=4.5 Hz, 1H), 8.21 (d, J=1.8 Hz, 1H), 7.86 (dd, J=8.1, 1.8 Hz, 1H), 7.67 (d, J=8.1 Hz, 1H), 7.15 (d, J=4.6 Hz, 1H), 6.21 (s, 1H), 4.31 (q, J=7.0 Hz, 2H), 3.78 (q, J=8.4 Hz, 1H), 2.05 (s, 3H), 1.80 (s, 3H), 1.64 (s, 5H), 1.38 (t, J=7.0 Hz, 3H). [M+H] Calc'd for C₂₀H₂₁N₃O₃, 352. Found, 352.

### Example 2.5: 2-Cyclopentyl-4-(2-(4-fluorophenyl)pyrazolo[1,5-a]pyrimidin-7-yl)benzoic Acid

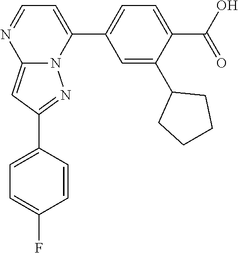 [SMILES: O=C(O)c1ccc(-c2ccnc3cc(-c4ccc(F)cc4)nn23)cc1C1CCCC1]

The title compound was prepared in 50% yield according to the general procedure for the preparation of Example 2.2 using (4-fluorophenyl)boronic acid. ¹H-NMR (400 MHz, DMSO-d₆) δ=8.61 (d, 1H), 8.35 (s, 1H), 8.07-8.11 (m, 2H), 7.99 (d, 1H), 7.78 (d, 1H), 7.31-7.36 (m, 4H), 3.81 (m, 1H), 1.82 (d, 2H), 1.76-1.77 (d, 2H), 1.67-1.68 (d, 4H). [M+H] Calc'd for C₂₄H₂₀FN₃O₂, 402. Found, 402.

### Example 2.6: 2-Cyclopentyl-4-(2-(2-fluorophenyl)pyrazolo[1,5-a]pyrimidin-7-yl)benzoic Acid

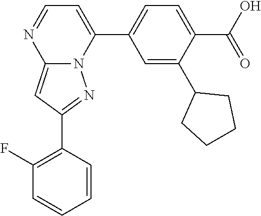 [SMILES: O=C(O)c1ccc(-c2ccnc3cc(-c4ccccc4F)nn23)cc1C1CCCC1]

The title compound was prepared in 45% yield according to the general procedure for the preparation of Example 2.2 using (2-fluorophenyl)boronic acid. ¹H NMR (400 MHz, DMSO-d₆) δ=13.44 (s, 1H), 8.67 (d, 1H), 8.42 (s, 1H), 8.10-8.11 (m, 2H), 7.85 (d, 1H), 7.31-7.36 (m, 4H), 7.16 (d, 1H), 3.80 (m, 1H), 2.09-2.11 (d, 2H), 1.75-1.83 (d, 6H). [M+H] Calc'd for C₂₄H₂₀FN₃O₂, 402. Found, 402.

### Example 2.7: 2-Cyclopentyl-4-(2-(3-fluorophenyl)pyrazolo[1,5-a]pyrimidin-7-yl)benzoic Acid

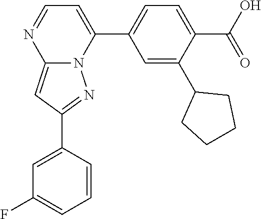 [SMILES: O=C(O)c1ccc(-c2ccnc3cc(-c4cccc(F)c4)nn23)cc1C1CCCC1]

The title compound was prepared in 51% yield according to the general procedure for the preparation of Example 2.2 using (3-fluorophenyl)boronic acid. ¹H NMR (400 MHz, DMSO-d₆) δ=8.64 (d, 1H), 8.44 (d, 1H), 8.01 (m, 1H), 7.81-7.92 (m, 3H), 7.82 (m, 1H), 7.57 (s, 1H), 7.56 (d, 1H), 7.25 (m, 1H), 3.81 (m, 1H), 2.08-2.09 (d, 2H), 1.81-1.83 (d, 2H), 1.67-1.69 (d, 4H). [M+H] Calc'd for C₂₄H₂₀FN₃O₂, 402. Found, 402.

### Example 2.8: 2-Cyclopentyl-4-(2-(4-methoxyphenyl)pyrazolo[1,5-a]pyrimidin-7-yl)benzoic Acid

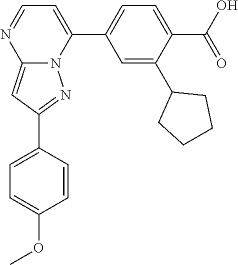 [SMILES: COc1ccc(-c2cc3nccc(-c4ccc(C(=O)O)c(C5CCCC5)c4)n3n2)cc1]

The title compound was prepared in 17% yield according to the general procedure for the preparation of Example 2.2 using (4-methoxyphenyl)boronic acid. ¹H NMR (400 MHz, DMSO-d₆) δ 8.59 (d, J=4.4 Hz, 1H), 8.42 (d, J=1.7 Hz, 1H), 8.04-7.94 (m, 3H), 7.80 (d, J=8.1 Hz, 1H), 7.33-7.24 (m, 2H), 7.11-7.01 (m, 2H), 3.83 (s, 3H), 2.11 (s, 2H), 1.84 (s, 2H), 1.73-1.65 (m, 5H). [M+H] Calc'd for C₂₅H₂₃N₃O₃, 414. Found, 414.

### Example 2.9: 2-Cyclopentyl-4-(2-(3-methoxyphenyl)pyrazolo[1,5-a]pyrimidin-7-yl)benzoic Acid

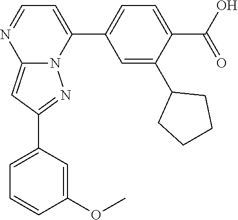 [SMILES: COc1cccc(-c2cc3nccc(-c4ccc(C(=O)O)c(C5CCCC5)c4)n3n2)c1]

The title compound was prepared in 17% yield according to the general procedure for the preparation of Example 2.2 using (3-methoxyphenyl)boronic acid. ¹H NMR (400 MHz, DMSO-d₆) δ 8.62 (d, J=4.4 Hz, 1H), 8.47 (d, J=1.8 Hz, 1H), 7.97 (dd, J=8.0, 1.7 Hz, 1H), 7.79 (d, J=8.1 Hz, 1H), 7.70-7.58 (m, 2H), 7.47-7.36 (m, 2H), 7.33 (d, J=4.4 Hz, 1H), 7.06-6.96 (m, 1H), 3.84 (s, 3H), 2.11 (s, 2H), 1.83 (s, 2H), 1.69 (s, 5H). [M+H] Calc'd for C₂₅H₂₃N₃O₃, 414. Found, 414.

### Example 2.10: 2-Cyclopentyl-4-(2-(3-ethoxyphenyl)pyrazolo[1,5-a]pyrimidin-7-yl)benzoic Acid

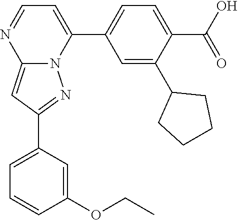 [SMILES: CCOc1cccc(-c2cc3nccc(-c4ccc(C(=O)O)c(C5CCCC5)c4)n3n2)c1]

The title compound was prepared in 21% yield according to the general procedure for the preparation of Example 2.2 using (3-ethoxyphenyl)boronic acid. ¹H NMR (400 MHz, DMSO-d₆) δ 13.24 (s, 1H), 8.64 (d, J=4.4 Hz, 1H), 8.52 (d, J=1.8 Hz, 1H), 8.00 (dd, J=8.1, 1.8 Hz, 1H), 7.86 (d, J=8.2 Hz, 1H), 7.68-7.57 (m, 2H), 7.46-7.32 (m, 3H), 7.05-6.95 (m, 1H), 4.11 (q, J=6.9 Hz, 2H), 3.87-3.76 (m, 1H), 2.12 (s, 2H), 1.85 (s, 2H), 1.71 (s, 5H), 1.38 (t, J=7.0 Hz, 3H). [M+H] Calc'd for C₂₆H₂₅N₃O₃, 428. Found, 428.

### Example 2.11: 2-Cyclopentyl-4-(2-(3-(cyclopropylmethoxy)phenyl)pyrazolo[1,5-a]pyrimidin-7-yl)benzoic Acid

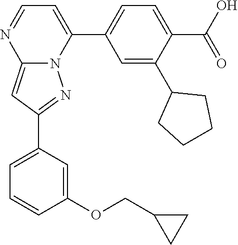 [SMILES: O=C(O)c1ccc(-c2ccnc3cc(-c4cccc(OCC5CC5)c4)nn23)cc1C1CCCC1]

### Preparation 2.11A: 1-Bromo-3-(cyclopropylmethoxy)benzene

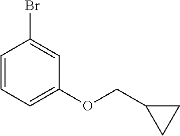 [SMILES: Brc1cccc(OCC2CC2)c1]

To a stirred solution of 3-bromophenol (1000 mg, 5.78 mmol) in DMF (20 mL) was added bromomethylcyclopropane (1171 mg, 8.67 mmol) and K₂CO₃ (1838 mg, 17.34 mmol). The resulting mixture was stirred at 80° C. for 2 hours and purified by flash column chromatography. Pure fractions were evaporated to dryness to afford 1-bromo-3-(cyclopropylmethoxy)benzene (1230 mg, 94%) as a yellow solid. ¹H NMR (300 MHz, DMSO-d₆) δ 7.30-7.18 (m, 1H), 7.17-7.06 (m, 2H), 6.95 (ddd, J=8.3, 2.3, 1.1 Hz, 1H), 3.84 (d, J=7.0 Hz, 2H), 1.31-1.12 (m, 1H), 0.64-0.50 (m, 2H), 0.43-0.27 (m, 2H). [M+H] Calc'd for C₁₀H₁₁BrO, 228. Found, 228.

### Preparation 2.11B: 2-(3-(Cyclopropylmethoxy)phenyl)-4,4,5,5-tetramethyl-1,3,2-dioxaborolane

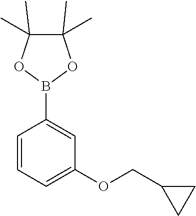 [SMILES: CC1(C)OB(c2cccc(OCC3CC3)c2)OC1(C)C]

To a stirred solution of 1-bromo-3-(cyclopropylmethoxy)benzene (1230 mg, 5.42 mmol) was added 4,4,5,5-tetramethyl-2-(4,4,5,5-tetramethyl-1,3,2-dioxaborolan-2-yl)-1,3,2-dioxaborolane (1650 mg, 6.50 mmol) in 1,4-dioxane (20 mL) was added Pd(dppf)Cl₂ (792 mg, 1.08 mmol) and saturated Na₂CO₃ solution (4 mL) under nitrogen. The resulting mixture was stirred at 80° C. for 2 hours and purified by flash column chromatography. Pure fractions were evaporated to dryness to afford 2-cyclopentyl-4-(5-fluoro-1H-pyrrolo[2,3-b]pyridin-4-yl)benzoic acid (530 mg, 36%) as a yellow solid. [M+H] Calc'd for C₁₆H₂₃BO₃, 275. Found, 275.

The title compound was prepared in 10% yield according to the general procedure for the preparation of Example 2.2 using 2-[3-(cyclopropylmethoxy)phenyl]-4,4,5,5-tetramethyl-1,3,2-dioxaborolane (Preparation 2.11B). ¹H NMR (400 MHz, DMSO-d₆) δ 13.24 (s, 1H), 8.63 (d, J=4.4 Hz, 1H), 8.51 (d, J=1.7 Hz, 1H), 7.99 (dd, J=8.2, 1.7 Hz, 1H), 7.86 (d, J=8.1 Hz, 1H), 7.67-7.57 (m, 2H), 7.45-7.31 (m, 3H), 6.99 (dd, J=8.2, 2.5 Hz, 1H), 3.90 (d, J=7.0 Hz, 2H), 3.81 (t, J=8.1 Hz, 1H), 2.11 (s, 2H), 1.85 (s, 2H), 1.70 (s, 5H), 0.60 (dt, J=8.0, 3.0 Hz, 2H), 0.41-0.30 (m, 2H). [M+H] Calc'd for C₂₈H₂₇N₃O₃, 454. Found, 454.

### Example 2.12: 2-Cyclopentyl-4-(2-(1-methyl-H-pyrazol-4-yl)pyrazolo[1,5-a]pyrimidin-7-yl)benzoic Acid

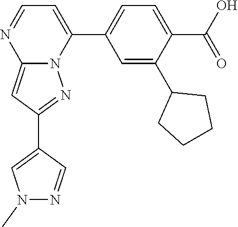 [SMILES: Cn1cc(-c2cc3nccc(-c4ccc(C(=O)O)c(C5CCCC5)c4)n3n2)cn1]

The title compound was prepared in 41% yield according to the general procedure for the preparation of Example 2.2 using 1-methyl-4-(4,4,5,5-tetramethyl-1,3,2-dioxaborolan-2-yl)pyrazole. ¹H NMR (400 MHz, DMSO-d₆) δ 8.55 (s, 1H), 8.28 (s, 1H), 8.19 (s, 1H), 7.93 (s, 1H), 7.76 (s, 1H), 7.74 (s, 1H), 7.23 (s, 1H), 6.99 (s, 1H), 3.89-3.79 (m, 3H), 3.78 (s, 1H), 2.32-2.08 (d, 2H), 2.06-1.81 (d, 2H), 1.69-1.67 (m, 4H). [M+H] Calc'd for C₂₂H₂₁N₅O₂, 388. Found, 388.

### Example 2.13: (S)-4-(2-(4-Fluorophenyl)pyrazolo[1,5-a]pyrimidin-7-yl)-N-(2-hydroxy-1-phenylethyl)-2-methoxybenzamide

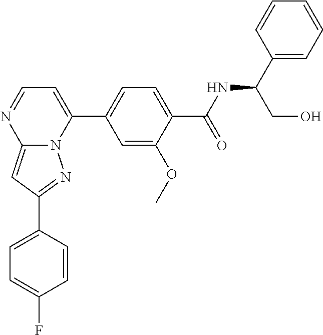 [SMILES: COc1cc(-c2ccnc3cc(-c4ccc(F)cc4)nn23)ccc1C(=O)N[C@H](CO)c1ccccc1]

### Preparation 2.13A: Methyl 4-(2-bromopyrazolo[1,5-a]pyrimidin-7-yl)-2-methoxybenzoate

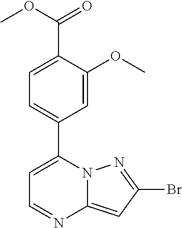 [SMILES: COC(=O)c1ccc(-c2ccnc3cc(Br)nn23)cc1OC]

To a stirred solution of methyl-2-methoxy-4-(4,4,5,5-tetramethyl-1,3,2-dioxaborolan-2-yl)benzoate (283 mg, 0.97 mmol) in 1,4-dioxane (10 mL) and water (2 mL) were added Na₂CO₃ (137 mg, 1.29 mmol), 7-bromo-2-chloro-pyrazolo[1,5-a]pyrimidine (150 mg, 0.65 mmol) and Pd(dppf)Cl₂ (94 mg, 0.13 mmol) at room temperature under N₂. The resulting mixture was heated to 100° C. for 2.5 h. LCMS showed completion of the reaction. The reaction mixture was diluted with ethyl acetate and washed with water; the organic layer was dried over Na₂SO₄, concentrated. The obtained yellow sticky solid was passed though flash column to obtain methyl 4-(2-bromopyrazolo[1,5-a]pyrimidin-7-yl)-2-methoxy-benzoate (100 mg, 43%). [M+H] Calc'd for C₁₅H₁₂BrN₃O₃, 362. Found, 362.

### Preparation 2.13B: Methyl 4-(2-(4-fluorophenyl)pyrazolo[1,5-a]pyrimidin-7-yl)-2-methoxybenzoate

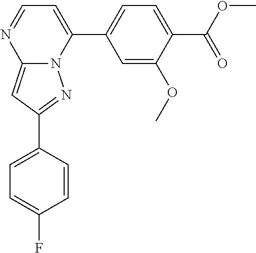 [SMILES: COC(=O)c1ccc(-c2ccnc3cc(-c4ccc(F)cc4)nn23)cc1OC]

To a stirred solution of methyl 4-(2-bromopyrazolo[1,5-a]pyrimidin-7-yl)-2-methoxy-benzoate (120 mg, 0.33 mmol) in 1,4-dioxane (10 mL) and water (2 mL) was added K₃PO₄ (217 mg, 0.99 mmol), Pd(dppf)Cl₂ (364 mg, 0.50 mmol) and (4-fluorophenyl) boronic acid (56 mg, 0.40 mmol) at room temperature under N₂. The resulting mixture was heated to 80° C. for 2.5 h. LCMS showed completion of the reaction. The reaction mixture was diluted with ethyl acetate and washed with water; the organic layer was dried over Na₂SO₄, concentrated. The obtained yellow sticky solid was passed though silica gel to obtain methyl 4-[2-(4-fluorophenyl)pyrazolo[1,5-a]pyrimidin-7-yl]-2-methoxy-benzoate (100 mg, 80%). [M+H] Calc'd for C₂₁H₁₆FN₃O₃, 378. Found, 378.

### Preparation 2.13C: 4-(2-(4-Fluorophenyl)pyrazolo[1,5-a]pyrimidin-7-yl)-2-methoxybenzoic Acid

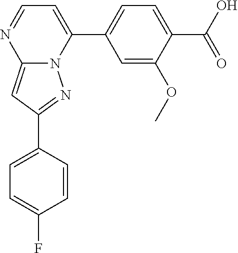 [SMILES: COc1cc(-c2ccnc3cc(-c4ccc(F)cc4)nn23)ccc1C(=O)O]

To a stirred solution of methyl 4-[2-(4-fluorophenyl)pyrazolo[1,5-a]pyrimidin-7-yl]-2-methoxy-benzoate (100 mg, 0.26 mmol) in methanol (9 mL) and water (1 mL) was added NaOH (106 mg, 2.65 mmol) at room temperature and the resulting mixture was warmed to 50° C. for 2.5 h. LCMS showed completion of the reaction. The reaction mixture was diluted with ethyl acetate and washed with water; the organic layer was dried over Na₂SO₄, concentrated. The obtained solid was purified by reverse phase column to obtain 4-[2-(4-fluorophenyl)pyrazolo[1,5-a]pyrimidin-7-yl]-2-methoxy-benzoic acid (70 mg, 73%). [M+H] Calc'd for C₂₀H₁₄FN₃O₃, 364. Found, 364.

To a stirred solution of 4-[2-(4-fluorophenyl)pyrazolo[1,5-a]pyrimidin-7-yl]-2-methoxy-benzoic acid (70 mg, 0.19 mmol, Preparation 2.13C) in DCM (10 mL) was added (2S)-2-amino-2-phenyl-ethanol (40 mg, 0.29 mmol), PyBop (150 mg, 0.29 mmol), and DIEA (0.07 mL, 0.39 mmol). The resulting mixture was stirred at room temperature for 2.5 h. LCMS showed the reaction was completed. The reaction mixture was diluted with ethyl acetate and washed with water; the organic layer was dried over Na₂SO₄, concentrated. The obtained solid was purified by reveres phase column and further purified by Prep-HPLC to obtain 4-[2-(4-fluorophenyl)pyrazolo[1,5-a]pyrimidin-7-yl]-N-[(1S)-2-hydroxy-1-phenyl-ethyl]-2-methoxy-benzamide (17 mg, 18%). ¹H NMR (400 MHz, DMSO-d₆) δ 8.78 (d, 1H), 8.64 (d, 1H), 8.11-8.13 (m, 3H), 7.85-7.92 (m, 2H), 6.97-7.42 (m, 10H), 5.07-5.11 (m, 1H), 3.92-4.05 (s, 3H), 3.66-3.75 (m, 2H). [M+H] Calc'd for C₂₈H₂₃FN₄O₃, 483. Found, 483.

### Example 3.1: 2-Cyclopentyl-4-[2-(4-fluorophenyl)pyrazolo[4,3-b]pyridin-7-yl]benzoic Acid

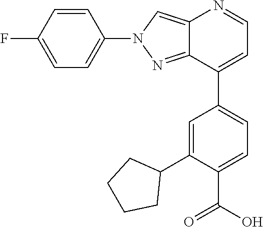 [SMILES: O=C(O)c1ccc(-c2ccnc3cn(-c4ccc(F)cc4)nc23)cc1C1CCCC1]

### Step 3.1A: 7-Iodo-1H-pyrazolo[4,3-b]pyridin-3-amine

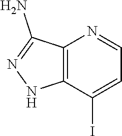 [SMILES: Nc1n[nH]c2c(I)ccnc12]

To a stirred solution of 3-fluoro-4-iodo-pyridine-2-carbonitrile (8.5 g, 34.27 mmol) in 1-butanol (200 mL) was added NH₂NH₂ (2 mL) at 0° C. The reaction was warmed to 105° C. for 4 h. Upon completion, the reaction mixture was concentrated in vacuo and the residue taken up in DCM. The organic layers were successively washed with brine, dried over anhydrous sodium sulfate, and concentrated in vacuo to obtain 7-iodo-1H-pyrazolo[4,3-b]pyridin-3-amine (10 g, >99% yield) as a brown solid. [M+H] Calc'd for C₆H₅IN₄, 261. Found, 261.

### Step 3.1B: 7-Iodo-1H-pyrazolo[4,3-b]pyridine-3-diazonium Acetate

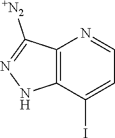

To a stirred solution of 7-iodo-2H-pyrazolo[4,3-b]pyridin-3-amine (10 g, 38 mmol) in acetic acid (3.2 mL, 38 mmol) and water (20 mL) was added a solution of NaNO₂ (5.3 g, 77 mmol) in water (20 mL) at 0° C. dropwise. The reaction was stirred at r.t. for 2 h. Upon completion the reaction mixture was filtered, and the filter cake was partially dried in vacuo to obtain crude 7-iodo-1H-pyrazolo[4,3-b]pyridine-3-diazonium acetate (10.3 g, 80.2% yield). [M+] Calc'd for C₆H₃IN₅⁺, 272. Found, 272.

### Step 3.1C: 7-Iodo-1H-pyrazolo[4,3-b]pyridine

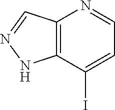

To a well-stirred solution of 7-iodo-1H-pyrazolo[4,3-b]pyridine-3-diazonium acetate (10 g, 30.1 mmol) in DME (300 mL)/water (250 mL) was added H₃PO₃ (25 g, 305 mmol) portion-wise at r.t. The resultant mixture was stirred at 45° C. for 3 h under N₂. Upon completion the pH was adjusted (˜9) by careful addition of sat. K₂CO₃ at 0° C. The mixture was extracted with ethyl acetate (3×), and the combined organic layers were successively washed with brine, dried over anhydrous sodium sulfate, filtered and concentrated in vacuo. The residue was purified by silica gel column chromatography (PE/EA, 10:1, 7:1 to 5:1) to obtain 7-iodo-1H-pyrazolo[4,3-b]pyridine (6 g, 81.3% yield) as a light-yellow solid. [M+H] Calc'd for C₆H₄IN₃, 246. Found, 246.

### Step 3.1D: 2-Cyclopentyl-4-(1H-pyrazolo[4,3-b]pyridin-7-yl)benzoate

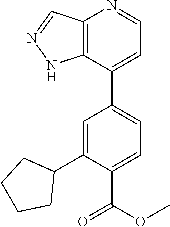 [SMILES: COC(=O)c1ccc(-c2ccnc3cn[nH]c23)cc1C1CCCC1]

To a stirred solution of (3-cyclopentyl-4-methoxycarbonyl-phenyl) boronic acid (364.5 mg, 1.47 mmol) in 1,4-dioxane (10 mL) were added 7-iodo-2H-pyrazolo[4,3-b]pyridine (300 mg, 1.2 mmol), saturated Na₂CO₃ (2 mL), and Pd(dppf)Cl₂ (20 mg, 0.03 mmol). The reaction mixture purged with N₂ and heated at 80° C. for 3 h. Upon completion, the reaction mixture was concentrated in vacuo, and the residue taken up in ethyl acetate. The organic layers were successively washed with brine, dried over anhydrous sodium sulfate, and concentrated in vacuo. The residue was purified by silica gel column chromatography (10-100% gradient of ethyl acetate in petroleum ether) to obtain methyl 2-cyclopentyl-4-(2H-pyrazolo[4,3-b]pyridin-7-yl)benzoate (215 mg, 54.6% yield). [M+H] Calc'd for C₁₉H₁₉N₃O₂, 322. Found, 322.

### Step 3.1E: 2-Cyclopentyl-4-[2-(4-fluorophenyl)pyrazolo[4,3-b]pyridin-7-yl]benzoate

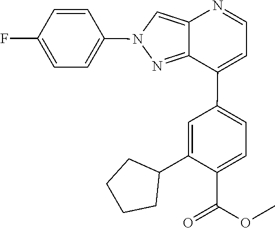 [SMILES: COC(=O)c1ccc(-c2ccnc3cn(-c4ccc(F)cc4)nc23)cc1C1CCCC1]

To a stirred solution of methyl 2-cyclopentyl-4-(1H-pyrazolo[4,3-b]pyridin-7-yl)benzoate (200 mg, 0.62 mmol) in DMF (3 mL) was added 1-fluoro-4-iodo-benzene (276 mg, 1.24 mmol), dimethylcyclohexane-1,2-diamine (55 mg, 0.24 mmol), K₃PO₄ (909 mg, 1.24 mmol) and CuI (23 mg, 0.12 mmol). The resulting mixture was purged with N₂ and heated at 150° C. for 2 h. Upon completion, the reaction mixture was diluted with ethyl acetate and washed with water. The combined organic layers were dried over sodium sulfate and concentrated in vacuo. The residue was purified by silica gel column chromatography (10-100% gradient of ethyl acetate in petroleum ether) to obtain methyl 2-cyclopentyl-4-[2-(4-fluorophenyl)pyrazolo[4,3-b]pyridin-7-yl]benzoate (80 mg, 30.9% yield). [M+H] Calc'd for C₂₅H₂₂FN₃O₂, 416. Found, 416.

### Step 3.1F: 2-Cyclopentyl-4-[2-(4-fluorophenyl)pyrazolo[4,3-b]pyridin-7-yl]benzoic Acid

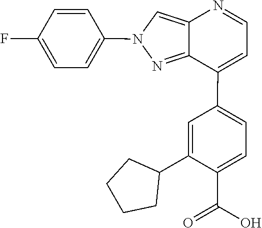 [SMILES: O=C(O)c1ccc(-c2ccnc3cn(-c4ccc(F)cc4)nc23)cc1C1CCCC1]

To a stirred solution of methyl 2-cyclopentyl-4-[2-(4-fluorophenyl)pyrazolo[4,3-b]pyridin-7-yl]benzoate (50 mg, 0.12 mmol) in THF (3 mL) and methanol (2 mL) was added a solution of NaOH (50 mg, 1.3 mmol) in water (2 mL) at r.t. The reaction was stirred at 50° C. for overnight. Upon completion, the reaction mixture was concentrated and pH adjusted to ˜3-4 with citric acid. Brine was added to the resulting mixture, and it was extracted with THF. The combined organic layers were dried over sodium sulfate and concentrated in vacuo. The residue was purified by Prep-HPLC to afford the title compound (13.0 mg, 25.7%) as a yellow solid. [M+H] Calc'd for C₂₄H₂₀FN₃O₂, 402. Found 402. ¹H NMR (400 MHz, DMSO-d₆) δ 9.57 (s, 1H), 8.70 (d, J=4.4 Hz, 1H), 8.56 (d, J=1.8 Hz, 1H), 8.29-8.20 (m, 2H), 8.14 (dd, J=8.2, 1.8 Hz, 1H), 7.84 (d, J=8.1 Hz, 1H), 7.75 (d, J=4.5 Hz, 1H), 7.58-7.47 (m, 2H), 3.86 (m, J=8.4 Hz, 1H), 2.18-2.08 (m, 2H), 1.87 (s, 2H), 1.72 (m, J=5.9, 5.1 Hz, 4H). Prep-HPLC conditions [Column: XSelect CSH Prep C18 OBD Column, Sum, 19*150 mm; Mobile Phase A: Water (0.05% TFA), Mobile Phase B: ACN; Flow rate: 25 mL/min; Gradient: 44% B to 66% B in 7 min; 220/254 nm; Rt: 6.18 min]

### Example 3.2: 2-Cyclopentyl-4-(2-phenyl-2H-pyrazolo[4,3-b]pyridin-7-yl)benzoic Acid

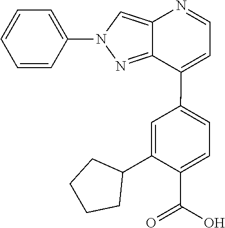 [SMILES: O=C(O)c1ccc(-c2ccnc3cn(-c4ccccc4)nc23)cc1C1CCCC1]

The title compound was prepared in 5.4% overall yield as an off-white solid according to the preparation of Example 3.1 using iodobenzene in STEP E. [M+H] Calc'd for C₂₄H₂₁N₃O₂, 384. Found 384. ¹H NMR (400 MHz, DMSO-d₆) δ 12.97 (s, 1H), 9.57 (s, 1H), 8.70 (d, 1H), 8.61 (d, 1H), 8.25-8.20 (m, 2H), 8.13 (dd, 1H), 7.84 (d, 1H), 7.73 (d, 1H), 7.70-7.63 (m, 2H), 7.57-7.50 (m, 1H), 3.87 (q, 1H), 2.12 (s, 2H), 1.88 (s, 2H), 1.73 (s, 4H).

### Example 3.3: 2-Cyclopentyl-4-(2-(m-tolyl)-2H-pyrazolo[4,3-b]pyridin-7-yl)benzoic Acid

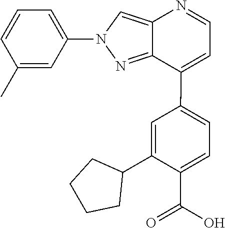 [SMILES: Cc1cccc(-n2cc3nccc(-c4ccc(C(=O)O)c(C5CCCC5)c4)c3n2)c1]

The title compound was prepared in 12.9% overall yield as a yellow solid according to the preparation of Example 3.1 using 3-methyl-iodobenzene in STEP E. [M+H] Calc'd for C₂₅H₂₃N₃O₂, 398. Found 398. ¹H NMR (400 MHz, DMSO-d₆) δ 9.55 (s, 1H), 8.67 (dd, J=6.7, 3.1 Hz, 2H), 8.09 (s, 1H), 8.08-7.98 (m, 2H), 7.74 (dd, J=17.5, 6.3 Hz, 2H), 7.53 (t, J=7.8 Hz, 1H), 7.34 (d, J=7.6 Hz, 1H), 3.91-3.86 (m, 1H), 2.47 (s, 3H), 2.2-2.08 (m, 2H), 1.91-1.85 (s, 2H), 1.80-1.69 (m, 4H).

### Example 3.4: 2-Cyclopentyl-4-(2-(3-cyclopropylphenyl)-2H-pyrazolo[4,3-b]pyridin-7-yl)benzoic Acid

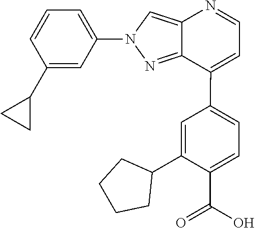 [SMILES: O=C(O)c1ccc(-c2ccnc3cn(-c4cccc(C5CC5)c4)nc23)cc1C1CCCC1]

The title compound was prepared in 13.7% overall yield as a yellow solid according to the preparation of Example 3.1 using 3-cyclopropylbenzene in STEP E. [M+H] Calc'd for C₂₇H₂₅N₃O₂, 424. Found 424. ¹H NMR (400 MHz, DMSO-d₆) δ 9.57 (s, 1H), 8.68 (s, 2H), 8.07 (s, 1H), 7.96 (d, J=8.1 Hz, 1H), 7.89 (t, J=2.0 Hz, 1H), 7.69 (d, J=4.3 Hz, 2H), 7.50 (t, J=7.9 Hz, 1H), 7.28-7.21 (m, 1H), 3.96 (s, 1H), 2.28-2.03 (m, 3H), 1.86 (br, 2H), 1.86-1.61 (m, 4H), 1.11-1.02 (m, 2H), 0.89-0.80 (m, 2H).

### Example 3.5: 2-Cyclopentyl-4-(6-fluoro-2-(4-fluorophenyl)-2H-pyrazolo[4,3-b]pyridin-7-yl)benzoic Acid

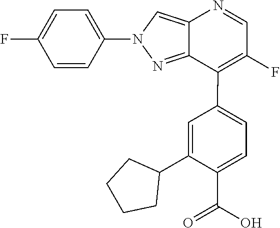 [SMILES: O=C(O)c1ccc(-c2c(F)cnc3cn(-c4ccc(F)cc4)nc23)cc1C1CCCC1]

The title compound was prepared in 14% overall yield as a white solid according to the preparation of Example 3.1 using 3,5-difluoro-4-iodopicolinonitrile in STEP A. [M+H] Calc'd for C₂₄H₁₉F₂N₃O₂, 420. Found 420. ¹H NMR (400 MHz, DMSO-d₆) δ 13.15 (s, 1H), 9.60 (s, 1H), 8.73 (d, 1H), 8.19-8.13 (m, 3H), 7.82-7.77 (m, 1H), 7.49 (t, 2H), 3.86 (m, 1H), 2.14-2.05 (m, 2H), 1.87-1.76 (m, 2H), 1.74-1.57 (m, 4H).

### Example 3.6: 4-(2-(3-Chlorophenyl)-2H-pyrazolo[4,3-b]pyridin-7-yl)-2-cyclopentylbenzoic Acid

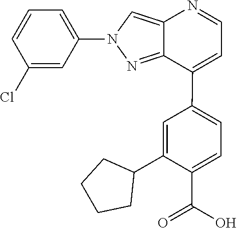 [SMILES: O=C(O)c1ccc(-c2ccnc3cn(-c4cccc(Cl)c4)nc23)cc1C1CCCC1]

The title compound was prepared in 6.1% overall yield as an off-white solid according to the preparation of Example 3.1 using 3-chloro-iodobenzene in STEP E. [M+H] Calc'd for C₂₄H₂₀ClN₃O₂, 418. Found 418. ¹H NMR (400 MHz, DMSO-d₆) δ 9.67 (s, 1H), 8.70 (dd, J=10.4, 3.1 Hz, 2H), 8.35 (t, J=2.1 Hz, 1H), 8.22 (dt, J=8.2, 1.4 Hz, 1H), 8.09 (dd, J=8.2, 1.8 Hz, 1H), 7.83 (d, J=8.2 Hz, 1H), 7.77 (d, J=4.5 Hz, 1H), 7.69 (t, J=8.1 Hz, 1H), 7.60 (dd, J=7.8, 2.1 Hz, 1H), 3.94-3.85 (m, 1H), 2.19-2.09 (m, 2H), 1.95-1.87 (s, 2H), 1.8-1.6 (m, 4H).

### Example 3.7: 4-(2-(3-Chloro-5-methylphenyl)-6-fluoro-2H-pyrazolo[4,3-b]pyridin-7-yl)-2-cyclopentylbenzoic Acid

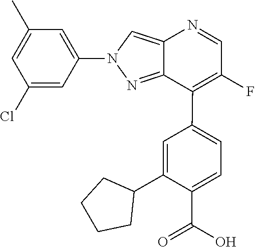 [SMILES: Cc1cc(Cl)cc(-n2cc3ncc(F)c(-c4ccc(C(=O)O)c(C5CCCC5)c4)c3n2)c1]

The title compound was prepared in 4.6% overall yield as a white solid according to the preparation of Example 3.1 using 3,5-difluoro-4-iodopicolinonitrile in STEP A and 3-chloro-5-methyl-iodobenzene in STEP E. [M+H] Calc'd for C₂₅H₂₁ClFN₃O₂, 450. Found 450. ¹H NMR (400 MHz, DMSO-d₆) δ 9.66 (s, 1H), 8.74 (d, J=3.0 Hz, 1H), 8.27 (s, 1H), 8.08 (s, 1H), 8.00 (s, 1H), 7.79 (q, J=8.2 Hz, 2H), 7.42 (s, 1H), 3.89 (m, 1H), 2.44 (s, 3H), 2.15-2.05 (m, 2H), 1.88-1.77 (m, 2H), 1.73-1.57 (m, 4H).

### Example 3.8: 2-Cyclopentyl-4-(6-fluoro-2-(3-methyl-5-(trifluoromethyl)phenyl)-2H-pyrazolo[4,3-b]pyridin-7-yl)benzoic Acid

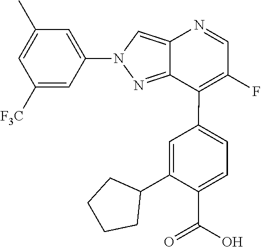 [SMILES: Cc1cc(-n2cc3ncc(F)c(-c4ccc(C(=O)O)c(C5CCCC5)c4)c3n2)cc(C(F)(F)F)c1]

The title compound was prepared in 15.2% overall yield as an off-white solid according to the preparation of Example 3.1 using 3,5-difluoro-4-iodopicolinonitrile in STEP A and 1-iodo-3-methyl-5-(trifluoromethyl)benzene in STEP E. [M+H] Calc'd for C₂₆H₂₁F₄N₃O₂, 484. Found 484. ¹H NMR (400 MHz, DMSO-d₆) δ 9.77 (s, 1H), 8.76 (d, J=3.1 Hz, 1H), 8.34 (d, J=10.2 Hz, 2H), 8.28 (s, 1H), 7.79 (d, J=7.2 Hz, 1H), 7.73 (d, J=13.0 Hz, 2H), 3.86 (s, 1H), 2.56 (s, 3H), 2.13-2.05 (s, 2H), 1.89-1.77 (m, 2H), 1.72-1.62 (m, 4H).

### Example 3.9: 2-Cyclopentyl-4-(6-fluoro-2-(3-(trifluoromethyl)phenyl)-2H-pyrazolo[4,3-b]pyridin-7-yl)benzoic Acid

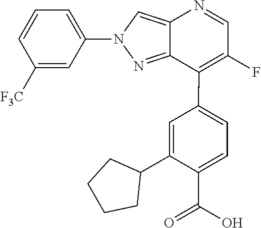 [SMILES: O=C(O)c1ccc(-c2c(F)cnc3cn(-c4cccc(C(F)(F)F)c4)nc23)cc1C1CCCC1]

The title compound was prepared in 5.9% overall yield as an off-white solid according to the preparation of Example 3.1 using 3,5-difluoro-4-iodopicolinonitrile in STEP A and 1-iodo-3-(trifluoromethyl)benzene in STEP E. [M+H] Calc'd for C₂₅H₁₉F₄N₃O₂, 470. Found 470. ¹H NMR (400 MHz, DMSO-d₆): δ 9.79 (s, 1H), 8.76 (d, J=3.0 Hz, 1H), 8.53-8.44 (m, 2H), 8.25 (s, 1H), 7.87 (d, J=5.0 Hz, 2H), 7.80 (d, J=7.9 Hz, 1H), 7.76 (d, J=8.1 Hz, 1H), 3.85 (t, J=8.3 Hz, 1H), 2.10 (dd, J=9.7, 5.3 Hz, 2H), 1.80 (m, 2H), 1.73-1.61 (m, 4H).

### Example 3.10: 4-(2-(3-Chloro-5-fluorophenyl)-6-fluoro-2H-pyrazolo[4,3-b]pyridin-7-yl)-2-cyclopentylbenzoic Acid

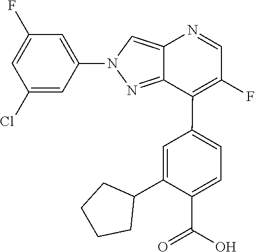 [SMILES: O=C(O)c1ccc(-c2c(F)cnc3cn(-c4cc(F)cc(Cl)c4)nc23)cc1C1CCCC1]

The title compound was prepared in 6.9% overall yield as a white solid according to the preparation of Example 3.1 using 3,5-difluoro-4-iodopicolinonitrile in STEP A and 1-chloro-3-fluoro-5-iodobenzene in STEP E. [M+H] Calc'd for C₂₄H₁₈F₂N₃O₂, 454. Found 454. ¹H NMR (400 MHz, DMSO-d₆) δ 13.18 (s, 1H), 9.73 (s, 1H), 8.77 (d, J=3.1 Hz, 1H), 8.28 (d, J=1.7 Hz, 1H), 8.16 (d, J=2.2 Hz, 1H), 8.11-8.07 (m, 1H), 7.89-7.78 (m, 2H), 7.64-7.61 (m, 1H), 3.85 (d, J=8.0 Hz, 1H), 2.16-2.08 (m, 2H), 1.87-1.79 (m, 2H), 1.72-1.65 (m, 4H).

### Example 3.11: 2-Cyclopentyl-4-(6-fluoro-2-(3-fluoro-5-methylphenyl)-2H-pyrazolo[4,3-b]pyridin-7-yl)benzoic Acid

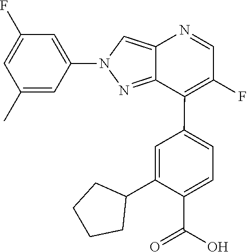 [SMILES: Cc1cc(F)cc(-n2cc3ncc(F)c(-c4ccc(C(=O)O)c(C5CCCC5)c4)c3n2)c1]

The title compound was prepared in 9.1% overall yield as an off-white solid according to the preparation of Example 3.1 using 3,5-difluoro-4-iodopicolinonitrile in STEP A and 1-fluoro-3-iodo-5-methylbenzene in STEP E. [M+H] Calc'd for C₂₅H₂₁F₂N₃O₂, 434; Found 434. ¹H NMR (400 MHz, DMSO-d₆) δ 13.12 (s, 1H), 9.63 (s, 1H), 8.74 (d, J=3.1 Hz, 1H), 8.28 (s, 1H), 7.84 (dt, J=11.8, 8.2 Hz, 4H), 7.19 (dd, J=9.5, 2.3 Hz, 1H), 3.86 (t, J=8.3 Hz, 1H), 2.44 (s, 3H), 2.17-2.06 (m, 2H), 1.87-1.78 (m, 2H), 1.75-1.67 (m, 4H).

### Example 3.12: 2-Cyclopentyl-4-(2-(3,5-difluorophenyl)-6-fluoro-2H-pyrazolo[4,3-b]pyridin-7-yl)benzoic Acid

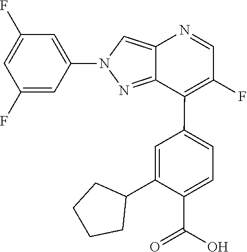 [SMILES: O=C(O)c1ccc(-c2c(F)cnc3cn(-c4cc(F)cc(F)c4)nc23)cc1C1CCCC1]

The title compound was prepared in 5.5% overall yield as a white solid according to the preparation of Example 3.1 using 3,5-difluoro-4-iodopicolinonitrile in STEP A and 1,3-difluoro-5-iodobenzene in STEP E. [M+H] Calc'd for C₂₄H₁₈F₃N₃O₂, 438. Found 438. ¹H NMR (400 MHz, DMSO-d₆) δ 13.18 (s, 1H), 9.72 (s, 1H), 8.77 (d, J=3.1 Hz, 1H), 8.27-8.22 (m, 1H), 8.02-7.91 (m, 2H), 7.86 (d, J=8.5 Hz, 1H), 7.81 (d, J=8.1 Hz, 1H), 7.47-7.42 (m, 1H), 3.87 (d, J=7.1 Hz, 1H), 2.13-2.07 (m, 2H), 1.86-1.79 (m, 2H), 1.71-1.64 (m, 4H).

### Example 3.13: 2-Cyclopentyl-4-(6-fluoro-2-(3-methoxy-5-(trifluoromethyl)phenyl)-2H-pyrazolo[4,3-b]pyridin-7-yl)benzoic Acid

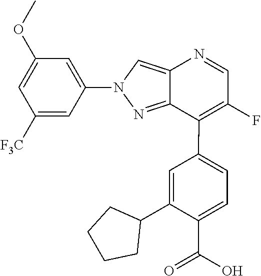 [SMILES: COc1cc(-n2cc3ncc(F)c(-c4ccc(C(=O)O)c(C5CCCC5)c4)c3n2)cc(C(F)(F)F)c1]

The title compound was prepared in 21.5% overall yield as a white solid according to the preparation of Example 3.1 using 3,5-difluoro-4-iodopicolinonitrile in STEP A and 1-iodo-3-methoxy-5-(trifluoromethyl)benzene in STEP E. [M+H] Calc'd for C₂₆H₂₁F₄N₃O₃, 500. Found 500. ¹H NMR (400 MHz, DMSO-d₆) δ 9.83 (s, 1H), 8.76 (d, J=3.1 Hz, 1H), 8.30 (s, 1H), 8.10 (s, 1H), 8.05 (d, J=2.4 Hz, 1H), 7.79 (s, 2H), 7.39 (s, 1H), 3.98 (s, 3H), 3.92-3.81 (m, 1H), 2.18-2.06 (m, 2H), 1.83-1.78 (m 2H), 1.69-1.64 (m, 4H).

### Example 3.14: 2-Cyclopentyl-4-(6-fluoro-2-(3-fluoro-5-(trifluoromethyl)phenyl)-2H-pyrazolo[4,3-b]pyridin-7-yl)benzoic Acid

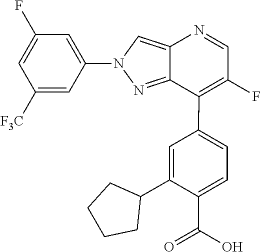 [SMILES: O=C(O)c1ccc(-c2c(F)cnc3cn(-c4cc(F)cc(C(F)(F)F)c4)nc23)cc1C1CCCC1]

The title compound was prepared in 24.9% overall yield as a white solid according to the preparation of Example 3.1 using 3,5-difluoro-4-iodopicolinonitrile in STEP A and 1-fluoro-3-iodo-5-(trifluoromethyl)benzene in STEP E. [M+H] Calc'd for C₂₅H₁₈F₅N₃O₂, 488. Found 488. ¹H NMR (400 MHz, DMSO-d₆) δ 9.82 (s, 1H), 8.77 (d, J=3.1 Hz, 1H), 8.47-8.37 (m, 2H), 8.26 (d, J=1.7 Hz, 1H), 7.88 (d, J=8.3 Hz, 1H), 7.78 (q, J=8.2 Hz, 2H), 3.87-3.82 (m, 1H), 2.12-2.08 (m, 2H), 1.83-1.78 (m, 2H), 1.69-1.64 (m, 4H).

### Example 3.15: 4-(2-(3-Chloro-5-(trifluoromethyl)phenyl)-6-fluoro-2H-pyrazolo[4,3-b]pyridin-7-yl)-2-cyclopentylbenzoic

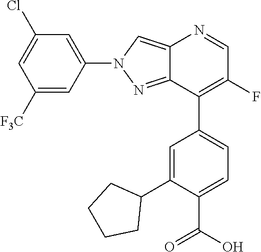 [SMILES: O=C(O)c1ccc(-c2c(F)cnc3cn(-c4cc(Cl)cc(C(F)(F)F)c4)nc23)cc1C1CCCC1]

The title compound was prepared in 19% overall yield as a white solid according to the preparation of Example 3.1 using 3,5-difluoro-4-iodopicolinonitrile in STEP A and 1-chloro-3-iodo-5-(trifluoromethyl)benzene in STEP E. [M+H] Calc'd for C₂₅H₁₈ClF₄N₃O₂, 504. Found 504. ¹H NMR (400 MHz, DMSO-d₆) δ 9.85 (s, 1H), 8.78 (d, J=3.0 Hz, 1H), 8.63 (s, 1H), 8.51 (s, 1H), 8.29 (s, 1H), 8.04 (s, 1H), 7.79 (s, 2H), 3.90-3.88 (m, 1H), 2.14-2.09 (m, 2H), 1.85-1.80 (m, 2H), 1.70-1.66 (s, 4H).

### Example 3.16: 2-Cyclopentyl-4-(2-(3,5-dichlorophenyl)-6-fluoro-2H-pyrazolo[4,3-b]pyridin-7-yl)benzoic Acid

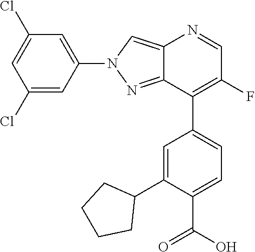 [SMILES: O=C(O)c1ccc(-c2c(F)cnc3cn(-c4cc(Cl)cc(Cl)c4)nc23)cc1C1CCCC1]

The title compound was prepared in 3.9% overall yield as a white solid according to the preparation of Example 3.1 using 3,5-difluoro-4-iodopicolinonitrile in STEP A and 1,3-dichloro-5-iodobenzene in STEP E. [M+H] Calc'd for C₂₄H₁₈Cl₂FN₃O₂, 470. Found 470. ¹H NMR (400 MHz, DMSO-d₆) δ 9.75 (s, 1H), 8.76 (d, J=3.1 Hz, 1H), 8.28-8.27 (m, 3H), 7.85-7.74 (m, 3H), 3.88 (s, 1H), 2.10 (q, J=8.5, 7.3 Hz, 2H), 1.88-1.81 (m, 2H), 1.72-1.65 (m, 4H).

### Example 3.17: 2-Cyclopentyl-4-(6-fluoro-2-(3-fluoro-5-methoxyphenyl)-2H-pyrazolo[4,3-b]pyridin-7-yl)benzoic Acid

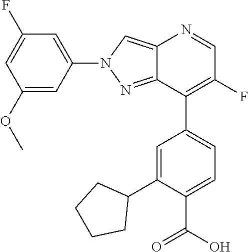 [SMILES: COc1cc(F)cc(-n2cc3ncc(F)c(-c4ccc(C(=O)O)c(C5CCCC5)c4)c3n2)c1]

The title compound was prepared in 23.6% overall yield as a white solid according to the preparation of Example 3.1 using 3,5-difluoro-4-iodopicolinonitrile in STEP A and 1-fluoro-3-iodo-5-methoxybenzene in STEP E. [M+H] Calc'd for C₂₅H₂₁F₂N₃O₃, 500. Found 500. ¹H NMR (400 MHz, DMSO-d₆) δ 13.27 (s, 1H), 9.70 (s, 1H), 8.75 (d, J=3.1 Hz, 1H), 8.25 (d, J=1.6 Hz, 1H), 7.86-7.75 (m, 2H), 7.64 (dd, J=9.6, 2.2 Hz, 2H), 7.02-6.98 (m, 1H), 3.89 (s, 3H), 2.12-2.07 (m, 2H), 1.85-1.76 (m, 2H), 1.70-1.63 (m, 4H).

### Example 3.18: 2-Cyclopentyl-4-(6-fluoro-2-(3-methyl-5-(trifluoromethoxy)phenyl)-2H-pyrazolo[4,3-b]pyridin-7-yl)benzoic Acid

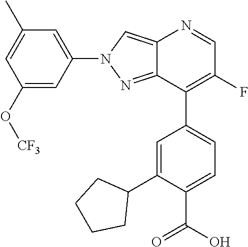 [SMILES: Cc1cc(OC(F)(F)F)cc(-n2cc3ncc(F)c(-c4ccc(C(=O)O)c(C5CCCC5)c4)c3n2)c1]

The title compound was prepared in 5% overall yield as an off-white solid according to the preparation of Example 3.1 using 3,5-difluoro-4-iodopicolinonitrile in STEP A and 1-iodo-3-methyl-5-(trifluoromethoxy)benzene in STEP E. [M+H] Calc'd for C₂₆H₂₁F₄N₃O₃, 500. Found 500. ¹H NMR (400 MHz, DMSO-d₆) δ 9.70 (s, 1H), 8.75 (d, J=3.0 Hz, 1H), 8.26 (s, 1H), 8.10 (s, 1H), 7.98 (s, 1H), 7.91-7.7 (m, 2H), 7.37 (s, 1H), 3.89-3.81 (m, 1H), 2.49 (s, 3H), 2.14-2.04 (m, 2H), 1.84-1.76 (m, 2H), 1.72-1.63 (m, 4H).

### Example 3.19: (S)-4-(2-(4-fluorophenyl)-2H-pyrazolo[4,3-b]pyridin-7-yl)-N-(2-hydroxy-1-phenylethyl)-2-methoxybenzamide

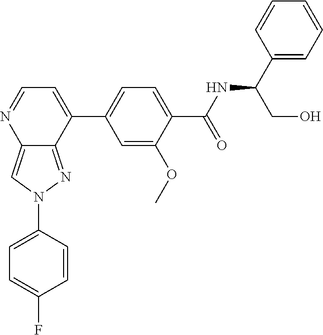 [SMILES: COc1cc(-c2ccnc3cn(-c4ccc(F)cc4)nc23)ccc1C(=O)N[C@H](CO)c1ccccc1]

### Preparation 3.19A: Methyl 2-methoxy-4-(2H-pyrazolo[4,3-b]pyridin-7-yl)benzoate

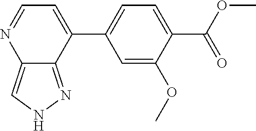 [SMILES: COC(=O)c1ccc(-c2ccnc3c[nH]nc23)cc1OC]

To a 40 mL sealed tube was placed 7-iodo-2H-pyrazolo[4,3-b]pyridine (200 mg, 0.82 mmol), methyl 2-methoxy-4-(4,4,5,5-tetramethyl-1,3,2-dioxaborolan-2-yl)benzoate (286 mg, 0.98 mmol), Pd(dppf)Cl₂ (119 mg, 0.16 mmol), Na₂CO₃ (260 mg, 2.45 mmol), water (2 mL) and 1,4-dioxane (10 mL) under N₂ atmosphere. The resulting mixture was stirred 3 h at 80° C. The LCMS showed completion of the reaction The reaction mixture was diluted with water, extracted with ethyl acetate and washed with water, the organic layer was dried over sodium sulphate, concentrated. The obtained crude product was passed though silica gel to obtain 160 mg of methyl 2-methoxy-4-(2H-pyrazolo[4,3-b]pyridin-7-yl)benzoate as an off-white solid. [M+H] Calc'd for C₁₅H₁₃N₃O₃, 284. Found, 284.

### Preparation 3.19B: Methyl 4-(2-(4-fluorophenyl)-2H-pyrazolo[4,3-b]pyridin-7-yl)-2-methoxybenzoate

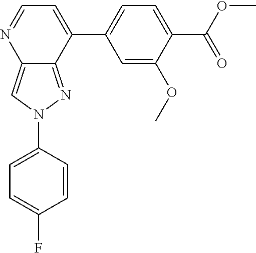 [SMILES: COC(=O)c1ccc(-c2ccnc3cn(-c4ccc(F)cc4)nc23)cc1OC]

To a 40 mL sealed tube was placed methyl 2-methoxy-4-(2H-pyrazolo[4,3-b]pyridin-7-yl)benzoate (150 mg, 0.53 mmol), 1-fluoro-4-iodo-benzene (141 mg, 0.64 mmol), rac-(1R,2R)—N¹,N²-dimethylcyclohexane-1,2-diamine (75 mg, 0.53 mmol), CuI (20 mg, 0.11 mmol), K₃PO₄ (225 mg, 1.06 mmol) and 1,4-dioxane (10 mL) under a nitrogen atmosphere. The resulting mixture was stirred overnight at 110° C. LCMS showed completion of the reaction. The organic layer was dried over sodium sulphate, concentrated. The obtained crude product was passed though silica gel to obtain 130 mg of methyl 4-[2-(4-fluorophenyl)pyrazolo[4,3-b]pyridin-7-yl]-2-methoxy-benzoate as an off-white solid. [M+H] Calc'd for C₂₁H₁₆FN₃O₃, 378. Found, 378.

### Preparation 3.19C: 4-(2-(4-Fluorophenyl)-2H-pyrazolo[4,3-b]pyridin-7-yl)-2-methoxybenzoic Acid

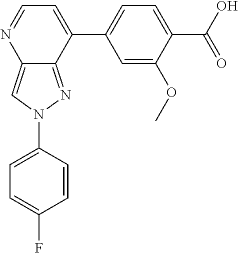 [SMILES: COc1cc(-c2ccnc3cn(-c4ccc(F)cc4)nc23)ccc1C(=O)O]

To a 100 mL round-bottom flask was placed methyl 4-[2-(4-fluorophenyl)pyrazolo[4,3-b]pyridin-7-yl]-2-methoxy-benzoate (150 mg, 0.40 mmol), NaOH (79 mg, 1.99 mmol), H₂O (5 mL), THF (5 mL), and methanol (5 mL). The resulting mixture was stirred for 3 h at 50° C. LCMS showed completion of the reaction. The pH value of the solution was adjusted to 3 with HCl (1 mol/L) and extracted with 50 mL DCM. The organic layers was combined and dried (Na₂SO₄) before concentration to dryness to obtain 110 mg of 4-[2-(4-fluorophenyl)pyrazolo[4,3-b]pyridin-7-yl]-2-methoxy-benzoic acid as an off-white solid. [M+H] Calc'd for C₂₀H₁₄FN₃O₃, 364. Found, 364.

To a 40 mL sealed tube was placed 4-[2-(4-fluorophenyl)pyrazolo[4,3-b]pyridin-7-yl]-2-methoxy-benzoic acid (110 mg, 0.30 mmol), rac-(2S)-2-amino-2-phenyl-ethanol (62 mg, 0.45 mmol), PyBOP (189 mg, 0.36 mmol), DIEA (59 mg, 0.45 mmol), and DCM (10 mL). The resulting mixture was stirred overnight at room temperature. The LCMS showed completion of the reaction. The organic layer was dried over sodium sulphate, concentrated. The obtained crude product was further purified by Prep-HPLC to afford 4-[2-(4-fluorophenyl)pyrazolo[4,3-b]pyridin-7-yl]-2-methoxy-N-[rac-(1S)-2-hydroxy-1-phenyl-ethyl]benzamide as a yellow solid (86 mg, 58.3%). ¹H-NMR (400 MHz, DMSO-d₆) δ 9.38 (s, 1H), 8.92 (s, 1H), 8.38 (s, 1H), 8.25-8.21 (d, 2H), 8.16-8.08 (m, 3H), 7.46-7.41 (m, 4H), 7.39-7.30 (m, 3H), 7.28 (s, 1H), 5.24 (s, 1H), 4.22-4.20 (m, 3H), 3.97-3.94 (d, 2H). [M+H] Calc'd for C₂₈H₂₃FN₄O₃, 483. Found, 483.

### Example 3.20: N-Cyano-2-cyclopentyl-4-(2-(4-fluorophenyl)-2H-pyrazolo[4,3-b]pyridin-7-yl)benzamide

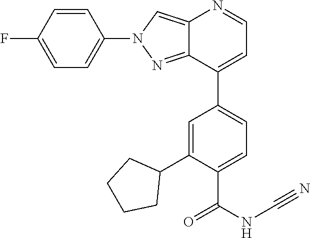 [SMILES: N#CNC(=O)c1ccc(-c2ccnc3cn(-c4ccc(F)cc4)nc23)cc1C1CCCC1]

To a stirred solution of 2-cyclopentyl-4-[2-(4-fluorophenyl)pyrazolo[4,3-b]pyridin-7-yl]benzoic acid (70 mg, 0.17 mmol) in DCM (10 mL) was added cyanamide (15 mg, 0.35 mmol), DCC (54 mg, 0.26 mmol), and DMAP (32 mg, 0.26 mmol). The reaction was stirred at 50° C. for 2 h. Upon completion, the reaction mixture was taken up in DCM and washed with water. The combined organic layers were dried over sodium sulfate and concentrated in vacuo. The residue was purified by Prep HPLC to furnish the title compound (58.8 mg, 78.9%) as a yellow solid. [M+H] Calc.'d for C₂₅H₂₀FN₅O, 426. Found 426. ¹H NMR (400 MHz, DMSO-d₆) δ 9.57 (s, 1H), 8.71 (d, J=4.4 Hz, 1H), 8.54 (d, J=1.7 Hz, 1H), 8.22 (m, J=8.2, 5.5, 3.2 Hz, 3H), 7.77 (d, J=4.4 Hz, 1H), 7.70 (d, J=8.1 Hz, 1H), 7.58-7.48 (m, 2H), 3.41 (s, 1H), 2.17-2.06 (m, 2H), 1.90-1.82 (m, 2H), 1.80-1.66 (m, 4H). Prep HPLC conditions [Column: Xselect CSH OBD Column 30*150 mm 5 um n; Mobile Phase A: Water (0.05% TFA), Mobile Phase B: ACN; Flow rate: 60 mL/min; Gradient: 20% B to 60% B in 7 min; 254/210 nm; Rt: 6.95 min].

### Example 3.21: 2-(Cyclopropylamino)-4-(6-fluoro-2-(4-fluorophenyl)-2H-pyrazolo[4,3-b]pyridin-7-yl)benzoic Acid

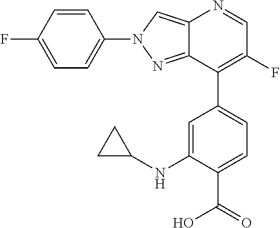 [SMILES: O=C(O)c1ccc(-c2c(F)cnc3cn(-c4ccc(F)cc4)nc23)cc1NC1CC1]

STEP 3.21A. A solution of methyl 4-bromo-2-fluoro-benzoate (1 g, 4.3 mmol), cyclopropanamine (490 mg, 8.6 mmol) and DIEA (2.24 mL, 12.9 mmol) in NMP (10 mL) was stirred at 100° C. for 2 h. Upon completion the reaction was diluted with brine and extracted with ethyl acetate (3×). The combined organic layers were successively dried over anhydrous sodium sulfate, filtered, and concentrated in vacuo. The residue was purified by a silica gel column chromatography, using PE as eluent, to obtain the crude product 4-bromo-2-(cyclopropylamino)benzoate (1.2 g) as a yellow solid. [M+H] Calc.'d for C₁₁H₁₂BrNO₂, 270. Found 270.

STEP 3.21B. A solution of methyl 4-bromo-2-(cyclopropylamino)benzoate (1.8 g, 6.7 mmol), 4,4,5,5-tetramethyl-2-(4,4,5,5-tetramethyl-1,3,2-dioxaborolan-2-yl)-1,3,2-dioxaborolane (3.4 g, 13.3 mmol), potassium acetate (1.68 g, 20.0 mmol) and Pd(dppf)Cl₂ (488 mg, 0.67 mmol) in 1,4-dioxane (30 mL) was purged with N₂ and stirred at 100° C. for 3 h. The reaction mixture was taken up in chloroform and washed with water. The organic layers were successively dried over sodium sulfate, filtered, and concentrated in vacuo. The residue was passed through a silica gel, and the crude product was used in the next step without further purification (2.4 g). [M+H] Calc.'d for C₁₇H₂₄BNO₄, 318. Found 318.

STEP 3.21C. A solution of 6-fluoro-7-iodo-2H-pyrazolo[4,3-b]pyridine (300 mg, 1.14 mmol), methyl-2-(cyclopropylamino)-4-(4,4,5,5-tetramethyl-1,3,2-dioxaborolan-2-yl)benzoate (543 mg, 1.71 mmol), Na₂CO₃ (sat.) (1 mL, 1.14 mmol) and Pd(dppf)Cl₂ (83.5 mg, 0.11 mmol) in 1,4-dioxane (5 mL) was purged with N₂ and stirred at 80° C. for 3 h. Upon completion the reaction was concentrated in vacuo and then taken up in water (10 mL) and extracted with ethyl acetate (3×). The combined organic layers were washed with brine, dried over anhydrous sodium sulfate, and concentrated in vacuo. The solid was purified by Prep-TLC to obtain methyl 2-(cyclopropylamino)-4-(6-fluoro-2H-pyrazolo[4,3-b]pyridin-7-yl)benzoate (130 mg). [M+H] Calc.'d for C₁₇H₁₅FN₄O₂, 327. Found 327.

STEP 3.21D. A mixture of methyl 2-(cyclopropylamino)-4-(6-fluoro-2H-pyrazolo[4,3-b]pyridin-7-yl)benzoate (100 mg, 0.31 mmol), 1-fluoro-4-iodo-benzene (136 mg, 0.61 mmol), N1,N2-dimethylcyclohexane-1,2-diamine (17.4 mg, 0.12 mmol), CuI (11.64 mg, 0.06 mmol) and K₃PO₄ (130 mg, 0.61 mmol) in 1,4-dioxane (6 mL) was purged with N₂ and stirred at 100° C. for 8 h. Upon completion the reaction was concentrated in vacuo. The residue was taken up in water and extracted with ethyl acetate (3×). The combined organic layers were concentrated in vacuo and purified by Prep-TLC to obtain the product (80 mg) as an off-white solid. [M+H] Calc.'d for C₂₃H₁₈F₂N₄O₂, 421. Found 421.

STEP 3.21E. A solution of methyl 2-(cyclopropylamino)-4-[6-fluoro-2-(4-fluorophenyl)pyrazolo[4,3-b]pyridin-7-yl]benzoate (50 mg, 0.12 mmol) and NaOH (24 mg, 0.6 mmol) with water (1 mL)/THF (2 mL)/methanol (2 mL) was stirred at 50° C. for 2 h. Upon completion the reaction was concentrated in vacuo. The residue taken up in brine, the pH adjusted to 5-6, and extracted with THF (3×). The combined organic layers were dried over anhydrous sodium sulfate, filtered, and concentrated in vacuo. The residue was purified by Prep-TLC and further purified by Prep-HPLC to afford the title compound (19.2 mg, 38.9% yield) as a yellow solid. [M+H] Calc.'d for C₂₂H₁₆F₂N₄O₂, 407. Found 407. ¹H NMR (400 MHz, DMSO-d₆) δ 12.86 (s, 1H), 9.61 (s, 1H), 8.74 (d, J=3.0 Hz, 1H), 8.24-8.14 (m, 2H), 8.02 (s, 1H), 7.96 (d, J=8.3 Hz, 1H), 7.86 (d, J=1.7 Hz, 1H), 7.54-7.41 (m, 2H), 7.22 (m, J=8.3, 1.8 Hz, 1H), 0.79 (m, J=6.6, 3.3 Hz, 2H), 0.61-0.53 (m, 2H). Prep-HPLC conditions [Column: Xselect CSH OBD Column 30*150 mm 5 um n; Mobile Phase A: Water (0.05% TFA), Mobile Phase B: ACN; Flow rate: 60 mL/min; Gradient: 50% B to 85% B in 7 min; 254/210 nm; Rt: 6.95 min].

### Example 4.1: 2-Cyclopentyl-4-(6-phenylpyrrolo[1,2-b]pyridazin-4-yl)benzoic Acid

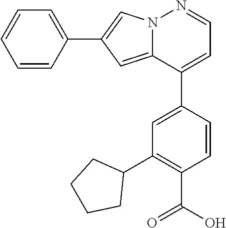 [SMILES: O=C(O)c1ccc(-c2ccnn3cc(-c4ccccc4)cc23)cc1C1CCCC1]

### Preparation 4.1A: 1-(4-Bromo-1H-pyrrol-2-yl)ethanone

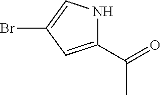

To a stirred solution of 1-(1H-pyrrol-2-yl)ethanone (3 .g, 27.5 mmol) in THF (20 mL) were added NBS (4.9 g, 27.5 mmol) and Amberlyst 15 (3.6 mg, 27.49 mmol). The reaction mixture was stirred at −40° C. for 1 h. LCMS showed the reaction completed. The reaction mixture was filtered, and the filtrate was quenched with saturated aq. Na₂SO₃. The residue was extracted with DCM and washed with sat. NaHCO₃. The organic layer was concentrated to obtain 1-(4-bromo-1H-pyrrol-2-yl)ethanone (4.7 g, 91% yield). MS: [M+H] Calc'd for C₆H₆BrNO, 188. Found 188.

### Preparation 4.1B: (E)-1-(4-Bromo-1H-pyrrol-2-yl)-3-(dimethylamino)prop-2-en-1-one

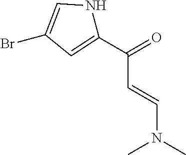

To a stirred solution of 1-(4-bromo-1H-pyrrol-2-yl)ethanone (2 g, 10.6 mmol) in THF was added DMF-DMA (20 mL, 10.6 mmol) at r.t. under N₂. The resulting solution was stirred at 80° C. for 1 h. LCMS showed the reaction complete. The reaction mixture was filtered, and the filter cake was washed with PE to obtain (E)-1-(4-bromo-1H-pyrrol-2-yl)-3-(dimethylamino)prop-2-en-1-one (800 mg, 31% yield) as a brown solid. MS: [M+H] Calc'd for C₉H₁₁BrN₂O, 243. Found 243.

### Preparation 4.1C: 6-Bromopyrrolo[1,2-b]pyridazin-4-ol

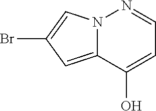 [SMILES: Oc1ccnn2cc(Br)cc12]

To a stirred solution of (E)-1-(4-bromo-1H-pyrrol-2-yl)-3-(dimethylamino)prop-2-en-1-one (400 mg, 1.65 mmol) in NMP (12 mL) was added amino 4-nitrobenzoate (460 mg, 2.53 mmol) and t-BuOK (280 mg, 2.5 mmol) at 0° C. under N₂. The resulting solution was stirred at r.t. for 1 h. The reaction mixture was adjusted to pH=3 with saturated citric acid solution and extracted with EA. The organic layer was dried over sodium sulphate and concentrated. The resulting orange sticky solid was purified by Prep-TLC to obtain 6-bromopyrrolo[1,2-b]pyridazin-4-ol (130 mg, 31% yield). MS: [M+H] Calc'd for C₇H₅BrN₂O, 213. Found 213.

### Preparation 4.1D: 6-Phenylpyrrolo[1,2-b]pyridazin-4-ol

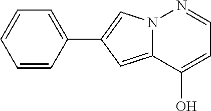 [SMILES: Oc1ccnn2cc(-c3ccccc3)cc12]

To a stirred solution of 6-bromopyrrolo[1,2-b]pyridazin-4-ol (100 mg, 0.47 mmol) in 1,4-dioxane (6 mL) were added a saturated solution of Na₂CO₃ (0.6 mL) and phenylboronic acid (114.5 mg, 0.94 mmol) at r.t. The resulting mixture was degassed by 3 cycles of vacuum/nitrogen. Pd(dppf)Cl₂ (8.9 mg, 0.010 mmol) was then added to above mixture under a nitrogen atmosphere. The solution was stirred at 100° C. for 2 h. LCMS showed the reaction completed. The reaction mixture was concentrated, and then diluted with EA and water. The value of PH of the solution was adjusted to 4-5 with a solution of HCl (2 mol/L). The resulting mixture was extracted with EA, and sequentially washed with water and saturated NaCl solution. The combined organic layers were dried over sodium sulphate and concentrated. The residue was purified by Prep-TLC to obtain 6-phenylpyrrolo[1,2-b]pyridazin-4-ol (110 mg, quantitative yield). MS: [M+H] Calc'd for C₁₃H₁₀N₂O, 211. Found 211.

### Preparation 4.1E: (6-Phenylpyrrolo[1,2-b]pyridazin-4-yl) trifluoromethanesulfonate

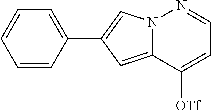 [SMILES: O=S(=O)(Oc1ccnn2cc(-c3ccccc3)cc12)C(F)(F)F]

To a stirred solution of 6-phenylpyrrolo[1,2-b]pyridazin-4-ol (110 mg, 0.52 mmol) in DCM (6 mL) was added TEA (0.08 mL, 0.61 mmol) at 0° C. A solution of Tf₂O (175 mg, 0.62 mmol) in DCM (0.5 mL) was added dropwise to above mixture at 0° C. under N₂. The solution was stirred at 0° C. for 1 h. LCMS showed the reaction completed. The reaction mixture was quenched with H₂O at 0° C., extracted with EA, and sequentially washed with water and saturated NaCl solution. The combined organic layers were dried over sodium sulphate and concentrated. The resulting orange sticky solid was purified by Prep-TLC to obtain (6-phenylpyrrolo[1,2-b]pyridazin-4-yl) trifluoromethanesulfonate (50 mg, 28% yield). [M+H] Calc'd for C₁₄H₉F₃N₂O₃S, 343. Found 343.

To a stirred solution of (6-phenylpyrrolo[1,2-b]pyridazin-4-yl) trifluoromethanesulfonate (50 mg, 0.15 mmol) in 1,4-dioxane (1 mL) were added 2-cyclopentyl-4-(4,4,5,5-tetramethyl-1,3,2-dioxaborolan-2-yl)benzoic acid (55.4 mg, 0.18 mmol), and sat. Na₂CO₃ (0.2 mL). The resulting mixture was degassed by 3 cycles of vacuum/nitrogen. Pd(dppf)₂CH₂Cl₂ (12 mg, 0.015 mmol) was then added to above mixture under a nitrogen atmosphere. The resulting mixture was stirred at 90° C. for 3 h. LCMS showed the reaction completed. The reaction mixture was concentrated and then diluted with ethyl acetate and water, and the value of PH of the solution was adjusted to 4-5 with a solution of HCl (2 mol/L). The resulting mixture was sequentially washed with water and saturated NaCl solution, and the organic layer was dried over sodium sulphate and concentrated. The residue was purified by Prep-TLC to obtain the crude product, and further purified by Prep-HPLC (Column: Sunfire Prep C18 OBD Column, 10 um, 19*250 mm; Mobile Phase A: Water (0.05% TFA), Mobile Phase B: ACN; Flow rate: 25 mL/min; Gradient: 75% B to 95% B in 8 min; 254/210 nm; Rt: 7.95 min) to obtain 2-cyclopentyl-4-(6-phenylpyrrolo[1,2-b]pyridazin-4-yl)benzoic acid (19.5 mg, 35% yield) as a yellow solid. MS: [M+H] Calc'd for C₂₅H₂₂N₂O₂, 383. Found 383. ¹H NMR (400 MHz, DMSO-d₆) δ 13.11 (s, 1H), 8.51 (d, J=1.8 Hz, 1H), 8.27 (d, J=4.7 Hz, 1H), 7.87-7.78 (m, 4H), 7.74 (dd, J=8.0, 1.8 Hz, 1H), 7.41 (t, J=7.7 Hz, 2H), 7.32-7.23 (m, 1H), 7.01 (d, J=1.8 Hz, 1H), 6.85 (d, J=4.7 Hz, 1H), 3.79 (q, J=8.4 Hz, 1H), 2.09 (s, 2H), 1.84-1.75 (m, 2H), 1.73-1.59 (m, 4H).

### Example 4.2: 2-Cyclopentyl-4-[6-(m-tolyl)pyrrolo[1,2-b]pyridazin-4-yl]benzoic Acid

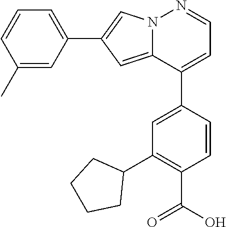 [SMILES: Cc1cccc(-c2cc3c(-c4ccc(C(=O)O)c(C5CCCC5)c4)ccnn3c2)c1]

The title compound was prepared from 6-bromopyrrolo[1,2-b]pyridazin-4-ol in 6.2% overall yield as a yellow solid according to the preparation of Example 4.1 by substituting m-tolylboronic acid in PREPARATION 35D. MS: [M+H] Calc'd for C₂₆H₂₄N₂O₂, 362. Found 362. ¹H NMR (DMSO-d₆, 400 MHz) δ 13.10 (s, 1H), 8.48 (s, 1H), 8.27 (d, J=4.7 Hz, 1H), 7.88-7.72 (m, 3H), 7.65 (s, 1H), 7.61 (d, J=7.7 Hz, 1H), 7.30 (t, J=7.6 Hz, 1H), 7.10 (d, J=7.5 Hz, 1H), 7.00 (s, 1H), 6.85 (d, J=4.7, 1H), 3.86-3.75 (m, 1H), 2.36 (s, 3H), 2.15-2.04 (m, 2H), 1.87-1.77 (m, 2H), 1.75-1.62 (m, 4H).

### Example 4.3: 2-Cyclopentyl-4-[6-(2-fluorophenyl)pyrrolo[1,2-b]pyridazin-4-yl]benzoic Acid

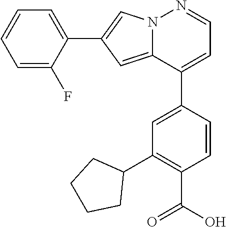 [SMILES: O=C(O)c1ccc(-c2ccnn3cc(-c4ccccc4F)cc23)cc1C1CCCC1]

### Preparation 4.3A: 6-(2-Fluorophenyl)pyrrolo[1,2-b]pyridazin-4-ol

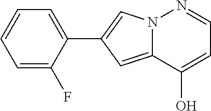 [SMILES: Oc1ccnn2cc(-c3ccccc3F)cc12]

To a stirred solution of 6-bromopyrrolo[1,2-b]pyridazin-4-ol (200 mg, 0.94 mmol) in DMF (10 mL) and water (2.5 mL) were added (2-fluorophenyl)boronic acid (197 mg, 1.41 mmol), and K₃PO₄ (598 mg, 2.82 mmol) at r.t. The resulting mixture was degassed by 3 cycles of vacuum/nitrogen. Sphos (154 mg, 0.38 mmol) and Sphos.Pd.G3 (146.5 mg, 0.19 mmol) were added to above mixture under a nitrogen atmosphere. The resulting mixture was stirred at 60° C. for 5 h under N₂. LCMS showed the reaction completed. The reaction mixture was taken up in EA (200 mL), and the organics were washed with water and saturated brine solution. The organic layers were then separated and dried over sodium sulfate and concentrated. The crude product was then purified by Prep-TLC to obtain 6-(2-fluorophenyl)pyrrolo[1,2-b]pyridazin-4-ol (200 mg, 93%) as a grey solid. MS: [M+H] Calc'd for C₁₃H₉FN₂O, 229. Found 229.

### Preparation 4.3B: [6-(2-fluorophenyl)-7aH-cyclopenta[b]pyridin-4-yl]trifluoromethanesulfonate

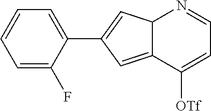 [SMILES: O=S(=O)(OC1=CC=NC2C=C(c3ccccc3F)C=C12)C(F)(F)F]

To a stirred solution of 6-(2-fluorophenyl)pyrrolo[1,2-b]pyridazin-4-ol (195 mg, 0.85 mmol) in DCM (10 mL) was added TEA (1.19 mL, 8.54 mmol). To this solution was added Tf₂O (0.29 mL, 1.71 mmol) dropwise at 0° C. The solution was warmed to r.t. for 3 h. LCMS showed depletion of starting material. The mixture was diluted with EA and washed with water. The organic layers were dried over sodium sulphate and concentrated under vacuum. The residue was purified by Prep-TLC (PE/EA=10/1) to obtain [6-(2-fluorophenyl)-7aH-cyclopenta[b]pyridin-4-yl] trifluoromethanesulfonate (210 mg, 68%) as a green solid. MS: [M+H] Calc'd for C₁₅H₉F₄NO₃S, 360. Found 360.

To a stirred solution of [6-(2-fluorophenyl)pyrrolo[1,2-b]pyridazin-4-yl]trifluoromethanesulfonate (170 mg, 0.47 mmol) in THF (8 mL) and water (2 mL) were added 2-cyclopentyl-4-(4,4,5,5-tetramethyl-1,3,2-dioxaborolan-2-yl)benzoic acid (224 mg, 0.71 mmol) and K₃PO₄ (300 mg, 1.42 mmol). The resulting mixture was degassed by 3 cycles of vacuum/nitrogen. Xphos.Pd.G3 (80 mg, 0.09 mmol) was then added to above mixture under a nitrogen atmosphere. The resulting mixture was stirred at r.t. for 7 h. TLC showed consumption of starting material and formation of new polar spot. The reaction mixture was concentrated, and then diluted with ethyl acetate and water. The pH of the solution was adjusted to 4-5 with a solution of HCl (2 mol/L), and then the resulting mixture was sequentially washed with water and saturated NaCl solution. The organic layer was dried over sodium sulphate and concentrated. The residue was purified by Prep-TLC to obtain crude product. Then the crude product was purified by Prep-HPLC (Column: XBridge Prep OBD C18 Column 30×150 mm 5 um; Mobile Phase A: Water (10 mmol/L NH₄HCO₃+0.1% NH₃·H₂O), Mobile Phase B: ACN; Flow rate: 60 mL/min; Gradient: 26% B to 45% B in 7 min; 254/210 nm; Rt: 5.83 min) to give 2-cyclopentyl-4-[6-(2-fluorophenyl)pyrrolo[1,2-b]pyridazin-4-yl]benzoic acid (62.8 mg, 32.5%) as a yellow solid. MS: [M+H] Calc'd for C₂₅H₂₁FN₂O₂, 401. Found 401. ¹H NMR (400 MHz, DMSO-d₆) δ 8.41 (d, J=2.0 Hz, 1H), 8.29 (d, J=4.7 Hz, 1H), 7.91 (t, J=7.4 Hz, 1H), 7.82 (d, J=7.8 Hz, 2H), 7.73-7.76 (m, 1H), 7.36-7.20 (m, 3H), 7.05 (s, 1H), 6.88 (d, J=4.7 Hz, 1H), 3.85-3.76 (m, 1H), 2.09 (s, 2H), 1.81 (s, 2H), 1.66 (s, 4H).

### II. CaMKK2 Assay

The ability of test compounds to bind and inhibit the activity of CaMKK2 were determined using a binding assay using LanthaScreent (Thermofisher) detection reagents in a 384-well plate format using the following assay buffer: 50 mM HEPES (pH7.5), 10 mM MgCl₂, 1 mM EGTA, and 0.01% Brij-35.

The assay reaction was initiated in the presence of 1 nM CaMKK2, 2 nM Eu-anti-GST antibody (Thermofisher), and 5 nM Kinase tracer 236 with Alexa Fluor647 (Thermofisher). A 10-point serial dilution of inhibitor compound in 0.5% DMSO was added to each well of the plate. The final assay volume in well was 15 uL. Assay mixture was incubated for 20 minutes at room temperature before reading on a BMG Pherastar plate reader with TR-FRET module (337 nm Excitation, 615 nm and 665 nm Emission). A ratio was calculated from the 2 readouts and fitted to a 4-parameter fit to determine the binding IC₅₀. See Table 6.

TABLE 6

| Chemical | | |
|---|---|---|
| Synthesis | | CaMKK2 |
| Example | Name | IC₅₀ |
| 1 | 2-Cyclopentyl-4-(3-phenylpyrrolo[2,3- | A |
| | b]pyrazin-5-yl)benzoic acid | |
| 2 | 2-Cyclopentyl-4-[3-(3- | A |
| | cyclopropylphenyl)pyrrolo[2,3-b]pyrazin-5- | |
| | yl]benzoic acid | |
| 3 | 4-[3-(3-Chlorophenyl)pyrrolo[2,3-b]pyrazin-5- | A |
| | yl]-2-cyclopentyl-benzoic acid | |
| 4 | 2-Cyclopentyl-4-(3-(m-tolyl)-5H-pyrrolo[2,3- | A |
| | b]pyrazin-5-yl)benzoic acid | |
| 5 | 2-(Ethylamino)-4-(3-phenylpyrrolo[2,3- | A |
| | b]pyrazin-5-yl)benzoic acid | |
| 6 | 2-((Cyclopropylmethyl)amino)-4-(3-phenyl- | A |
| | 5H-pyrrolo[2,3-b]pyrazin-5-yl)benzoic acid | |
| 7 | 2-(Cyclopentylamino)-4-(3-phenyl-5H- | A |
| | pyrrolo[2,3-b]pyrazin-5-yl)benzoic acid | |
| 8 | 2-((Cyclopropylmethyl)amino)-4-(3-(6-fluoro- | A |
| | 1-methyl-1H-indazol-5-yl)-5H-pyrrolo[2,3- | |
| | b]pyrazin-5-yl)benzoic acid | |
| 9 | 4-(3-(2,4-Difluorophenyl)-5H-pyrrolo[2,3- | A |
| | b]pyrazin-5-yl)-2-(ethylamino)benzoic acid | |
| 10 | 2-(Cyclopropylamino)-4-(3-(2,4- | A |
| | difluorophenyl)-5H-pyrrolo[2,3-b]pyrazin-5- | |
| | yl)benzoic acid | |
| 11 | 2-((Cyclopropylmethyl)amino)-4-(3-(4- | A |
| | fluorophenyl)-5H-pyrrolo[2,3-b]pyrazin-5- | |
| | yl)benzoic acid | |
| 12 | 2-(Cyclopropylmethylamino)-4-(3-(2-methyl- | A |
| | 2H-indazol-5-yl)-5H-pyrrolo[2,3-b]pyrazin-5- | |
| | yl)benzoic acid | |
| 13 | 2-(Ethylamino)-4-(3-(2-methyl-2H-indazol-5- | A |
| | yl)-5H-pyrrolo[2,3-b]pyrazin-5-yl)benzoic acid | |
| 14 | 2-(Ethylamino)-4-(3-(1-methyl-1H-indazol-5- | A |
| | yl)-5H-pyrrolo[2,3-b]pyrazin-5-yl)benzoic acid | |
| 15 | 2-(Cyclopropylamino)-4-(3-(4-fluorophenyl)- | A |
| | 5H-pyrrolo[2,3-b]pyrazin-5-yl)benzoic acid | |
| 16 | 2-(Ethylamino)-4-(3-(2-methyl-2H-indazol-6- | A |
| | yl)-5H-pyrrolo[2,3-b]pyrazin-5-yl)benzoic acid | |
| 17 | 2-((Cyclopropylmethyl)amino)-4-(3-(2-methyl- | A |
| | 2H-indazol-6-yl)-5H-pyrrolo[2,3-b]pyrazin-5- | |
| | yl)benzoic acid | |
| 18 | 2-Cyclopentyl-4-(3-cyclopropyl-5H- | A |
| | pyrrolo[2,3-b]pyrazin-5-yl)benzoic acid | |
| 19 | N-Cyano-2-cyclopentyl-4-(3-cyclopropyl-5H- | A |
| | pyrrolo[2,3-b]pyrazin-5-yl)benzamide | |
| 20 | 5-(3-cyclopentyl-4-(2H-tetrazol-5-yl)phenyl)- | A |
| | 3-cyclopropyl-5H-pyrrolo[2,3-b]pyrazine | |
| 21 | 2-Cyclopentyl-4-(3-cyclopropyl-5H- | B |
| | pyrrolo[2,3-b]pyrazin-5-yl)-N- | |
| | ((trifluoromethyl)sulfonyl)benzamide | |
| 22 | 2-Cyclopentyl-4-(3-phenyl-5H-pyrrolo[2,3- | C |
| | b]pyrazin-5-yl)benzonitrile | |
| 23 | 5-(3-Cyclopentyl-4-(2H-tetrazol-5-yl)phenyl)- | A |
| | 3-phenyl-5H-pyrrolo[2,3-b]pyrazine | |
| 24 | 2-(azetidin-3-yloxy)-4-(3-(3,5- | B |
| | dimethylphenyl)-5H-pyrrolo[2,3- | |
| | b]pyrazin-5-yl)benzoic acid | |
| 25 | 2-(azetidin-3-ylamino)-4-(3-(3,5- | A |
| | dimethylphenyl)-5H-pyrrolo[2,3- | |
| | b]pyrazin-5-yl)benzoic acid | |
| 26 | (R)and (S)-4-(3-(3,5-dimethylphenyl)-5H- | A |
| | pyrrolo[2,3-b]pyrazin-5-yl)- | |
| | 2-(pyrrolidin-3-yl) benzoic acid | |
| 27 | 2-(azetidin-3-ylmethyl)-4-(3-(3,5- | A |
| | dimethylphenyl)-5H-pyrrolo[2,3-b] | |
| | pyrazin-5-yl) benzoic acid | |
| 28 | (R) and (S)-4-(3-(3,5-dimethylphenyl)-5H- | A |
| | pyrrolo[2,3-b]pyrazin-5-yl)-2- | |
| | (piperidin-3-yl) benzoic acid | |
| 29 | 4-(3-(3,5-dimethylphenyl)-5H-pyrrolo[2,3- | A |
| | b]pyrazin-5-yl)-2-(piperidin-4-yl)benzoic | |
| | acid | |
| 30 | 4-(3-(3,5-dimethylphenyl)-5H-pyrrolo[2,3- | A |
| | b]pyrazin-5-yl)-2-(piperidin-4-yl)benzoic | |
| | acid | |
| 31 | (R)-4-(3-(3,5-dimethylphenyl)-5H- | A |
| | pyrrolo[2,3-b]pyrazin-5-yl)-2-(pyrrolidin- | |
| | 3-ylamino)benzoic acid | |
| 32 | (S)-4-(3-(3,5-dimethylphenyl)-5H- | A |
| | pyrrolo[2,3-b]pyrazin-5-yl)-2-(pyrrolidin- | |
| | 3-ylamino)benzoic acid | |
| 33 | (R)-4-(3-(3,5-dimethylphenyl)-5H- | A |
| | pyrrolo[2,3-b]pyrazin-5-yl)-2-(piperidin-3- | |
| | ylamino)benzoic acid | |
| 34 | 4-(3-(3,5-dimethylphenyl)-5H-pyrrolo[2,3- | A |
| | b]pyrazin-5-yl)-2-(piperidin-4- | |
| | ylamino)benzoic acid | |
| 35 | (R)-4-(3-(3,5-dimethylphenyl)-5H- | B |
| | pyrrolo[2,3-b]pyrazin-5-yl)-2-(pyrrolidin- | |
| | 3-yloxy)benzoic acid | |
| 2.1 | 2-cyclopentyl-4-(2- | A |
| | cyclopropylpyrazolo[1,5-a]pyrimidin-7- | |
| | yl)benzoic acid | |
| 2.2 | 2-cyclopentyl-4-(2-phenylpyrazolo[1,5- | A |
| | a]pyrimidin-7-yl)benzoic acid | |
| 2.3 | 2-cyclopentyl-4-(2-methoxypyrazolo[1,5- | A |
| | a]pyrimidin-7-yl)benzoic acid | |
| 2.4 | 2-cyclopentyl-4-(2-ethoxypyrazolo[1,5- | A |
| | a]pyrimidin-7-yl)benzoic acid | |
| 2.5 | 2-cyclopentyl-4-(2-(4- | A |
| | fluorophenyl)pyrazolo[1,5-a]pyrimidin-7- | |
| | yl)benzoic acid | |
| 2.6 | 2-cyclopentyl-4-(2-(2- | A |
| | fluorophenyl)pyrazolo[1,5-a]pyrimidin-7- | |
| | yl)benzoic acid | |
| 2.7 | 2-cyclopentyl-4-(2-(3- | A |
| | fluorophenyl)pyrazolo[1,5-a]pyrimidin-7- | |
| | yl)benzoic acid | |
| 2.8 | 2-cyclopentyl-4-(2-(4- | A |
| | methoxyphenyl)pyrazolo[1,5-a]pyrimidin- | |
| | 7-yl)benzoic acid | |
| 2.9 | 2-cyclopentyl-4-(2-(3- | A |
| | methoxyphenyl)pyrazolo[1,5-a]pyrimidin- | |
| | 7-yl)benzoic acid | |
| 2.10 | 2-cyclopentyl-4-(2-(3- | A |
| | ethoxyphenyl)pyrazolo[1,5-a]pyrimidin-7- | |
| | yl)benzoic acid | |
| 2.11 | 2-cyclopentyl-4-(2-(3- | A |
| | (cyclopropylmethoxy)phenyl)pyrazolo[1,5- | |
| | a]pyrimidin-7-yl)benzoic acid | |
| 2.12 | 2-cyclopentyl-4-(2-(1-methyl-1H-pyrazol- | A |
| | 4-yl)pyrazolo[1,5-a]pyrimidin-7-yl)benzoic | |
| | acid | |
| 2.13 | (S)-4-(2-(4-fluorophenyl)pyrazolo[1,5- | A |
| | a]pyrimidin-7-yl)-N-(2-hydroxy-1- | |
| | phenylethyl)-2-methoxybenzamide | |
| 3.1 | 2-Cyclopentyl-4-[2-(4- | A |
| | fluorophenyl)pyrazolo[4,3-b]pyridin-7- | |
| | yl]benzoic acid | |
| 3.2 | 2-Cyclopentyl-4-(2-phenyl-2H- | A |
| | pyrazolo[4,3-b]pyridin-7-yl)benzoic acid | |
| 3.3 | 2-Cyclopentyl-4-(2-(m-tolyl)-2H- | A |
| | pyrazolo[4,3-b]pyridin-7-yl)benzoic acid | |
| 3.4 | 2-Cyclopentyl-4-(2-(3-cyclopropylphenyl)- | A |
| | 2H-pyrazolo[4,3-b]pyridin-7-yl)benzoic | |
| | acid | |
| 3.5 | 2-Cyclopentyl-4-(6-fluoro-2-(4- | A |
| | fluorophenyl)-2H-pyrazolo[4,3-b]pyridin- | |
| | 7-yl)benzoic acid | |
| 3.6 | 4-(2-(3-Chlorophenyl)-2H-pyrazolo[4,3- | A |
| | b]pyridin-7-yl)-2-cyclopentylbenzoic acid | |
| 3.7 | 4-(2-(3-Chloro-5-methylphenyl)-6-fluoro- | A |
| | 2H-pyrazolo[4,3-b]pyridin-7-yl)-2- | |
| | cyclopentylbenzoic acid | |
| 3.8 | 2-Cyclopentyl-4-(6-fluoro-2-(3-methyl-5- | A |
| | (trifluoromethyl)phenyl)-2H-pyrazolo[4,3- | |
| | b]pyridin-7-yl)benzoic acid | |
| 3.9 | 2-Cyclopentyl-4-(6-fluoro-2-(3- | A |
| | (trifluoromethyl)phenyl)-2H-pyrazolo[4,3- | |
| | b]pyridin-7-yl)benzoic acid | |
| 3.10 | 4-(2-(3-Chloro-5-fluorophenyl)-6-fluoro- | A |
| | 2H-pyrazolo[4,3-b]pyridin-7-yl)-2- | |
| | cyclopentylbenzoic acid | |
| 3.11 | 2-Cyclopentyl-4-(6-fluoro-2-(3-fluoro-5- | A |
| | methylphenyl)-2H-pyrazolo[4,3-b]pyridin- | |
| | 7-yl)benzoic acid | |
| 3.12 | 2-Cyclopentyl-4-(2-(3,5-difluorophenyl)-6- | A |
| | fluoro-2H-pyrazolo[4,3-b]pyridin-7- | |
| | yl)benzoic acid | |
| 3.13 | 2-Cyclopentyl-4-(6-fluoro-2-(3-methoxy- | A |
| | 5-(trifluoromethyl)phenyl)-2H- | |
| | pyrazolo[4,3-b]pyridin-7-yl)benzoic acid | |
| 3.14 | 2-Cyclopentyl-4-(6-fluoro-2-(3-fluoro-5- | A |
| | (trifluoromethyl)phenyl)-2H-pyrazolo[4,3- | |
| | b]pyridin-7-yl)benzoic acid | |
| 3.15 | 4-(2-(3-Chloro-5-(trifluoromethyl)phenyl)- | A |
| | 6-fluoro-2H-pyrazolo[4,3-b]pyridin-7-yl)- | |
| | 2-cyclopentylbenzoic | |
| 3.16 | 2-Cyclopentyl-4-(2-(3,5-dichlorophenyl)- | A |
| | 6-fluoro-2H-pyrazolo[4,3-b]pyridin-7- | |
| | yl)benzoic acid | |
| 3.17 | 2-Cyclopentyl-4-(6-fluoro-2-(3-fluoro-5- | A |
| | methoxyphenyl)-2H-pyrazolo[4,3- | |
| | b]pyridin-7-yl)benzoic acid | |
| 3.18 | 2-Cyclopentyl-4-(6-fluoro-2-(3-methyl-5- | A |
| | (trifluoromethoxy)phenyl)-2H- | |
| | pyrazolo[4,3-b]pyridin-7-yl)benzoic acid | |
| 3.19 | (S)-4-(2-(4-Fluorophenyl)-2H- | A |
| | pyrazolo[4,3-b]pyridin-7-yl)-N-(2- | |
| | hydroxy-1-phenylethyl)-2- | |
| | methoxybenzamide | |
| 3.20 | N-Cyano-2-cyclopentyl-4-(2-(4- | A |
| | fluorophenyl)-2H-pyrazolo[4,3-b]pyridin- | |
| | 7-yl)benzamide | |
| 3.21 | 2-(Cyclopropylamino)-4-(6-fluoro-2-(4- | A |
| | fluorophenyl)-2H-pyrazolo[4,3-b]pyridin- | |
| | 7-yl)benzoic acid | |
| 4.1 | 2-Cyclopentyl-4-(6-phenylpyrrolo[1,2- | A |
| | b]pyridazin-4-yl)benzoic acid | |
| 4.2 | 2-Cyclopentyl-4-[6-(m-tolyl)pyrrolo[1,2- | A |
| | b]pyridazin-4-yl]benzoic acid | |
| 4.3 | 2-Cyclopentyl-4-[6-(2- | A |
| | fluorophenyl)pyrrolo[1,2-b]pyridazin-4- | |
| | yl]benzoic acid | |

Note:
Biochemical assay IC₅₀ data are designated within the following ranges:
A: ≤0.10 μM
B: >0.10 μM to ≤1.0 μM
C: >1.0 μM to ≤10 μM
D: >10 μM

### III. Cell-Based Phosphorylation Assay

Cellular CaMKK2 inhibition by compounds was measured by monitoring the phosphorylation of CaMKK2 substrate AMPK at Thr172 in A549 lung carcinoma cells (ATCC) using CISBIO Phospho-AMPK (Thr172) HTRF kit. A549 cells were plated in 96 well plates at 8000 cells/well in DMEM+10% FBS+1× pen/step+1×NEAA and incubated at 37° C. overnight in a CO₂ incubator. Cells were then treated with compounds for 4 hours. After cells were stimulated by 3 uM Calcium Ionophore (Sigma) at 37° C. for 30 minutes, cells were washed and then lysed with cell lysis buffer (CisBio) for 30 minutes. Lysates were then transferred to small volume white 384 assay plate and HTRF pAMPK(Thr172) detection reagents were added. Plates were incubated overnight and then read using HTRF mode by BMG PHERAstarFS.

### Cell Proliferation Assay

Proliferation was measured using CellTiter 96 Aqueous Non-Radioactive Cell Proliferation Assay Kit (Promega), which determines the number of viable cells in culture based on quantitation of dehydrogenase enzymes, as an indicator of metabolically active cells. A549 lung carcinoma cells were seeded at 2,000 cells/well in a 96-well tissue culture treated plate in DMEM+10% FBS+1× pen/step+1×NEAA and incubated at 37° C. overnight in a CO₂ incubator. Cells were treated with compounds for 48 hours. Then viability was measured using CellTiter 96 Aqueous Non-Radioactive Cell Proliferation Assay Kit (Promega). Detection was performed using Biotek Cytation 5 Cell Imaging Multi Mode plate reader. See Table 7.

TABLE 7

| Chemical | | | |
|---|---|---|---|
| Synthesis | | Cellular IC₅₀ | Cellular IC₅₀ |
| Example | Name | Phosphorylation | Proliferation |
| 1 | 2-Cyclopentyl-4-(3-phenylpyrrolo[2,3- | A | D |
| | b]pyrazin-5-yl)benzoic acid | | |
| 2 | 2-Cyclopentyl-4-[3-(3- | A | D |
| | cyclopropylphenyl)pyrrolo[2,3-b]pyrazin-5- | | |
| | yl]benzoic acid | | |
| 3 | 4-[3-(3-Chlorophenyl)pyrrolo[2,3-b]pyrazin-5- | B | D |
| | yl]-2-cyclopentyl-benzoic acid | | |
| 5 | 2-(Ethylamino)-4-(3-phenylpyrrolo[2,3- | B | D |
| | b]pyrazin-5-yl)benzoic acid | | |
| 6 | 2-((Cyclopropylmethyl)amino)-4-(3-phenyl- | B | D |
| | 5H-pyrrolo[2,3-b]pyrazin-5-yl)benzoic acid | | |
| 7 | 2-(Cyclopentylamino)-4-(3-phenyl-5H- | C | D |
| | pyrrolo[2,3-b]pyrazin-5-yl)benzoic acid | | |
| 9 | 4-(3-(2,4-Difluorophenyl)-5H-pyrrolo[2,3- | B | D |
| | b]pyrazin-5-yl)-2-(ethylamino)benzoic acid | | |
| 12 | 2-(Cyclopropylmethylamino)-4-(3-(2-methyl- | B | D |
| | 2H-indazol-5-yl)-5H-pyrrolo[2,3-b]pyrazin-5- | | |
| | yl)benzoic acid | | |
| 13 | 2-(Ethylamino)-4-(3-(2-methyl-2H-indazol-5- | B | D |
| | yl)-5H-pyrrolo[2,3-b]pyrazin-5-yl)benzoic acid | | |
| 18 | 2-Cyclopentyl-4-(3-cyclopropyl-5H- | B | D |
| | pyrrolo[2,3-b]pyrazin-5-yl)benzoic acid | | |
| 19 | N-Cyano-2-cyclopentyl-4-(3-cyclopropyl-5H- | C | D |
| | pyrrolo[2,3-b]pyrazin-5-yl)benzamide | | |
| 20 | 5-(3-cyclopentyl-4-(2H-tetrazol-5-yl)phenyl)- | D | D |
| | 3-cyclopropyl-5H-pyrrolo[2,3-b]pyrazine | | |
| 24 | 2-(azetidin-3-yloxy)-4-(3-(3,5- | A | D |
| | dimethylphenyl)-5H-pyrrolo[2,3- | | |
| | b]pyrazin-5-yl)benzoic acid | | |
| 2.2 | 2-cyclopentyl-4-(2-phenylpyrazolo[1,5- | B | D |
| | a]pyrimidin-7-yl)benzoic acid | | |
| 2.6 | 2-cyclopentyl-4-(2-(2- | B | D |
| | fluorophenyl)pyrazolo[1,5-a]pyrimidin-7- | | |
| | yl)benzoic acid | | |
| 2.10 | 2-cyclopentyl-4-(2-(3- | B | D |
| | ethoxyphenyl)pyrazolo[1,5-a]pyrimidin-7- | | |
| | yl)benzoic acid | | |
| 2.11 | 2-cyclopentyl-4-(2-(3- | B | D |
| | (cyclopropylmethoxy)phenyl)pyrazolo[1,5- | | |
| | a]pyrimidin-7-yl)benzoic acid | | |
| 3.1 | 2-Cyclopentyl-4-[2-(4- | B | D |
| | fluorophenyl)pyrazolo[4,3-b]pyridin-7- | | |
| | yl]benzoic acid | | |
| 3.2 | 2-Cyclopentyl-4-(2-phenyl-2H- | A | D |
| | pyrazolo[4,3-b]pyridin-7-yl)benzoic acid | | |
| 3.3 | 2-Cyclopentyl-4-(2-(m-tolyl)-2H- | B | D |
| | pyrazolo[4,3-b]pyridin-7-yl)benzoic acid | | |
| 3.4 | 2-Cyclopentyl-4-(2-(3-cyclopropylphenyl)- | A | D |
| | 2H-pyrazolo[4,3-b]pyridin-7-yl)benzoic | | |
| | acid | | |
| 3.5 | 2-Cyclopentyl-4-(6-fluoro-2-(4- | B | D |
| | fluorophenyl)-2H-pyrazolo[4,3-b]pyridin- | | |
| | 7-yl)benzoic acid | | |
| 3.6 | 4-(2-(3-Chlorophenyl)-2H-pyrazolo[4,3- | B | D |
| | b]pyridin-7-yl)-2-cyclopentylbenzoic acid | | |
| 3.7 | 4-(2-(3-Chloro-5-methylphenyl)-6-fluoro- | A | D |
| | 2H-pyrazolo[4,3-b]pyridin-7-yl)-2- | | |
| | cyclopentylbenzoic acid | | |
| 3.9 | 2-Cyclopentyl-4-(6-fluoro-2-(3- | B | D |
| | (trifluoromethyl)phenyl)-2H-pyrazolo[4,3- | | |
| | b]pyridin-7-yl)benzoic acid | | |
| 3.12 | 2-Cyclopentyl-4-(2-(3,5-difluorophenyl)-6- | B | D |
| | fluoro-2H-pyrazolo[4,3-b]pyridin-7- | | |
| | yl)benzoic acid | | |
| 3.16 | 2-Cyclopentyl-4-(2-(3,5-dichlorophenyl)- | A | D |
| | 6-fluoro-2H-pyrazolo[4,3-b]pyridin-7- | | |
| | yl)benzoic acid | | |
| 3.17 | 2-Cyclopentyl-4-(6-fluoro-2-(3-fluoro-5- | B | D |
| | methoxyphenyl)-2H-pyrazolo[4,3- | | |
| | b]pyridin-7-yl)benzoic acid | | |
| 3.18 | 2-Cyclopentyl-4-(6-fluoro-2-(3-methyl-5- | B | D |
| | (trifluoromethoxy)phenyl)-2H- | | |
| | pyrazolo[4,3-b]pyridin-7-yl)benzoic acid | | |
| 4.1 | 2-Cyclopentyl-4-(6-phenylpyrrolo[1,2- | B | D |
| | b]pyridazin-4-yl)benzoic acid | | |

Note:
Cellular assay IC₅₀ data are designated within the following ranges:
A: ≤0.10 μM
B: >0.10 μM to ≤1.0 μM
C: >1.0 μM to ≤10 μM
D: >10 μM

### IV. Evaluation of Quinazoline/Quinoline Based CaMKK2 Inhibitors

The properties of a series of quinazoline/quinoline based CaMKK2 inhibitors including their ability to bind and inhibit the activity of CaMKK2 are shown in Table 8. These test compounds exhibit potent and selective inhibition for CaMKK2 with strong pAMPK activity. These test compounds exhibit favorable absorption, distribution, metabolism and elimination (ADME) and physiochemical properties. The in vivo efficacy of these compounds is demonstrated in E0771 syngeneic model.

TABLE 8

| | Chemical Synthesis Example | | | | |
|---|---|---|---|---|---|
| | 5.1 | | 5.2 | 5.3 | 5.4 |
| Name | 2-cyclopentyl-4-(7- | | 2-cyclopentyl-4- | 2-cyclopentyl-4- | 4-(7-ethoxy-3- |
| | ethoxyquinazolin- | | (7-ethoxy-5- | (7-ethoxyquinolin- | fluoroquinolin- |
| | 4-yl)benzoic acid | | fluoroquinazolin- | 4-yl)benzoic acid | 4-yl)-2- |
| | | | 4-yl)benzoic | | isopropylbenzoic |
| | | | acid | | acid |
| CaMKK2 | | | | | |
| IC₅₀ (nM) | 6.6 | (n = 5) | 2.5 | 1.5 | 1.1 |
| CaMKK1 | 26 | (n = 5) | 12 | 1.7 | 1.7 |
| IC₅₀ (nM) | | | | | |
| A549 | 490 | (n = 4) | 170 | 60 | 79 |
| pAMPK | 74 | (n = 4) | TBD | 30 | TBD |

| | | | | |
|---|---|---|---|---|
| MOA EC₅₀ | | | | |
| (nM) | | | | |
| A427 | | | | |
| pAMPK | | | | |
| MOA EC₅₀ | | | | |
| (nM) | | | | |
| Kinase | 0 of 72 @ 0.3 mM | 0 of 72 @ | 6 of 72 @ | 6 of 72 @ |
| Selectivity | 3 of 259 @ 3 mM | 0.3 mM | 3 mM | 3 mM |
| KSP (72)/ | (Abl 648 nM) | | | |
| FP (259) | | | | |
| CYP450 | >30 mM except | | | |
| | 2C9 (4 mM) | | | |
| PPB (m, h) | 98.5, 99% | | >99% | TBD |
| hERG | >30 mM | | | |
| Microsomes | Stability₆₀: 96, | | CYP/UGT₆₀: 66, | |
| (m, h) | 100% | | 67% | |
| | UGT: 79, 89% | | | |
| PK | AUC/dose = 10,500 | | AUC/dose = 1,650 | TBD |
| | % F = 80% | | % F = 58% | |
| CEREP | no inhibition >50% | | | |
| | @ 10 uM | | | |

### Enumerated Embodiments

1. A compound of Formula (I), or a pharmaceutically acceptable salt thereof,

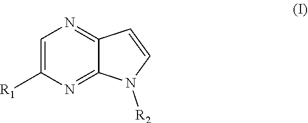

wherein,
   R₁ is alkyl, cycloalkyl, optionally substituted aryl, optionally substituted heteroaryl, or amine; and
   R₂ is optionally substituted aryl.
2. The compound of claim 1, wherein R₁ is C3 cycloalkyl.
3. The compound of claim 1, wherein R₁ is aryl optionally substituted by halogen, alkyl, or cycloalkyl.
4. The compound of claim 1, wherein R₁ is aryl optionally substituted by methyl.
5. The compound of claim 1, wherein R₁ is aryl optionally substituted by C3 cycloalkyl.
6. The compound of claim 1, wherein R₁ is heteroaryl optionally substituted by halogen or alkyl.
7. The compound of claim 1, wherein R₁ is heteroaryl optionally substituted by methyl.
8. The compound of claim 1, wherein R₁ is bicyclic heteroaryl.
9. The compound of claim 1, wherein R₂ is aryl optionally substituted by at least one substituent chosen from cycloalkyl, carbonyl, amine, —CN, heterocyclyl, heterocyclyloxy, heterocyclylalkyl, or heteroaryl.
10. The compound of claim 9, wherein the cycloalkyl is C5-cycloalkyl.
11. The compound of claim 9, wherein the carbonyl is chosen from —C(═O)OH, —C(═O)NH—CN, or —C(═O)NH—SO₂—CF₃.
12. The compound of claim 9, wherein the amine is chosen from —NH-alkyl, —NH-cycloalkyl, —NH-cycloalkylalkyl, or —NH-heterocyclyl.
13. The compound of claim 9, wherein the heterocyclyl is 5-6 membered heterocyclyl containing N.
14. The compound of claim 9, wherein the heteroaryl is tetrazole.
15. A compound of Formula (IIa), or a pharmaceutically acceptable salt thereof,

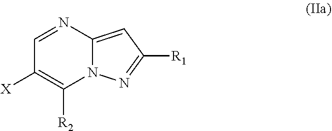

wherein,
   R₁ is alkoxy, cycloalkyl, optionally substituted aryl, optionally substituted heteroaryl, or amine;
   R₂ is optionally substituted aryl; and
   X is hydrogen or halogen.
16. The compound of claim 15, wherein R₁ is alkoxy chosen from methoxy or ethoxy.
17. The compound of claim 15, wherein R₁ is C3 cycloalkyl.
18. The compound of claim 15, wherein R₁ is aryl optionally substituted by halogen and alkoxy.
19. The compound of claim 15, wherein R₁ is aryl optionally substituted by methoxy.
20. The compound of claim 15, wherein R₁ is aryl optionally substituted by —O-cycloalkylalkyl.
21. The compound of claim 15, wherein R₁ is heteroaryl optionally substituted by methyl.
22. The compound of claim 15, wherein R₁ is pyrazole substituted by methyl.
23. The compound of claim 15, wherein R₂ is aryl optionally substituted by at least one substituent chosen from carbonyl, alkoxy, or cycloalkyl.
24. The compound of claim 23, wherein the carbonyl is —C(═O)OH or —C(═O)-amine.
25. The compound of claim 23, wherein the alkoxyl is methoxy.
26. The compound of claim 23, wherein the cycloalkyl is C5 cycloalkyl.
27. A compound of Formula (IIb), or a pharmaceutically acceptable salt thereof,

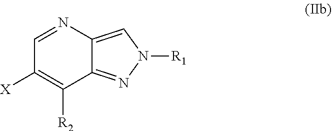

wherein,
   R₁ is optionally substituted aryl;
   R₂ is optionally substituted aryl; and
   X is hydrogen or halogen.
28. The compound of claim 27, wherein R₁ is aryl optionally substituted by at least one substituent chosen from halogen, alkyl, CF₃, alkoxy, —O—CF₃, or cycloalkyl.
29. The compound of claim 27, wherein R₁ is aryl optionally substituted by methyl.
30. The compound of claim 27, wherein R₁ is aryl optionally substituted by methoxy.
31. The compound of claim 27, wherein R₁ is aryl optionally substituted by C3-cycloalkyl.
32. The compound of claim 27, wherein R₂ is aryl optionally substituted by at least one substituent chosen from carbonyl, alkoxy, amine, or cycloalkyl.
33. The compound of claim 32, wherein the carbonyl is chosen from —C(═O)OH or —C(═O)-amine.
34. The compound of claim 32, wherein the alkoxy is methoxy.
35. The compound of claim 32, wherein the amine is —NH-cycloalkyl.
36. The compound of claim 32, wherein the cycloalkyl is C5 cycloalkyl.
37. A compound of Formula (IIc), or a pharmaceutically acceptable salt thereof,

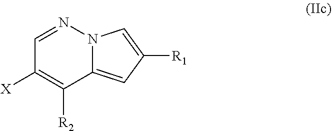

wherein,
   R₁ is optionally substituted aryl;
   R₂ is optionally substituted aryl; and
   X is hydrogen or halogen.
38. The compound of claim 37, wherein R₁ is aryl optionally substituted by at least one substituent chosen from halogen or alkyl.
39. The compound of claim 37, wherein R₁ is aryl optionally substituted by methyl.
40. The compound of claim 37, wherein R₂ is aryl optionally substituted by at least one substituent chosen from carbonyl or cycloalkyl.
41. The compound of claim 40, wherein the carbonyl is —C(═O)OH.
42. The compound of claim 40, wherein the cycloalkyl is C5 cycloalkyl.
43. A compound of Formula (III), or a pharmaceutically acceptable salt thereof,

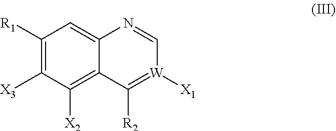

wherein,
   W is carbon or nitrogen;
   R₁ is alkoxy, cyclylalkoxy, heterocyclyl, or heteroaryl;
   R₂ is optionally substituted aryl; and
   X₁, X₂, and X₃ is independently hydrogen or halogen.
44. The compound of claim 43, wherein R₁ is ethoxy.
45. The compound of claim 43, wherein R₁ is C3 cyclylalkoxy.
46. The compound of claim 43, wherein R₁ is C5 heterocyclyl containing at least one nitrogen.
47. The compound of claim 43, wherein R₁ is C5 heteroaryl containing at least one nitrogen.
48. The compound of claim 43, wherein R₂ is aryl optionally substituted by at least one substituent chosen from halogen, carbonyl, cycloalkyl, or heterocyclyl.
49. The compound of claim 48, wherein the carbonyl is —C(═O)OH.
50. The compound of claim 48, wherein the cycloalkyl is C₃-C₅ cycloalkyl.
51. The compound of claim 48, wherein the heterocyclyl is C₃-C₆ heterocyclyl containing at least one nitrogen.
52. A pharmaceutical composition comprising a compound of Formula (I) of claim 1, or a pharmaceutically acceptable salt thereof, and a pharmaceutically acceptable excipient.
53. A pharmaceutical composition comprising a compound of Formula (IIa) of claim 15, or a pharmaceutically acceptable salt thereof, and a pharmaceutically acceptable excipient.
54. A pharmaceutical composition comprising a compound of Formula (IIb) of claim 27, or a pharmaceutically acceptable salt thereof, and a pharmaceutically acceptable excipient.
55. A pharmaceutical composition comprising a compound of Formula (IIc) of claim 37, or a pharmaceutically acceptable salt thereof, and a pharmaceutically acceptable excipient.
56. A pharmaceutical composition comprising a compound of Formula (III) of claim 43, or a pharmaceutically acceptable salt thereof, and a pharmaceutically acceptable excipient.
57. A method of regulating gene transcription in a cell comprising inhibiting Calcium/calmodulin-dependent protein kinase kinase 2 activity by exposing the Calcium/calmodulin-dependent protein kinase kinase 2 enzyme to a compound of Formula (I) of claim 1.
58. A method of regulating gene transcription in a cell comprising inhibiting Calcium/calmodulin-dependent protein kinase kinase 2 activity by exposing the Calcium/calmodulin-dependent protein kinase kinase 2 enzyme to a compound of Formula (IIa) of claim 15.
59. A method of regulating gene transcription in a cell comprising inhibiting Calcium/calmodulin-dependent protein kinase kinase 2 activity by exposing the Calcium/calmodulin-dependent protein kinase kinase 2 enzyme to a compound of Formula (IIb) of claim 27.
60. A method of regulating gene transcription in a cell comprising inhibiting Calcium/calmodulin-dependent protein kinase kinase 2 activity by exposing the Calcium/calmodulin-dependent protein kinase kinase 2 enzyme to a compound of Formula (IIc) of claim 37.
61. A method of regulating gene transcription in a cell comprising inhibiting Calcium/calmodulin-dependent protein kinase kinase 2 activity by exposing the Calcium/calmodulin-dependent protein kinase kinase 2 enzyme to a compound of Formula (III) of claim 43.
62. A method of treating cancer in a patient in need thereof, comprising administering to the patient a therapeutically effective amount of a compound of Formula (I) of claim 1, or a pharmaceutically acceptable salt thereof.
63. A method of treating cancer in a patient in need thereof, comprising administering to the patient a therapeutically effective amount of a compound of Formula (IIa) of claim 15, or a pharmaceutically acceptable salt thereof.
64. A method of treating cancer in a patient in need thereof, comprising administering to the patient a therapeutically effective amount of a compound of Formula (IIb) of claim 27, or a pharmaceutically acceptable salt thereof.
65. A method of treating cancer in a patient in need thereof, comprising administering to the patient a therapeutically effective amount of a compound of Formula (IIc) of claim 37, or a pharmaceutically acceptable salt thereof.
66. A method of treating cancer in a patient in need thereof, comprising administering to the patient a therapeutically effective amount of a compound of Formula (III) of claim 43, or a pharmaceutically acceptable salt thereof.

1. A compound of Formula (I), or a pharmaceutically acceptable salt thereof, 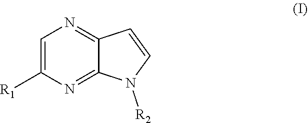
wherein,
R₁ is alkyl, cycloalkyl, optionally substituted aryl, optionally substituted heteroaryl, or amine; and
R₂ is optionally substituted aryl.

2. The compound of claim 1, wherein R₂ is aryl optionally substituted by at least one substituent chosen from cycloalkyl, carbonyl, amine, —CN, heterocyclyl, heterocyclyloxy, heterocyclylalkyl, or heteroaryl.

3. A compound of Formula (IIa), or a pharmaceutically acceptable salt thereof, 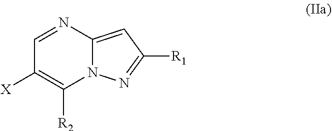
wherein,
R₁ is alkoxy, cycloalkyl, optionally substituted aryl, optionally substituted heteroaryl, or amine;
R₂ is optionally substituted aryl; and
X is hydrogen or halogen.

4. The compound of claim 15, wherein R₂ is aryl optionally substituted by at least one substituent chosen from carbonyl, alkoxy, or cycloalkyl.

5. A compound of Formula (IIb), or a pharmaceutically acceptable salt thereof, 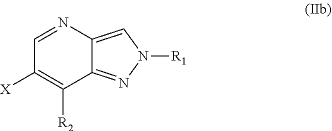
wherein,
R₁ is optionally substituted aryl;
R₂ is optionally substituted aryl; and
X is hydrogen or halogen.

6. The compound of claim 5, wherein R₁ is aryl optionally substituted by at least one substituent chosen from halogen, alkyl, CF₃, alkoxy, —O—CF₃, or cycloalkyl.

7. The compound of claim 5, wherein R₂ is aryl optionally substituted by at least one substituent chosen from carbonyl, alkoxy, amine, or cycloalkyl.

8. A compound of Formula (IIc), or a pharmaceutically acceptable salt thereof, 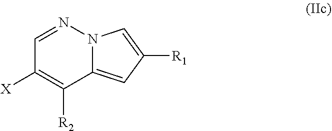
wherein,
R₁ is optionally substituted aryl;
R₂ is optionally substituted aryl; and
X is hydrogen or halogen.

9. The compound of claim 8, wherein R₁ is aryl optionally substituted by at least one substituent chosen from halogen or alkyl.

10. The compound of claim 8, wherein R₂ is aryl optionally substituted by at least one substituent chosen from carbonyl or cycloalkyl.

11. A compound of Formula (III), or a pharmaceutically acceptable salt thereof, 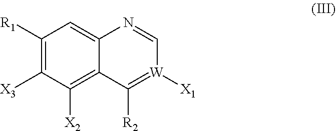
wherein,
W is carbon or nitrogen;
R₁ is alkoxy, cyclylalkoxy, heterocyclyl, or heteroaryl;
R₂ is optionally substituted aryl; and
X₁, X₂, and X₃ is independently hydrogen or halogen.

12. The compound of claim 11, wherein R₂ is aryl optionally substituted by at least one substituent chosen from halogen, carbonyl, cycloalkyl, or heterocyclyl.

13. A pharmaceutical composition comprising a compound according to claim 1, or a pharmaceutically acceptable salt thereof, and a pharmaceutically acceptable excipient.

14. A method of regulating gene transcription in a cell comprising inhibiting Calcium/calmodulin-dependent protein kinase kinase 2 activity by exposing the Calcium/calmodulin-dependent protein kinase kinase 2 enzyme to a compound according to claim 1.

15. A method of treating cancer in a patient in need thereof, comprising administering to the patient a therapeutically effective amount of a compound according to claim 1, or a pharmaceutically acceptable salt thereof.
